(19) United States
(12) Patent Application Publication
(10) Pub. No.: US 2025/0361214 A1
(43) Pub. Date: Nov. 27, 2025

(54) TRIAZINE LIPIDS, LIPID SYNTHESIS, AND METHODS FOR INHIBITING CANONICAL NFKB TRANSCRIPTIONAL ACTIVITY

(71) Applicant: University of Kentucky Research Foundation, Lexington, KY (US)

(72) Inventors: Vincent J. Venditto, Lexington, KY (US); David Nardo Padron, Philadelphia, PA (US); Abdullah Al Masud, Lexington, KY (US); Julian Agustin Mory, Lexington, KY (US)

(21) Appl. No.: 18/992,921

(22) PCT Filed: Jul. 13, 2023
(86) PCT No.: PCT/US2023/027667

§ 371 Date: Jan. 9, 2025

Related U.S. Application Data
(60) Provisional application No. 63/388,768, filed on Jul. 13, 2022.

(51) Int. Cl. C07D 251/54; A61K 31/53; A61K 31/5377; C12N 15/117

(52) U.S. Cl. CPC C07D 251/54; A61K 31/53; A61K 31/5377; C12N 15/117; C12N 2310/17

(57) ABSTRACT

Triazine lipids and solid-phase synthesis methods for synthesizing triazine lipids are provided. Certain triazine lipids disclosed can inhibit canonical NF_{K}B transcriptional activity and thus may be utilized in non-viral transfection vectors for administration with an immunostimulatory antigen to reduce reactogenic response invoked by the immunostimulatory antigen. Methods for inhibiting canonical NF_{K}B transcriptional activity during an immune response are also provided.

### CROSS-REFERENCE TO RELATED APPLICATION

The present application claims priority to U.S. Patent Application Ser. No. 63/388,768 filed on Jul. 13, 2022, the entire disclosure of which is incorporated herein by reference.

### TECHNICAL FIELD

The presently disclosed subject matter relates to lipids which can be administered with an immunostimulatory antigen. More particularly, the presently disclosed subject matter relates to triazine lipids which can be utilized in non-viral vectors for administration with immunostimulatory antigens, and which inhibit canonical NF_{K}B transcriptional activity during an immune response. Methods for synthesizing triazine lipids are also describe herein.

### BACKGROUND

Liposomes provide an optimal vehicle for pharmaceutical delivery due to their versatility as amphipathic vectors, which allows for delivery of hydrophobic and hydrophilic agents.^{1,2} By altering the lipid composition in these nanoparticles, a multitude of properties can be honed to optimize their functionality. In the last few decades, liposome research has fueled the development of synthetic lipids to improve therapeutic delivery, particularly nucleic acids.³ However, the complexity and cost of novel lipids limits liposome research.^{1,4-6} To overcome this, various research groups have developed synthetic, cationic lipid libraries with the goal of improving siRNA and mRNA delivery using cost effective and high-throughput schemes, taking advantage of specific chemical structures that allow for rapid headgroup diversification.⁵⁻⁷

In addition to their utility as gene delivery vectors, liposomes have been investigated extensively for vaccine development using nucleic acids or proteins^{8,9} both as adjuvants,¹⁰ and as anchors of antigens on liposomal surfaces.¹¹ Incorporating adjuvants and antigens in the same formulation has also improved antigen exposure to immune cells and enhanced the efficacy of liposomal vaccines.^{12,13}

The ability of lipid based nanoparticles to form transfection vehicles is dependent on the ionic interaction between cationic lipids and nucleic acids, which allows the nanoparticle to deliver the nucleic acid payload into cells.³⁵ This field has been largely expanded by the work of various researchers who have elucidated the structure activity relationship of cationic lipids and have implemented design elements to optimize gene delivery.³⁶⁻³⁸ Due to the protein levels and transgene immunogenicity achieved with mRNA, versus plasmid DNA, this type of nucleic acid has become prevalent for liposomal gene delivery, particularly in the context of vaccines.⁴² However, the immunogenic potential of mRNA can deter its use in other forms of gene therapy, such as gene replacement, where the development of anti-transgene antibodies can lead to clearance and failure of therapies. Plasmid delivery might therefore have an advantage in this context, since it can lead to reduced immunogenicity^{43,44} and it does not result in immune system activation, similar to modified mRNA based nanoparticles.^{45,46}

The public awareness of lipid-based vaccines has been fully realized due to the COVID-19 pandemic and the development of mRNA-based vaccines to induce protective immunity. Lipid carriers have been investigated extensively as immunomodulators since haptenated lipids were first formulated in 1974.^{129, 130} The modular format of lipid delivery systems provides a platform for inclusion of hydrophobic or hydrophilic adjuvants in a nanoparticle to increase antigen retention at sites of injection, improve immune recognition, and immune cell uptake.¹⁰⁸ The versatility of this platform has led to licensed SARS-COV-2 vaccines as well as approved subunit vaccines for influenza, malaria, shingles, and human papilloma virus (HPV), with several additional formulations in clinical development.

The adjuvants currently approved by the United States Federal Drug Administration (FDA) for use alone or in lipid-based vaccines include alum, monophosphoryl lipid A (MPLA), cytosine phosphoguanine (CpG), saponins, squalene, and combinations of each.¹³¹⁻¹³³ Adjuvant induced immune response can be characterized both by the robustness of immune responses and the TH1/TH2 balance as measured by antibody profiles (e.g. IgG2a/IgG1).^{134, 135} TH1 responses are primarily classified as cell-mediated immunity, and opsonizing antibodies (e.g. IgG2a) are a marker of this response. Conversely, TH2 responses are typically classified as humoral responses, and antibodies induced for protection against extracellular pathogens (e.g. IgG1) mark this response. Overactive TH1 responses can cause tissue damage and uncontrolled TH2 responses can cause allergic responses.¹³⁶ Therefore, a balanced, antigen-specific TH1/TH2 response represents the ideal vaccine induced immune profile. Saponin (e.g. QS-21) is the only approved adjuvant that elicits a balanced TH1/TH2 response.¹³⁷ Alum is skewed heavily toward a TH2 response, while MPLA, CpG, and squalene are skewed heavily toward a TH1 response.¹³¹ Therefore, there is a critical need to expand the portfolio of adjuvanted delivery systems that promote balanced immunity for vaccine development, and evaluation of their mechanism of action is necessary for preclinical development in relevant models.

The adjuvants included in lipid-based vaccines target specific innate immune pathways associated with robust immune responses. The underlying premise builds from the concept that nanoparticles containing adjuvants are recognized and taken up by antigen presenting cells, stimulated through pattern recognition receptors (PRRs) that leads to activation of B cells and T cells.^{138, 139} Under this premise, increased cytokine expression and antigen-specific antibody responses are used as markers of successful immune activation. However, the resultant immune response associated with these formulations is marked by both protective immunity and a reactogenic response associated with “flu-like” symptoms.¹⁴⁰ Such undesirable responses result in subjects who prefer not to be vaccinated. 141 Vaccine-induced reactogenicity is most commonly observed side-effect of vaccination with increased levels of pyrogenic cytokines (e.g. IL-6, TNFα) that promote pain, swelling and redness at the sight of injection as well as headache, fever, myalgia, and fatigue.¹⁴⁰ Therefore, strategies that promote protective immunity using small molecule immune modulators, while limiting reactogenicity, are desirable.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In one aspect, the presently disclosed subject matter includes a triazine lipid. In some embodiments, the triazine lipid is of the formula

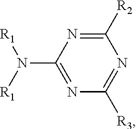

wherein: R₁ is alkyl; R₂ is

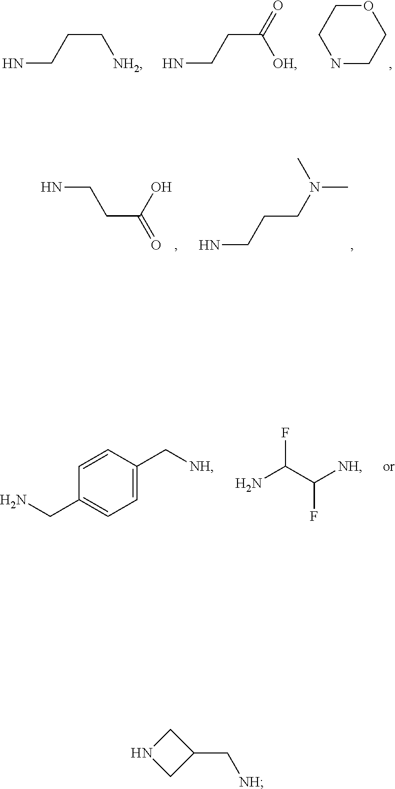

and R₃ is NH₂,

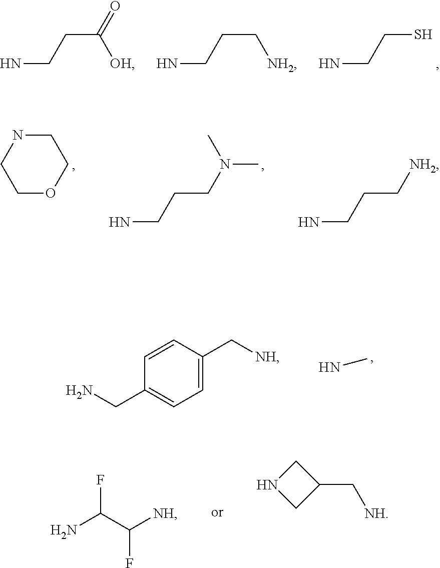

In some embodiments, R₁ comprises at least 12 alkyl carbons. In some embodiments, R₁ comprises 18 alkyl carbons. In some embodiments, R₁ comprises 12 alkyl carbons. In some embodiments, R₂ and R₃ each comprise

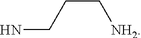

In one embodiment, the triazine lipid is

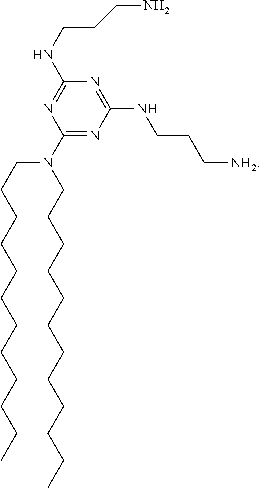 [SMILES: CCCCCCCCCCCCN(CCCCCCCCCCCC)c1nc(NCCCN)nc(NCCCN)n1]

In another embodiment, the triazine lipid is

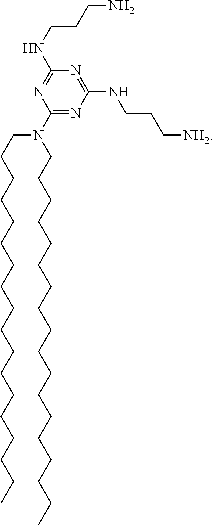 [SMILES: CCCCCCCCCCCCCCCCCCN(CCCCCCCCCCCCCCCCCC)c1nc(NCCCN)nc(NCCCN)n1]

In another aspect, the presently disclosed subject matter also includes a non-viral triazine lipid-based vector including a plurality of triazine lipids consistent with one or more of the above-identified lipids.

In another aspect, the presently disclosed subject matter also includes a solid-phase synthesis method for synthesizing triazine lipids. In the method, a resin is reacted with a first amine headgroup to generate an amine terminated resin. In some embodiments, the resin is an amine-reactive resin. A dichlorotriazine is then formulated substituting the amine terminated resin with a cyanuric chloride via nucleophilic aromatic substitution. The dichlorotriazine is then reacted with a lipid tail to form a monochlorotriazine, which is subsequently reacted with a second amine group to form the triazine lipid. After formation, the triazine lipid is cleaved from the resin.

In another aspect, the presently disclosed subject matter also includes a method for inhibiting canonical NF_{K}B transcriptional activity during an immune response to an immunostimulatory antigen within a subject. In some embodiments, the method includes administering a non-viral triazine lipid-based vector including a plurality of triazine lipids to a subject concurrently with an immunostimulatory antigen. In some embodiments, the immunostimulatory antigen is an immunogenic peptide. In some embodiments, the immunostimulatory antigen is an immunostimulatory nucleic acid. In some embodiments, each lipid of the plurality of triazine lipids is cationic. In some embodiments, the triazine lipid includes a lipid tail group, a triazine linker, and a cationic headgroup. In some embodiments, the lipid tail comprises a saturated or unsaturated dialkylamine. In some embodiments, each triazine lipid of the non-viral vector is of the formula

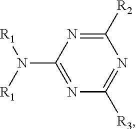

wherein: R₁ is alkyl; R₂ is

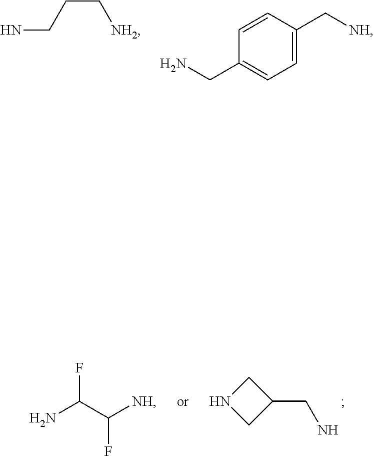

and R₃ is NH₂,

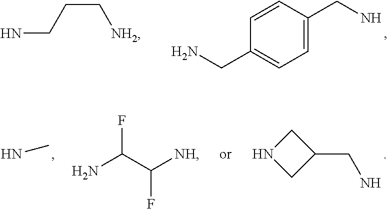

In some embodiments, R₁ comprises 18 or fewer alkyl carbons. In some embodiments, R₁ comprises at least 12 alkyl carbons. In some embodiments, wherein R₁ comprises 18 alkyl carbons. In some embodiments, R₁ comprises 12 alkyl carbons. In some embodiments, the non-viral triazine lipid-based vector is a liposome. In some embodiments the non-viral triazine lipid-based vector administered to the subject may include one or more additional lipids selected from the group consisting of dioleoylphosphatidylethanolamine (DOPE), distearolyphosphatidycholine (DSPC), and 1, 2-distearoyl-sn-glycero-3-phsphoethanolamine-polyethylene glycol (DSPE-PEG). In some embodiments, the non-viral triazine lipid-based vector administered in the method may stimulate non-canonical NF_{K}B transcriptional activity. In one such embodiment, each triazine lipid of the plurality of triazine lipids is

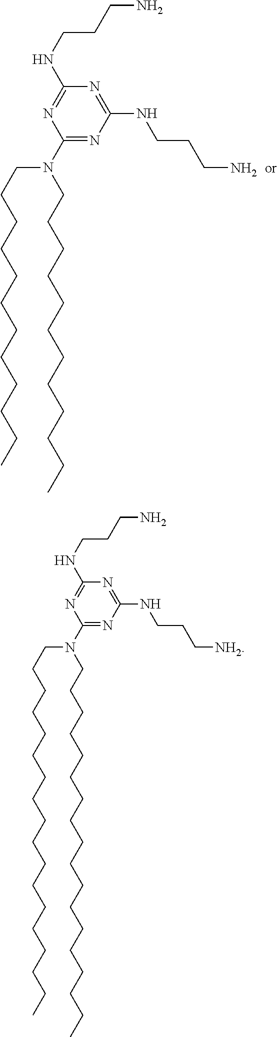 [SMILES: CCCCCCCCCCCCN(CCCCCCCCCCCC)c1nc(NCCCN)nc(NCCCN)n1 ; CCCCCCCCCCCCCCCCCCN(CCCCCCCCCCCCCCCCCC)c1nc(NCCCN)nc(NCCCN)n1]

In some embodiments, the immunostimulatory antigen is an immunogenic polypeptide and the non-viral triazine-based vector invokes an anti-polypeptide response which is greater than an anti-polypeptide response invoked by dioleoyl-3-trimethylammonium propone (DOTAP) or 1,2-Dimyristoyl-sn-glycero-3-phosphocholine (DMPC) when administered concurrently with the immunogenic polypeptide. In some embodiments, the non-viral triazine lipid-based vector and the immunostimulatory antigen may be administered together as an immunogenic composition including the non-viral triazine lipid-based vector and the immunostimulatory antigen.

In yet another aspect, the presently disclosed subject matter also includes a method for inhibiting canonical transcriptional activity during an immune response within one or more cells. In some embodiments, the method includes contacting the one or more cells with one or more triazine lipids.

Further features and advantages of the presently disclosed subject matter will become evident to those of ordinary skill in the art after a study of the description, figures, and non-limiting examples in this document.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1 shows synthetic schemes for producing triazine (TZ) lipids.

FIG. 2A is a graph showing the transition temperature of TZ lipids determined by DSC.

FIG. 2B is a graph showing in vitro toxicity of triazine lipids. Toxicity of TZ lipids on bone marrow-derived macrophages as compared to commercially available cationic (DOTMA) and zwitterionic (DMPC) lipids using the lactate dehydrogenase assay. Liposomes were made by thin film hydration followed by sonication and used immediately to treat cells for 24 hours, prior to testing LDH release in cell media. Representative data from one of three independent experiments is shown; bars indicate mean values for three technical replicates of duplicate experiments SEM.

FIG. 3 shows the efficacy of TZ lipids in gene transfection. (A) Gel shift assay of plasmid DNA complexed with TZ lipids. (B) pKa assessment of cationic lipids measured by TNS fluorescence at pH range 2.5 to 10. Plots represent the sigmoidal, best fit analysis of one of three independent experiments. (C-E) Transfection of HeLa cells with luciferase reporter gene using Lipofectamine 3000 or TZ lipids at an N:P ratio of 10, 5 and 2.5 (left to right). Bars represent the mean values from one of three representative experiments, except for the LDH assay which was performed twice. (C) Luciferase expression in transfected HeLa cells. (D) LDH release from HeLa cells transfected with luciferase plasmid 4 hours after transfection. (E) Viability of cells treated with plasmid and lipids 24 hours after transfection. (F-H) Transfection of HEK293-T cells with hAAT using Lipofectamine 3000 or TZ lipids at N:P ratios of 6, 3 and 1.5 (left to right). Bars represent the mean values from one of three representative experiments, except for the viability assay which was performed twice. (F) hAAT expression 72 hours after transfection based on ELISA and normalized to total cell protein. (G) LDH release from cells transfected with hAAT plasmid 24 hours after transfection. (H) Viability of cells treated with plasmid and lipids 48 hours after transfection. In both experiments, each treatment was compared to the Lipofectamine control using the Kruskal-Wallis nonparametric test. Bars indicate mean values for triplicates SEM and p=<0.05.

FIG. 4 shows TZ lipids as peptide anchors in liposomal vaccines. (A) Liposomal vaccines can include various components, including natural phospholipids and adjuvants, to optimize responses to an immunogen. (B) Lipid linkers anchoring apolipoprotein A-I peptide to the liposomal vaccine, cholesterol hemisuccinate and intermediate D (C18 TZ). (C) Reciprocal endpoint titers 7 days after the second of two immunizations compared with no peptide immunization. Symbols correspond to individual mice and line represents mean SEM and p=<0.05.

FIG. 5 shows that TZ3 does not induce significant in vivo toxicity at 20 mM. Seven-week-old C57BL/6J mice were administered 100 μL of 20 mM cationic lipid (TZ3, TZ9 or DOTAP (Do)) nanoparticles intraperitoneally in HEPES buffered saline. (A) Serum alanine aminotransferase (ALT), (B) Interleukin 6 (IL-6), and (C) serum creatinine (SCr) levels were measured 48 hours after treatment. Fold-change from baseline measurements drawn one week prior were compared with those of untreated animals (NT). Lines represent mean, and dots represent individual animals. Equal numbers of each sex were included; however, the TZ9 group represents only the 7 surviving animals. Significance was compared using one way ANOVA and Dunnett's (A) or Kruskal Wallis (B and C) tests; only significant comparisons are shown.

FIG. 6 shows that PEG550, DOPE, and TZ3 improve transfection efficiency with LNPs, but LPs exhibit improved transfection efficiency and reduced toxicity in vivo. (A-B) HEK293T cells were transfected with 200 ng GFP plasmid per well using LNPs and analyzed three days later for GFP expression by flow cytometry. (A) LNPs formulated with 50% TZ3, 10% DSPC, 39% cholesterol and 1% DSPE-PEG (550-2000), or 40% cholesterol and no PEG. (B) LNPs formulated with 50% DOTAP (Do) or TZ3, 10% DSPE or DOPE, 39% cholesterol and 1% DSPE-PEG550. Pooled data from three independent experiments is shown; n=3 transfected wells per group per experiment. (C-F) Male and female BALB/c mice were administered 1×109 genome copies of AAV8-GFP or 10 μg of GFP plasmid in either LNPs made with 50% TZ3, 10% DOPE, 39% cholesterol and 1% DSPE-PEG550 or LPs made with 50% TZ3 and 50% DOPE. One week post administration, hepatocytes were evaluated for: (C) percent GFP positive cells, or (D) mean fluorescence intensity (MFI). (E) Percent weight change and (F) serum ALT were also evaluated at the same time point. Bars indicate mean transfection efficiency+/−SD; dots represent individual transfection wells in (A) or mice in (C-F). Data were compared with one-way ANOVA and Dunnett's test in (A, C-F) or Sidak's text in (B); comparisons shown in (A) are to No PEG and in (C-F) to untreated mice (NT).

FIG. 7 shows that TZ3 LP transfection is more efficient in vivo than TZ3 LNPs or formulations made with DOTAP. BALB/c mice were administered 10 μg of hAAT DNA with LNPs made with 50% TZ3 or DOTAP (Do), 10% DOPE, 39% cholesterol and 1% DSPE-PEG550 or LPs made with 50% TZ3 and 50% DOPE. Seventy-two hours later, protein expression in the serum was assessed via ELISA. Lines represent mean hAAT concentration; dots represent individual animals. Data was compared with Kruskal-Wallis test.

FIG. 8 shows that transgene expression using TZ3 as a delivery vector elicits minimal antibody responses, while administration of hAAT protein with TZ3 results in significant immunogenicity. BALB/c mice were administered 10 μg of hAAT DNA with LNPs made with 50% TZ3 or DOTAP (Do), 10% DOPE, 39% cholesterol and 1% DSPE-PEG550 or LPs made with 50% TZ3 and 50% DOPE; or 25 μg of hAAT protein in saline or 1 mM lipid solution. (A) Fourteen days after administration, anti-hAAT IgG reciprocal endpoint titers (RET) were assessed via serum ELISA. Significance determined by Kruskal-Wallis test; comparisons made to untreated animals (NT). Ratios of IgG2a/IgG1 (B), IgG2b/IgG1 (C), and IgG3/IgG1 (D) were assessed at the same timepoint for treatment groups that had significantly higher RET than untreated. Bars indicate mean, while dots indicate individual animals. Significance as compared to protein delivered in saline was determined by one-way ANOVA in (B-D).

FIG. 9 shows that PEGylation decreases LNP uptake by antigen presenting cells. (A) Bone marrow-derived dendritic cells (DC) or J774 macrophages were incubated for 18 hours with LNPs made with 5% DiD and DSPE-PEG2000, or PEG-free liposomes. The percentage of cells positive for DiD fluorescence by flow cytometry is shown; data represent pooled results from three independent experiments, N=3 wells/treatment. (B) Bone marrow-derived dendritic cells (DC) or J774 macrophages were transfected with 200 ng of GFP DNA delivered with LPs made of 50% TZ3 and 50% DOPE; LNPs made with 50% TZ3, 10% DOPE, 40% cholesterol; or LNPs made with 50% TZ3, 10% DOPE, 39% cholesterol and 1% DSPE-PEG550. Seventy-two hours after transfection, the cells were analyzed by flow cytometry for GFP expression; data represents pooled results from three independent experiments, N=3 wells/treatment. Bars indicate mean+/−SD. Only statistically significant comparisons are shown. Significance determined by one-sample T-test in (A) or one-way ANOVA in (B). ND=not determined.

FIG. 10 shows intermediate compounds A-G for the synthesis of triazine lipids.

FIG. 11 shows ¹H Nuclear Magnetic Resonance (NMR) for intermediate compound A.

FIG. 12 shows ¹³C NMR for intermediate compound A.

FIG. 13 shows ¹H NMR for intermediate compound B.

FIG. 14 shows ¹³C NMR for intermediate compound B.

FIG. 15 shows ¹H NMR for 2-[(Triphenylmethyl)thio]ethanamine.

FIG. 16 shows ¹³C NMR for 2-[(Triphenylmethyl)thio]ethanamine.

FIG. 17 shows ¹H NMR for intermediate compound C.

FIG. 18 shows ¹³C NMR for intermediate compound C.

FIG. 19 shows ¹H NMR for intermediate compound D.

FIG. 20 shows ¹³C NMR for intermediate compound D.

FIG. 21 shows the ¹H NMR for intermediate compound E.

FIG. 22 shows ¹³C NMR for intermediate compound E.

FIG. 23 shows ¹H NMR for intermediate compound F.

FIG. 24 shows the ¹³C NMR for intermediate compound F.

FIG. 25 shows ¹H NMR for intermediate compound G.

FIG. 26 shows ¹³C NMR for intermediate compound G.

FIG. 27 shows ¹H NMR for lipid 1 (TZ1).

FIG. 28 shows ¹³C NMR for TZ1.

FIG. 29 shows ¹H NMR for lipid 2 (TZ2).

FIG. 30 shows ¹³C NMR for TZ2.

FIG. 31 shows ¹H NMR for lipid 3 (TZ3).

FIG. 32 shows ¹³C NMR for TZ3.

FIG. 33 shows ¹H NMR for lipid 4 (TZ4).

FIG. 34 shows ¹³C NMR for TZ4.

FIG. 35 shows ¹H NMR for lipid 5 (TZ5).

FIG. 36 shows ¹³C NMR for TZ5.

FIG. 37 shows ¹H NMR for lipid 6 (TZ6).

FIG. 38 shows ¹³C NMR for TZ6.

FIG. 39 shows ¹H NMR for lipid 7 (TZ7).

FIG. 40 shows ¹³C NMR for TZ7.

FIG. 41 shows ¹H NMR for lipid 8 (TZ8).

FIG. 42 shows ¹³C NMR for TZ8.

FIG. 43 shows ¹H NMR for lipid 9 (TZ9).

FIG. 44 shows the ¹³C NMR for TZ9.

FIG. 45 shows ¹H NMR for lipid 10 (TZ10).

FIG. 46 shows ¹³C NMR for TZ10.

FIG. 47 shows ¹H NMR for lipid 11 (TZ11).

FIG. 48 shows ¹³C NMR for TZ11.

FIG. 49 shows ¹H NMR for lipid 12 (TZ12).

FIG. 50 shows ¹³C NMR for TZ12.

FIG. 51 shows HPLC traces of lipids TZ1-TZ4 and chloroform (used as solvent), detected at 205 and 254 (254 shown). The mobile phase was a gradient of water and acetonitrile with 0.1% trifluoroacetic acid, as indicated, and constant 5% methanol with 01% trifluoroacetic acid. *Shortened due to speed of compound elution.

FIG. 52 shows HPLC traces of lipids TZ5-8 and chloroform (used as solvent), detected at 205 and 254 (254 shown). The mobile phase was a gradient of water and acetonitrile with 0.1% trifluoroacetic acid, as indicated, and constant 5% methanol with 01% trifluoroacetic acid. The four compounds corresponding to TZ5-8 needed a mixture of isopropanol and chloroform for proper dissolution of HPLC.

FIG. 53 shows HPLC traces of lipids TZ9-TZ12 and chloroform (used as solvent), detected at 205 and 254 (254 shown). The mobile phase was a gradient of water and acetonitrile with 0.1% trifluoroacetic acid, as indicated, and constant 5% methanol with 01% trifluoroacetic acid. *Shortened due to speed of compound elution.

FIG. 54 shows HPLC traces of free apolipoprotein A-I and apolipoprotein A-I lipopeptide.

FIG. 55 shows in vivo toxicity of TZ3 and TZ9 at 10 mM. Seven-week-old C57BL/6J mice were administered 100 μL of 10 mM cationic lipid (TZ3, T9, or DOTAP (Do)) intraperitoneally. (A) Serum alanine aminotransferase (ALT), (B) interleukin-6 (IL-6), and (C) serum creatinine (SCr) levels were measured 48 hours after treatment. Fold-change from baseline measurements drawn one week prior were compared with those of untreated animals (NT). Lines represent mean, and dots represent individual animals. Equal numbers of each sex were included; however, the TZ9 group represents only the 6 surviving animals. Significance was compared using one way ANOVA and Kruskal Wallis tests; only significant comparisons are shown.

FIG. 56 shows extent of mice transfection with hAAT plasmid administered in lipid nanoparticles including TZ3 or DOTAP (Do). Eight-week-old male and female BALB/c mice were administered 500 ng hAAT plasmid in lipid nanoparticles (LNPs) made with 50% cationic lipid (TZ3 or DOTAP (Do)), 10% DPSC, 39% cholesterol and 1% DSPE-PEG2000. Blood was drawn 72 hours after injection and hAAT protein levels were detected by ELISA. Dotted line indicates limit of quantification.

FIG. 57 shows (A) Four weeks after LP transfection, protein expression in the serum was assessed via ELISA. Only values above the limit of quantification are shown. (B) hAAT protein concentrations at 72 hours after direct administration of 25 μg hAAT protein in either saline or with 1 mM lipid nanoparticle solution (indicated). (C) Fold-change in serum ALT from baseline measurements at 72 hours after either transfection or protein delivery. Lines and bars represent mean; dots represent individual animals. Data are compared with one-way ANOVA and Kruskal-Wallis test; significance is as compared to protein in saline only, only significant comparisons are shown.

FIG. 58 shows schemes for flow cytometry analysis. (A) Scheme for GFP quantification in HEK293T cells stained with Zombie NearIR Dye after transfection with GFP plasmid with LNPs. (B) Scheme for GFP quantification in mouse splenocytes stained with anti-CD45-APC and anti-CD 146-PE/Cy7 after transfection with GFP plasmid in nanoparticles or AAV8. (C) DiD quantification in APCs stained with Zombie Green Dye after treatment with DiD liposomes. (D) GFP quantification in APCs stained with Zombie NearIR Dye after treatment with GFP plasmid in nanoparticles.

FIG. 59 is a diagram showing the canonical and non-canonical pathways for NF_{K}B stimulation. NF_{K}B stimulation is mediated through two distinct pathways (canonical and non-canonical), which are both regulated by CIAP1/2 in the basal state. Inhibition of IAP inhibits the canonical NF_{K}B pathway (reactogenic) and enhances the non-canonical NF_{K}B pathway (immunity) to improve vaccine induced protection.

FIG. 60 shows cationic triazine-based lipid (TZ3) induces an increase greater than 3 orders of magnitude in antibody titter toward a model antigen in the absence of additional adjuvants with a balanced TH1/TH2 response. Titers are shown for sham immunized mice, protein administered in saline, protein administered with DOTAP (DO), or protein administered with cationic triazine lipid (TZ3).

FIG. 61 is a schematic showing thermally controlled aromatic substitution of cyanuric chloride.

FIG. 62 shows (A) structures of triazine lipids used in toxicity experiments, (B) quantified by lactate dehydrogenase activity as a marker of early apoptosis. Bone marrow derived macrophages were incubated with lipids for 24 hours at decreasing concentrations (250, 125, 62.5, 31.5 μM). Media and triton are used as negative and positive controls, respectively. DO=dioctadecenyl-3-trimethylammonium propane; DMPC=dimyristoylphosphatidylcholine.

FIG. 63 shows the inhibition of canonical NF_{K}B activity in human THP-1-Blue cells stimulated with TNFα (shown) or LPS (not shown) using cationic triazine-based lipids containing primary amine headgroups (TZ3, TZ4). IC50 values: TZ3, 4 μM; DOTAP, 28 μM; TZ4, 29 μM. As shown, NF_{K}B inhibition is quantified after 24 h lipid treatment.

FIG. 64 is a diagram showing a solid-phase synthesis method for synthesizing a triazine lipid library.

FIG. 65 shows J774 murine macrophages treated with cationic triazine lipid (TZ3) for 16 hours exhibit does-dependent increase in mean fluorescence intensity (MFI) of CD80 relative to untreated cells.

FIG. 66 shows TZ3 inhibition of canonical NF_{K}B activity is dose dependent, but not affected by duration of exposure. As shown, NF_{K}B inhibition is comparable at 1-9 h.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently disclosed subject matter.

Unless defined otherwise, all technical and scientific terms used herein have the same meaning as commonly understood by one of ordinary skill in the art to which the disclosure belongs. Any methods and materials similar to or equivalent to those described herein can be used in the practice or testing of the present disclosure, including the methods and materials described below.

All patents, patent applications, published applications and publications, GenBank sequences, databases, websites, and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

Following long-standing patent law convention, the terms “a,” “an,” and “the” refer to “one or more” when used in this application, including the claims. Thus, for example, reference to “a cell” includes a plurality of cells, and so forth.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

When open-ended terms such as “including” or “including, but not limited to” are used, there may be other non-enumerated members of a list that would be suitable for the making, using or sale of any embodiment thereof.

As used herein, the term “immunostimulatory antigen” refers to a protein, peptide, or other molecule or macromolecule, in whole or in part, capable of eliciting an immune response. As such, the term “immunostimulatory antigen” can refer to, in some embodiments, an immunogenic polypeptide, and, in other embodiments, an immunostimulatory nucleic acid.

As used herein, the term “immunogenic polypeptide” refers to a polypeptide which, when introduced to a target cell, invokes a protective immune response, such as an inflammatory response or induction of cytokines. As used herein, the term “polypeptide” can refer to, in some embodiments, a polypeptide and, in other embodiments, a protein. As such, an “immunogenic polypeptide” can be, in some embodiments, a polypeptide which invokes an immune response, and, in other embodiments, a protein which invokes an immune response.

As used herein, the term “immunostimulatory nucleic acid” refers to a molecule of nucleotides which encodes for an immunogenic polypeptide or which otherwise invokes or enhances an immune response. In some embodiments, the immunostimulatory nucleic acid may be a deoxyribonucleic acid (DNA) molecule, while, in other embodiments, the immunostimulatory nucleic acid may be a ribonucleic acid (RNA) molecule (e.g., mRNA or siRNA).

As used herein “effective amount” in the context of inhibiting canonical Nuclear Factor Kappa B (NF_{K}B) transcriptional activity during an immune response with a non-viral triazine lipid-based vector refers to an amount of the non-viral triazine lipid-based vector, which, when administered to the subject, inhibits a reactogenic response within the subject induced by an immunostimulatory antigen, such as an immunogenic polypeptide or an immunostimulatory nucleic acid. In some embodiments of the disclosed methods, an effective amount of the non-viral triazine lipid-based vector may be administered concurrently with the immunostimulatory antigen, e.g., as part of a vaccine or other immunogenic composition.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A “patient” refers to a subject afflicted with a disease or disorder. The term “patient” includes human and veterinary subjects.

As used herein, the term “derivative” refers to a compound having a structure derived from the structure of a parent compound (e.g., a compound disclosed herein) and whose structure is sufficiently similar to those disclosed herein and based upon that similarity, and would be expected by one skilled in the art to exhibit the same or similar activities and utilities as the claimed compounds, or to induce, as a precursor, the same or similar activities and utilities as the claimed compounds.

As used herein, the term “pharmaceutically-acceptable carrier” refers to a solid or liquid filler, diluent, and/or encapsulating substance that may be safely administered to a subject to facilitate delivery of a composition.

Administration of the non-viral triazine lipid-based vectors disclosed herein can occur intravenously, intramuscularly, intraperitoneally, orally, or topically.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration, percentage, or the like is meant to encompass variations of in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, nomenclature for compounds, including organic compounds, can be given using common names, IUPAC, IUBMB, or CAS recommendations for nomenclature. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used herein, the term “optional” or “optionally” means that the subsequently described event or circumstance can or cannot occur, and that the description includes instances where said event or circumstance occurs and instances where it does not.

All combinations of method or process steps as used herein can be performed in any order, unless otherwise specified or clearly implied to the contrary by the context in which the referenced combination is made.

The presently disclosed subject matter includes triazine lipids which may be utilized as non-viral transfection vectors for administration with an immunostimulatory antigen. In some embodiments, a triazine lipid is of the formula

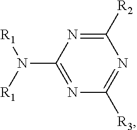

wherein: R₁ is alkyl; R₂ is

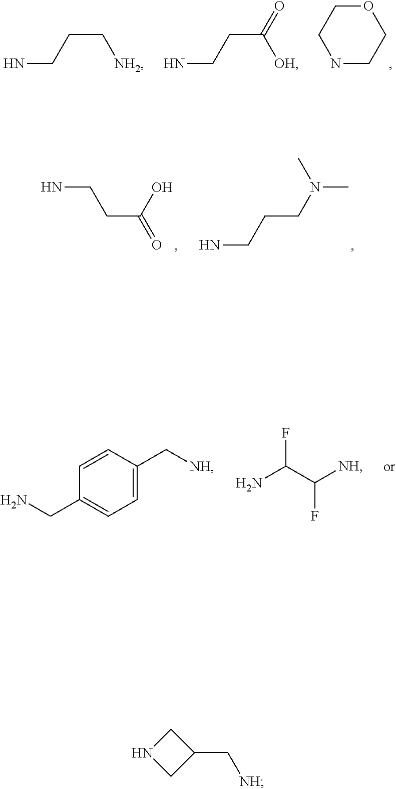

and R₃ is NH₂,

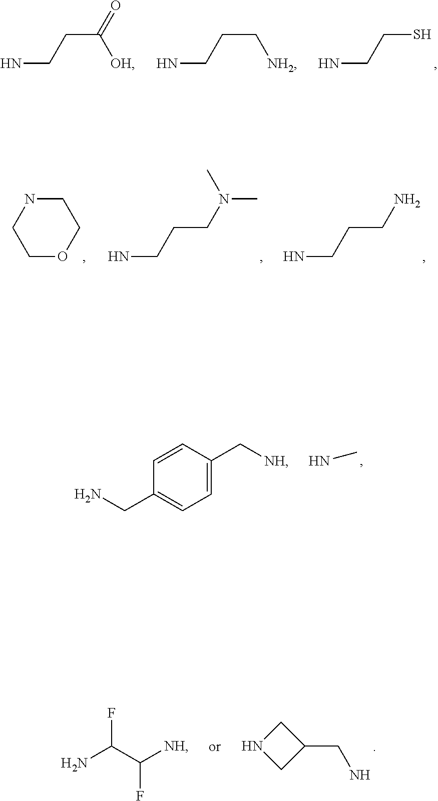

In some embodiments, R₁ comprises 18 or fewer alkyl carbons. In some embodiments, R₁ comprises at least 12 alkyl carbons. In some embodiments, R₁ comprises 18 alkyl carbons. In some embodiments, R₁ comprises 12 alkyl carbons. In some embodiments, R₂ and R₃ each comprise

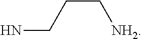

In some embodiments, the triazine lipid is

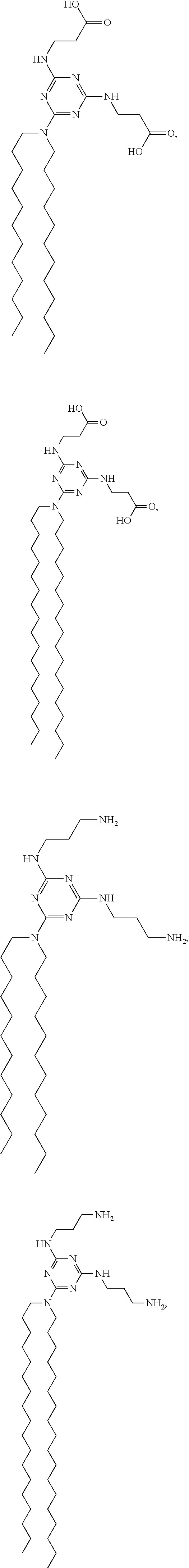 [SMILES: CCCCCCCCCCCCN(CCCCCCCCCCCC)c1nc(NCCC(=O)O)nc(NCCC(=O)O)n1 ; CCCCCCCCCCCCCCCCCCN(CCCCCCCCCCCCCCCCCC)c1nc(NCCC(=O)O)nc(NCCC(=O)O)n1 ; CCCCCCCCCCCCN(CCCCCCCCCCCC)c1nc(NCCCN)nc(NCCCN)n1 ; CCCCCCCCCCCCCCCCCCN(CCCCCCCCCCCCCCCCCC)c1nc(NCCCN)nc(NCCCN)n1]

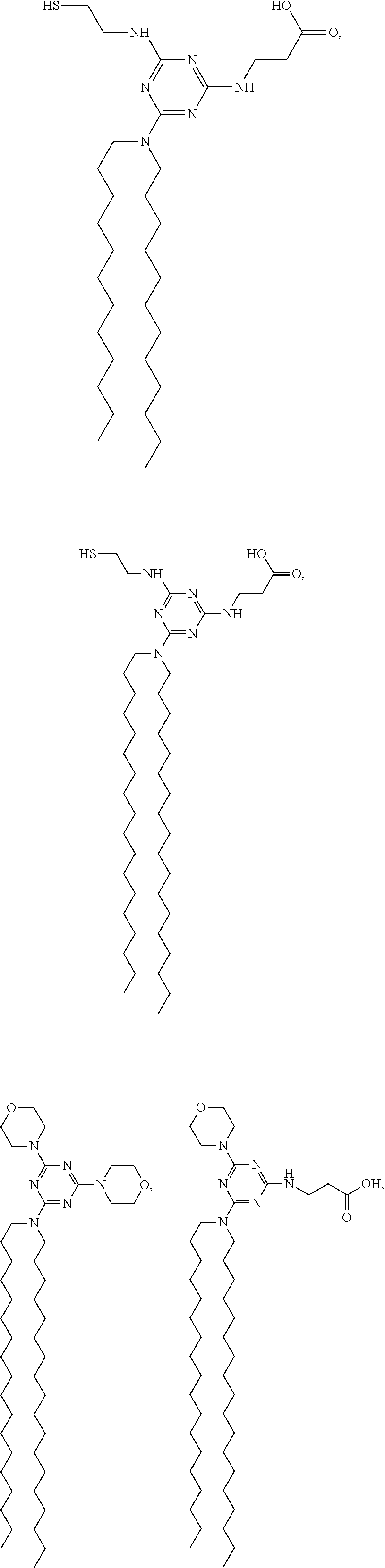 [SMILES: CCCCCCCCCCCCN(CCCCCCCCCCCC)c1nc(NCCS)nc(NCCC(=O)O)n1 ; CCCCCCCCCCCCCCCCCCN(CCCCCCCCCCCCCCCCCC)c1nc(NCCS)nc(NCCC(=O)O)n1 ; CCCCCCCCCCCCCCCCCCN(CCCCCCCCCCCCCCCCCC)c1nc(N2CCOCC2)nc(N2CCOCC2)n1 ; CCCCCCCCCCCCCCCCCCN(CCCCCCCCCCCCCCCCCC)c1nc(NCCC(=O)O)nc(N2CCOCC2)n1]

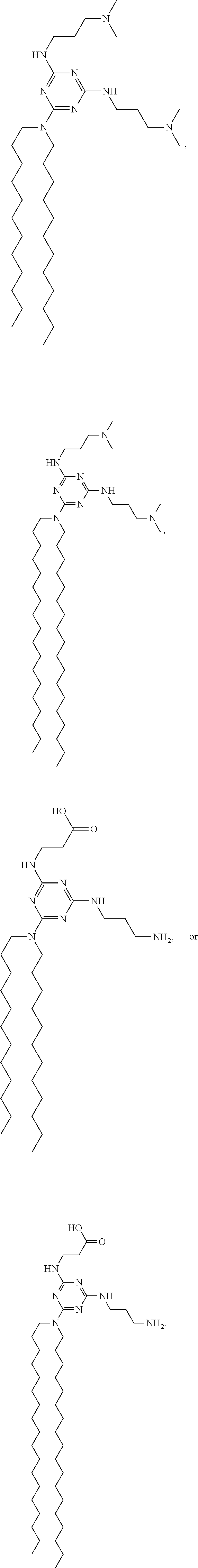 [SMILES: CCCCCCCCCCCCN(CCCCCCCCCCCC)c1nc(NCCCN(C)C)nc(NCCCN(C)C)n1 ; CCCCCCCCCCCCCCCCCCN(CCCCCCCCCCCCCCCCCC)c1nc(NCCCN(C)C)nc(NCCCN(C)C)n1 ; CCCCCCCCCCCCN(CCCCCCCCCCCC)c1nc(NCCCN)nc(NCCC(=O)O)n1 ; CCCCCCCCCCCCCCCCCCN(CCCCCCCCCCCCCCCCCC)c1nc(NCCCN)nc(NCCC(=O)O)n1]

In some embodiments, the triazine lipid is

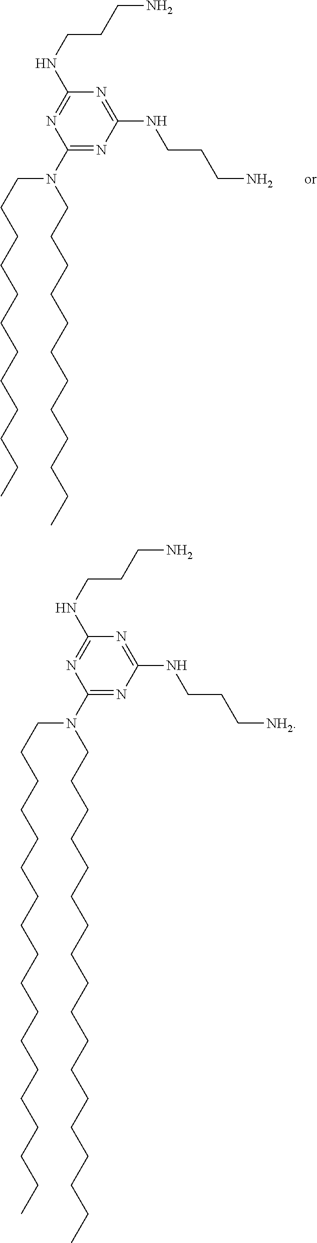 [SMILES: CCCCCCCCCCCCN(CCCCCCCCCCCC)c1nc(NCCCN)nc(NCCCN)n1 ; CCCCCCCCCCCCCCCCCCN(CCCCCCCCCCCCCCCCCC)c1nc(NCCCN)nc(NCCCN)n1]

The presently disclosed subject matter also includes non-viral triazine lipid-based vectors including a plurality of triazine lipids, where each lipid is of a formula consistent with that specified above. Accordingly, in some embodiments, each lipid of the plurality of triazine lipids of a vector may be of the formula

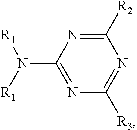

wherein: R₁ is alkyl; R₂ is

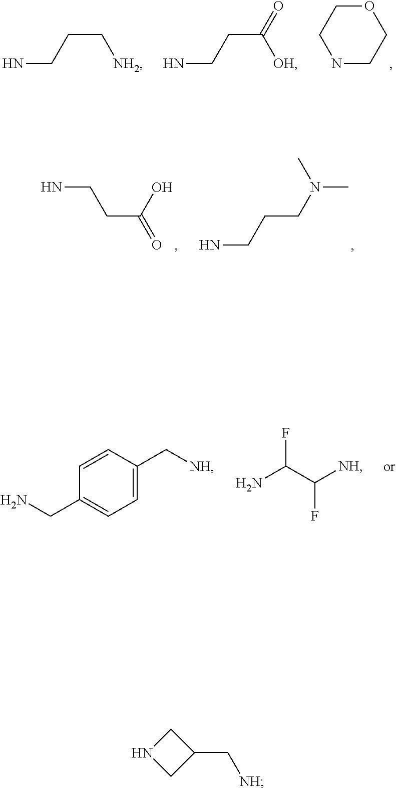

and R₃ is NH₂,

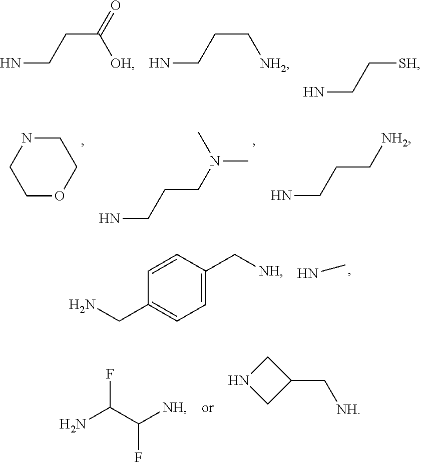

In some embodiments, R₁ of each triazine lipid of the vector comprises 18 or fewer alkyl carbons. In some embodiments, R₁ of each triazine lipid of the vector comprises at least 12 alkyl carbons. In some embodiments, R₁ of each triazine lipid of the vector comprises 18 alkyl carbons. In some embodiments, R₁ of each triazine lipid of the vector comprises 12 alkyl carbons. In some embodiments, R₂ and R₃ of each triazine lipid of the vector each comprise

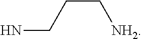

In some embodiments, each triazine lipid of the vector is

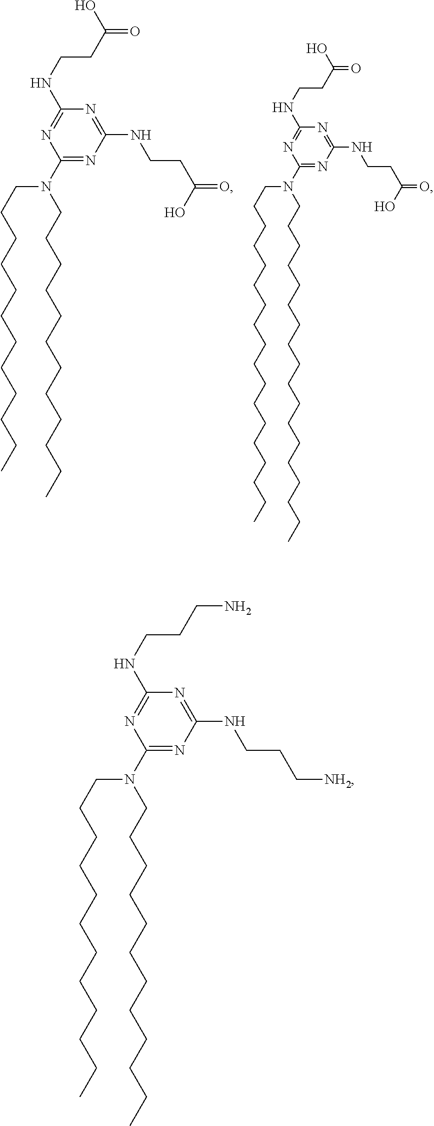

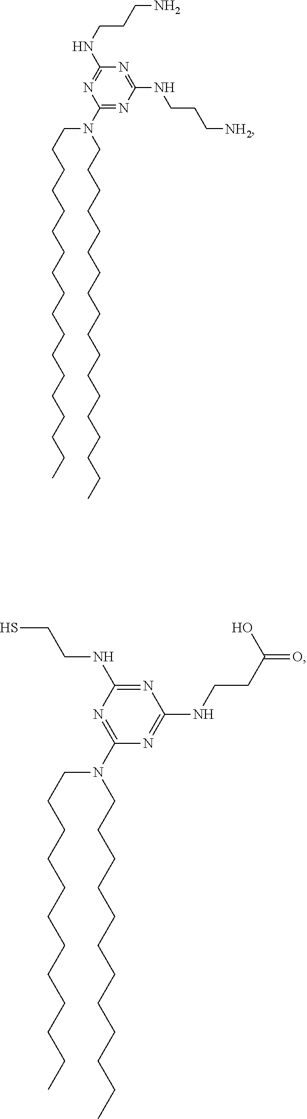 [SMILES: CCCCCCCCCCCCCCCCCCN(CCCCCCCCCCCCCCCCCC)c1nc(NCCCN)nc(NCCCN)n1 ; CCCCCCCCCCCCN(CCCCCCCCCCCC)c1nc(NCCS)nc(NCCC(=O)O)n1]

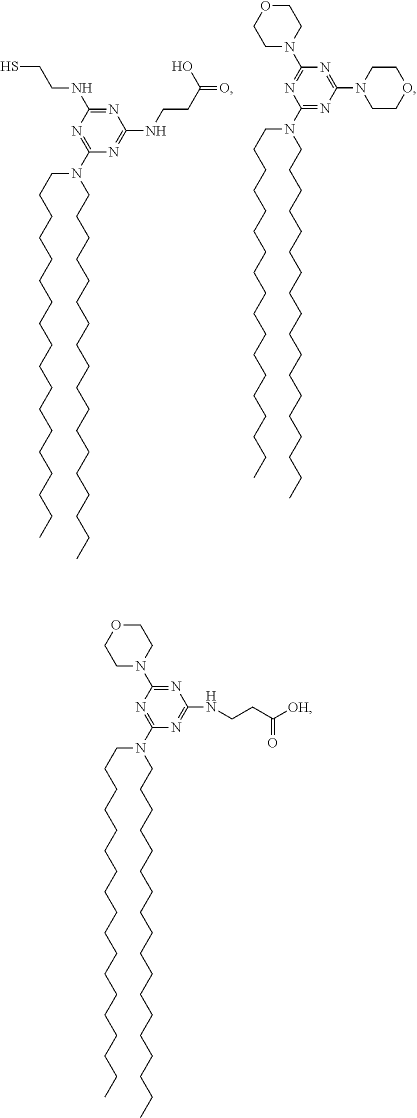 [SMILES: CCCCCCCCCCCCCCCCCCN(CCCCCCCCCCCCCCCCCC)c1nc(NCCS)nc(NCCC(=O)O)n1 ; CCCCCCCCCCCCCCCCCCN(CCCCCCCCCCCCCCCCCC)c1nc(N2CCOCC2)nc(N2CCOCC2)n1 ; CCCCCCCCCCCCCCCCCCN(CCCCCCCCCCCCCCCCCC)c1nc(NCCC(=O)O)nc(N2CCOCC2)n1]

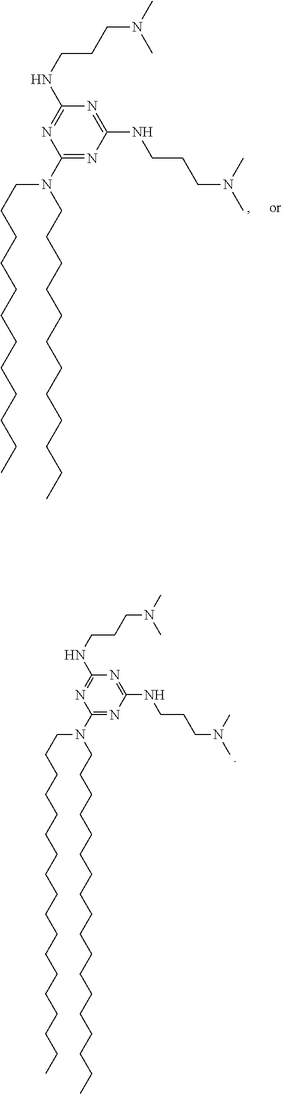 [SMILES: CCCCCCCCCCCCN(CCCCCCCCCCCC)c1nc(NCCCN(C)C)nc(NCCCN(C)C)n1 ; CCCCCCCCCCCCCCCCCCN(CCCCCCCCCCCCCCCCCC)c1nc(NCCCN(C)C)nc(NCCCN(C)C)n1]

In some embodiments, the each triazine lipid of the vector is

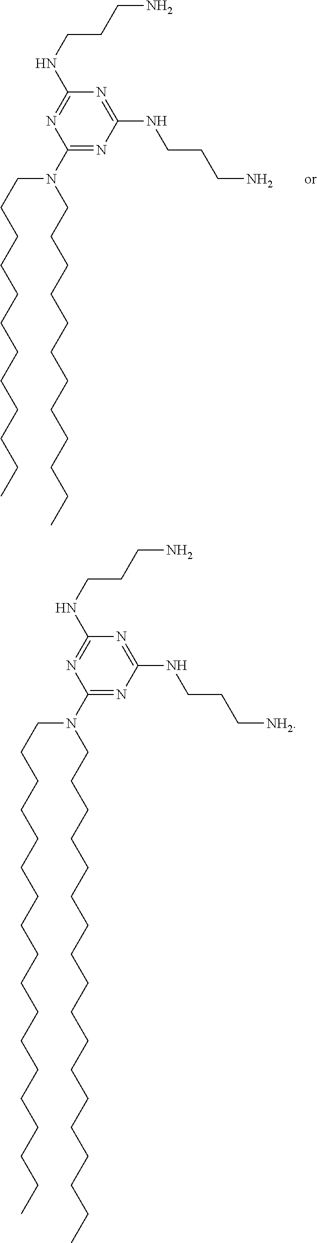 [SMILES: CCCCCCCCCCCCN(CCCCCCCCCCCC)c1nc(NCCCN)nc(NCCCN)n1 ; CCCCCCCCCCCCCCCCCCN(CCCCCCCCCCCCCCCCCC)c1nc(NCCCN)nc(NCCCN)n1]

The presently disclosed subject matter also includes solid-phase synthesis method for synthesizing triazine lipids, some or all of which, may be used as non-viral vectors for administration with an immunostimulatory antigen. In the method, a resin, such as an amine-reactive resin, is reacted with a first amine headgroup to generate an amine terminated resin. In some embodiments, the amine-reactive resin is 2-chlorotrityl chloride resin. A dichlorotriazine is then formulated substituting the amine terminated resin with a cyanuric chloride via nucleophilic aromatic substitution. The dichlorotriazine is then reacted with a lipid tail to form a monochlorotriazine, which is subsequently reacted with a second amine group to form the triazine lipid. After formation, the triazine lipid is cleaved from the resin. In some embodiments, the amine-reactive resin is 2-chlorotrityl chloride resin. In some embodiments, the lipid tail is a saturated or unsaturated dialkylamine. In one such embodiment, the lipid tail is selected from the group consisting of

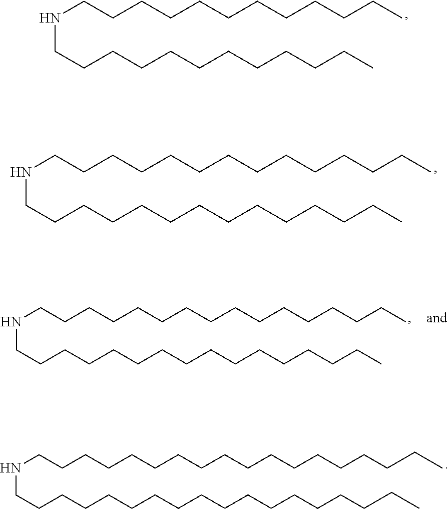 [SMILES: CCCCCCCCCCCCNCCCCCCCCCCCC ; CCCCCCCCCCCCCCNCCCCCCCCCCCCCC ; CCCCCCCCCCCCCCCCNCCCCCCCCCCCCCCCC ; CCCCCCCCCCCCCCCCCCNCCCCCCCCCCCCCCCCCC]

In some embodiments, the first amine headgroup is a diamine. In one such embodiment, the first amine headgroup is selected from the group consisting of

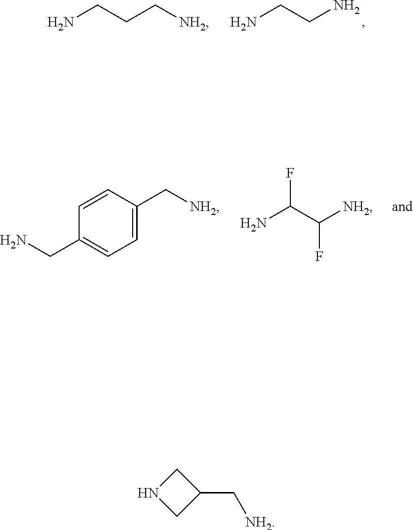

In some embodiments, the second amine headgroup is selected from the group consisting of

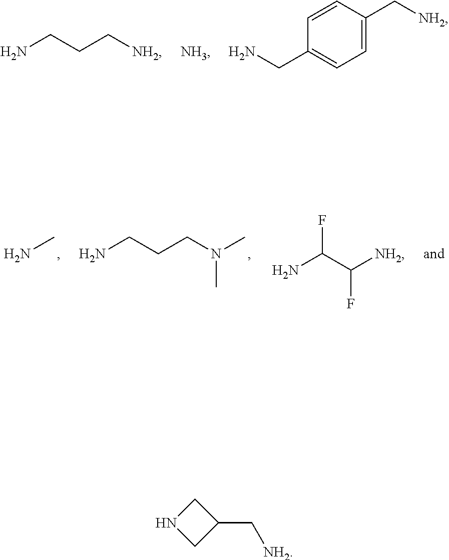

In some embodiments, the second amine headgroup is a diamine. It is appreciated, that the triazine lipids which can be synthesized employing the above-described method of synthesis is not necessarily limited limited to those including amine headgroups and tail groups expressly referred to above. Rather, alternative headgroups with at least one reactive nucleophilic amine which can be substituted with cyanuric chloride via nucleophilic aromatic substitution to yield a dichlorotriazine or added to a tailed monochlorotriazine in a manner consistent with that disclosed above may, in some embodiments, be alternatively utilized. Similarly, alternative lipid tails with at least one reactive nucleophilic amine which can be added to the dichlorotriazine in a manner consistent with that disclosed above may, in some embodiments, be alternatively utilized. For instance, in some embodiments, the lipid tail may contain primary or secondary amines and may be unsaturated and/or branched.

Certain triazine lipids disclosed herein have been found to inhibit canonical NF_{K}B transcriptional activity and thus may be utilized in non-viral transfection vectors for administration with an immunostimulatory antigen to reduce the reactogenic response invoked by the immunostimulatory antigen. Accordingly, in another aspect, the presently disclosed subject matter also includes methods for inhibiting canonical NF_{K}B transcriptional activity during an immune response to an immunostimulatory antigen within a subject. In some embodiments, the method includes administering a non-viral triazine lipid-based vector including a plurality of triazine lipids to a subject concurrently with an immunostimulatory antigen. In some embodiments, the immunostimulatory antigen is an immunogenic peptide. In some embodiments, the immunostimulatory antigen is an immunostimulatory nucleic acid. In some embodiments, each lipid of the triazine lipids is cationic. In some embodiments, the triazine lipid includes a lipid tail group, a triazine linker, and a cationic headgroup. In some embodiments, the lipid tail comprises a dialkylamine. In some embodiments, each triazine lipid of the non-viral vector is of the formula

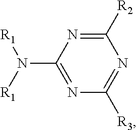

wherein: R₁ is alkyl; R₂ is

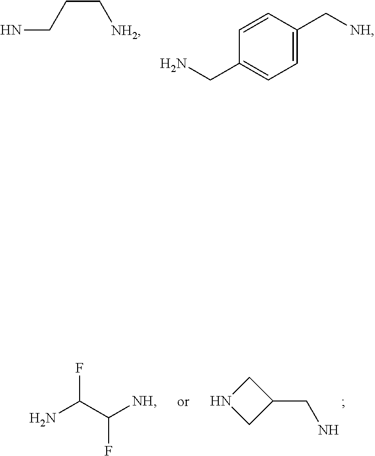

and R₃ is NH₂,

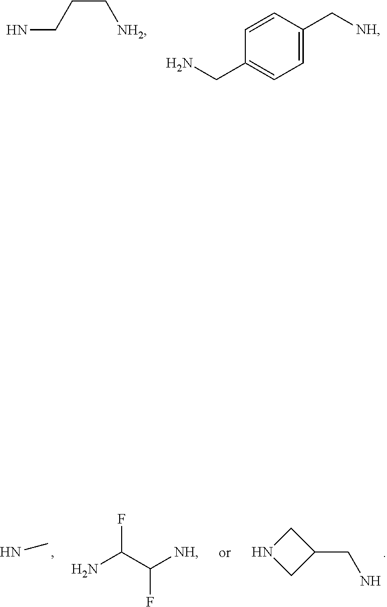

In some embodiments, R₁ comprises 18 or fewer alkyl carbons. In some embodiments, R₁ comprises at least 12 alkyl carbons. In some embodiments, wherein R₁ comprises 18 alkyl carbons. In some embodiments, R₁ comprises 12 alkyl carbons. In some embodiments, the non-viral triazine lipid-based vector is a liposome. Liposomes including triazine lipids disclosed herein may be formed by any suitable method. For example, in some embodiments, the liposomes may be formed by thin film hydration. In some embodiments, thin film hydration may be followed by sonication. In some embodiments, the non-viral triazine lipid-based vector is lipid nanoparticles. Lipid nanoparticle vectors including triazine lipids disclosed herein may be formed by any suitable method. For example, in some embodiments, lipid nanoparticle vectors may be formed via thin film hydration.

In some embodiments the non-viral triazine lipid-based vector administered to the subject in the method may include one or more additional lipids selected from the group consisting of dioleoylphosphatidylethanolamine (DOPE), distearolyphosphatidycholine (DSPC), and 1, 2-distearoyl-sn-glycero-3-phsphoethanolamine-polyethylene glycol (DSPE-PEG). In one such embodiment, the non-viral triazine lipid-based vector includes DSPE-PEG (550-2000). In some embodiments, the non-triazine lipid-based vector administered in the method may stimulate non-canonical NF_{K}B transcriptional activity. In one such embodiment, each triazine lipid of the plurality of triazine lipids is

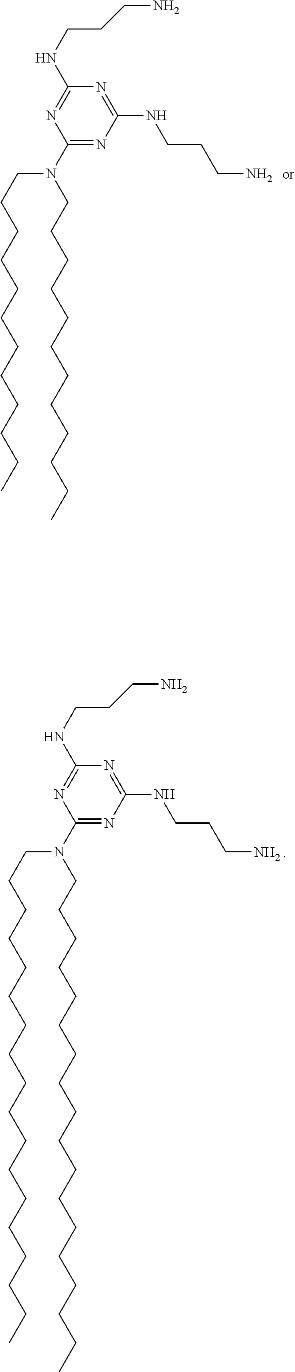 [SMILES: CCCCCCCCCCCCN(CCCCCCCCCCCC)c1nc(NCCCN)nc(NCCCN)n1 ; CCCCCCCCCCCCCCCCCCN(CCCCCCCCCCCCCCCCCC)c1nc(NCCCN)nc(NCCCN)n1]

In some embodiments, the immunostimulatory antigen is an immunogenic polypeptide and the non-viral triazine-based vector invokes an anti-polypeptide response which is greater than an anti-polypeptide response invoked by dioleoyl-3-trimethylammonium propone (DOTAP) or 1,2-Dimyristoyl-sn-glycero-3-phosphocholine (DMPC) when administered concurrently with the immunogenic polypeptide. In one such embodiment, the anti-polypeptide response with the non-viral triazine lipid-based vector is greater than the anti-polypeptide response invoked by DOTAP or DMPC. In some embodiments, the anti-polypeptide response with the non-viral triazine lipid-based vector is at least 300-fold greater relative to the anti-polypeptide response with DOTAP. In some embodiments, the non-viral triazine lipid-based vector and the immunostimulatory antigen may be administered together as an immunogenic composition including the non-viral triazine lipid-based vector and the immunostimulatory antigen. In one such embodiment, the non-viral triazine lipid-based vector and the immunostimulatory antigen are administered with a pharmaceutically-acceptable carrier.

Certain triazine lipids described herein may also find utility in modulating NF_{K}B pathways in vitro. In this regard, the presently disclosed subject matter also includes a method for inhibiting canonical transcriptional activity during an immune response within one or more cells. In the method, the one or more cells are contacted with one or more triazine lipids. In some embodiments, each triazine lipid of the one or more triazine lipids is cationic. Each triazine lipid of the one or more triazine lipids may be of the formulation and include the feature specified for the various embodiments of the triazine lipids of the triazine lipid-based vector described above with respect to the methods for inhibiting canonical NF_{K}B transcriptional activity during an immune response to an immunostimulatory antigen within a subject.

While there are limited reports of NF_{K}B modulation with lipid-based materials, prior reports indicate activity that might include NF_{K}B modulation, although not experimentally determined. In this regard, Vangasseri, et al.¹⁷⁵ utilized CD80 expression as one marker of activity in cells treated with DOTAP. In that case, CD80 expression was enhanced with increasing concentrations of DOTAP, which Vangasseri, et al. attribute to TLR9 signaling and canonical NF_{K}B stimulation, but may actually be the result of non-canonical NF_{K}B activation. Based on this observation, Vangasseri, et al. demonstrate that anionic lipids (e.g. PA, PG) and zwitterionic lipids (e.g. PC) do not increase expression of CD80, but zwitterionic lipids that are capped with an ethyl group on the phosphate rendering them cationic (e.g. EPC) result in increased CD80 expression. Subsequently, Vangasseri, et al. demonstrate that lipids with transition temperatures below 50° C. (e.g. DM, DO) exhibit increased CD80 expression relative to their high Tm counterparts (e.g. DS, DP). Therefore, based on this observation there is an indication of promiscuity in the cationic lipid head group for inclusion in the lipid, while lipid tails shorter than C16, and those with unsaturation, or branching that result in reduced transition temperature will have improved activity. Accordingly, while the headgroups of the triazine lipids utilized in the vectors and methods disclosed herein may sometimes be referred to as including a specific amine headgroup or selected from a particular grouping of candidate amine headgroups and/or including a specific saturated, unbranced tail or selected from a particular grouping of saturated tails, unbranched tails, one of ordinary skill in the art will appreciate that triazine lipids including alternative cationic lipid headgroups and/or unsaturated or branching tails which cause the triazine lipid to exhibit a transition temperature below 50° C. can, in some embodiments, be alternatively used in the vectors and method disclosed herein.

The presently-disclosed subject matter is illustrated by specific but non-limiting examples throughout this description. The examples may include compilations of data that are representative of data gathered at various times during the course of development and experimentation related to the present invention(s). Each example is provided by way of explanation of the present disclosure and is not a limitation thereon. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

### EXAMPLES

### Example 1: In Vitro Reagents Synthesized Using Cyanuric Chloride

A library of triazine (TZ) based lipids was synthesized using cyanuric chloride as a linker, dialkylamines as lipid tails and various small molecules to generate diverse head groups. The headgroups were chosen due to their cost effectiveness, commercial availability, and diversity in functional moieties, which provide a platform for future evaluation of an expanded library of triazine based lipids.

### Methods

### Materials and Instrumentation

Beta-alanine-tert-butyl ester, cyanuric chloride, didodecyl-amine, diisopropylethylamine (DIPEA), 2-mercaptoethylamine HCl, morpholine, ninhydrin, N,N-dimethyl diaminopropane and trityl chloride were purchased from TCI America (Portland, OR). Dioctadecylamine was purchased from Sigma-Aldrich (Milwaukee, WI). N-Boc-1,3-diaminopropane was purchased from Matrix Scientific (Columbia, SC). 1,2-Dimyristoyl-sn-glycero-3-phosphocholine (DMPC), 1,2-distearoyl-sn-glycero-3-phosphocholine (DSPC), 1,2-dimyristoyl-sn-glycero-3-phospho-(10-rac-glycerol) (DMPG), 1,2-dioleoyl-sn-glycero-3-phosphoethanolamine (DOPE), 1,2-di-O-octadecenyl-3-trimethylammonium propane (DOTMA) and cholesterol were purchased from Avanti Polar Lipids, Inc. (Alabaster, AL). Solvents for reactions were purchased from various suppliers through VWR (Radnor, PA). Thin layer chromatography (TLC; Millipore Sigma, Silica gel 60 F254) was visualized under UV light or with 2% ninhydrin in DMSO. Final compound purity was assessed via a Waters 2707 Autosampler, Waters 2545 Quaternary Gradient Module pump and Waters 2998 Photodiode Array Detector following injection into a Waters XBridge C18 3.5 mm column (part no. 186003034) using a water, acetonitrile and methanol mixture as described in the figures below and detected at 205 and 254 nm. ¹H and ¹³C NMR spectra were recorded in deuterated chloroform using a Varian 400 MHz or Varian 500 MHz spectrometer equipped with a 5 mm OneProbe (Cambridge Isotope Laboratories, Inc.; Tewksbury, MA). HR-MS was performed on an Agilent 6230B TOF LC/MS instrument in positive ion by direct injection of the compounds. Lipopeptide purification was performed using the Waters system described above.

Cell strains. LD50 and cytotoxicity assays were performed on bone marrow derived macrophages from C57BL/6J mice. Plasmid transfections were carried out using HeLa cells or HEK-293 cells.

Mice. Six-week-old female C57BL/6J mice were purchased from The Jackson Laboratory (Bar Harbor, ME) and housed in a specific pathogen-free facility at the University of Kentucky. Mice were sedated for immunization and blood collection with isoflurane gas. Blood was collected by superficial temporal vein puncture using a small animal lancet (Medipoint) into a microcentrifuge tube and centrifuged for 10 min at 2000×g after standing at 4° C. for 1 h. Serum was stored at −80° C. for later antibody detection. All procedures were approved by the University of Kentucky Institutional Animal Care and Use Committee.

### Synthesis

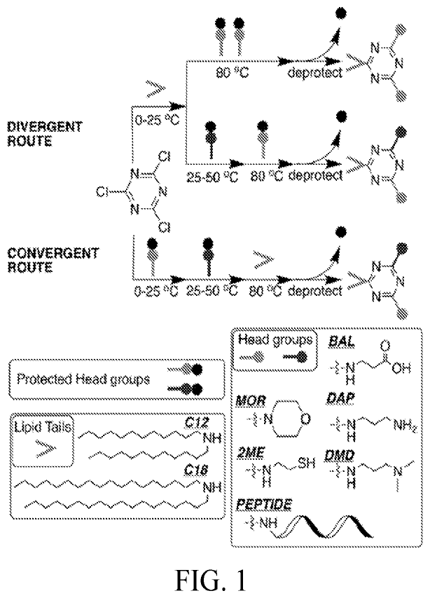

Two approaches were taken for the synthesis of the TZ lipids: a convergent and a divergent route (FIG. 1). In the convergent approach, two small molecule nucleophiles with protected, ionizable moieties were reacted with cyanuric chloride through nucleophilic aromatic substitution (NAS). The resulting monochlorotriazine was then reacted with a long-chained secondary amine lipid tail (dio-ctadecylamine or didodecylamine) to yield the final protected lipid. In the divergent approach, the lipid tail was reacted first to form a dichloro-triazine, followed by headgroup diversification through addition of various nucleophilic small molecule moieties as headgroups. In both approaches, the first NAS was initiated on ice and allowed to stir at room temperature in chloroform for at least 4 hours. The second substituent was added at room temperature in chloroform and heated to 50° C. for at least 24 hours. The final NAS reaction was performed in xylenes or dioxane and heated from room temperature to 80° C. for at least 72 hours. In each reaction, excess nucleophile or DIPEA served as base. The reactions were monitored at each step via thin layer chromatography and characterized by nuclear magnetic resonance and mass spectrometry. Small molecule nucleophiles with reactant pendant moieties were protected with acid labile protecting groups and deprotected as the final step in the lipid synthesis with trifluoroacetic acid in dichloromethane.

Intermediate A. Intermediate A was prepared by adding 1 equiv. of cyanuric chloride to a stirring solution of chloroform with 2.4 equiv. of beta-alanine-tert-butyl ester and 10 equiv. of DIPEA on ice. The mixture was allowed to come to room temperature, then heated overnight at 50° C. Remaining beta-alanine-tert-butyl ester was removed by washing the dried product three times with brine. The monochlorotriazine was purified using a 0-30% ethyl acetate/CH2Cl2 mixture on silica gel and the final product was eluted from the column using ethyl acetate, which was evaporated to yield intermediate A (73.8%) (30% ethyl acetate:chloroform, Rf=0.88). ¹H-NMR (500 MHZ, CHCl3) d 5.65-5.86 (m, 2NH), 3.56-3.68 (m, 4H), 2.47-2.52 (m, 4H), 1.42-1.48 (m, 18H); 13C-NMR (125 MHz, CHCl3) d 171.18, 165.57, 156.38, 81.13, 36.58, 34.94, 28.07; HRMS MW calculated for C17H28ClN5O4 (M+H)+: =402.1903; found: 402.1939.

Intermediate B. Intermediate B was prepared by adding 1 equiv. of cyanuric chloride to a stirring solution of chloroform with 2.4 equiv. of N-Boc-1,3-diaminopropane and 10 equiv. of DIPEA on ice. The mixture was allowed to come to room temperature, then heated overnight at 50° C. Remaining N-Boc-1,3-diaminopropane was removed by washing the dried product three times with brine. The monochlorotriazine was purified using a 0-30% ethyl acetate/CH2Cl2 mixture on silica gel and the final product was eluted from the column using ethyl acetate, which was evaporated to yield intermediate B (86%) (50% ethyl acetate:chloroform, Rf=0.51). 1H-NMR (500 MHz, CHCl3) d 4.96-6.51 (m, 4NH), 3.38-3.49 (m, 4H), 3.19 (m, 4H), 1.74 (m, 4H), 1.44 (m, 18H); 13C-NMR (125 MHz, CHCl3) d 168.02, 165.77, 156.17, 79.23, 37.97, 37.56, 30.04, 28.39; HRMS MW calculated for C19H34ClN704 (M+H)+: =460.2434; found: 460.2505.

2-[(Triphenylmethyl)thio]ethanamine. 2-[(Triphenylmethyl)thio]ethanamine (CAS number: 1095-85-8) was prepared by an adaptation of the procedure described by Watrelot et al.¹⁰⁷ To a stirred solution of 2-mercaptoethylamine HCl (1.1 equiv.) in dichloromethane at 0° C. under nitrogen was added dropwise trifluoracetic acid (TFA, 3 mL) followed by dropwise addition of trityl chloride (1 equiv.). The reaction was stirred for 2.5 hours at 0° C. then concentrated and diluted in CHCl3 (10 mL) and washed 3 times with 1 M NaOH and once with brine. The organic layer was then dried over magnesium sulfate and filtered and evaporated to dryness to afford the desired compound (92%) without further purification. 1H-NMR (500 MHz, CDCl3) d 7.43 (m, 6H), 7.28 (m, 6H), 7.21 (m, 3H), 2.6 (t, J=6.5 Hz, 2H), 2.32 (t, J=6.5 Hz, 2H), 1.21 (bs, 2H, NH2); 13C-NMR (125 MHz, CDCl₃) δ 144.87, 129.56, 127.82, 126.61, 66.50, 41.08, 36.35.

Intermediate C. Intermediate C was prepared by adding 1 equiv. of cyanuric chloride to a stirring solution of chloroform with 1.2 equiv. of beta-alanine-tert-butyl ester and 10 equiv. of DIPEA on ice. The mixture was allowed to come to room temperature and reacted for 4 hours until the disappearance of cyanuric chloride was confirmed on TLC (chloroform, Rf=0.58). To this mixture 1.1-1.5 equiv. of 2-[(triphenylmethyl)thio]ethanamine was added and stirred at room temperature for 24 hours. The final compound was dried and dissolved in ethyl acetate and then purified by washing with 0.5 M HCl three times then twice with brine. The organic phase was dried over magnesium sulfate and evaporated to yield intermediate C (97.7-99.3%). Of note, the formation of this product starting with 2-[(triphenylmethyl)thio]ethanamine yields an insoluble white solid following the addition of 2-[(triphenylmethyl)thio]ethanamine, which is extremely difficult to purify and dissolve for further reactions. 1H-NMR (500 MHZ, CHCl3) d 7.39 (m, 6H), 7.16-7.28 (m, 9H), 5.67-6.14 (m, 2NH), 3.51-3.66 (m, 2H), 3.14-3.30 (m, 2H), 2.39-2.50 (m, H4), 1.42-1.47 (m, 9H); 13C-NMR (125 MHz, CHCl3) d 171.20, 168.31, 165.39, 146.84, 144.67, 129.45, 127.85, 127.17, 126.69, 81.09, 66.73, 39.60, 36.43, 34.86, 31.40, 28.07; HRMS MW calculated for C31H34ClN5O4S (M+H)+: =576.2195; found: 576.2198.

Intermediate D. Intermediate D was prepared by adding 1.1-1.5 equiv. of cyanuric chloride to a solution of chloroform with 1 equiv. of dioctadecylamine and 10 equiv. of DIPEA. The solution was started at −78° C. and allowed to come to 4° C. overnight. In the morning the reaction was assessed for the disappearance of the secondary amine using 2% ninhydrin in DMSO on TLC (3:2 CH2Cl2:hexanes, Rf=0.95). The completed reaction was dried by rotary evaporation, then precipitated from chloroform with MeOH and filtered. This process was repeated twice, and the resulting white powder was resuspended in CHCl3, dried over magnesium sulfate, filtered and evaporated to dryness to afford intermediate D (92-95%). 1H-NMR (500 MHz, CHCl3) d 3.51 (t, J=10, 4H), 1.55-1.61 (m, 4H), 1.20-1.32 (m, 60H), 0.86 (t, J=10, 6H); 13C-NMR (125 MHz, CHCl3) d 169.66, 164.16, 47.75, 31.84, 29.61, 29.58, 29.57, 29.55, 29.49, 29.40, 29.28, 29.15, 27.06, 26.59, 22.60, 14.03; HRMS MW calculated for C39H74Cl2N4 (M+H)⁺: =669.5363; found: 669.5361.

Intermediate E. Intermediate E was prepared by adding 1.2 equiv. of beta-alanine-tert-butyl ester to a solution of chloroform with 1 equiv. of intermediate D and 10 equiv. of DIPEA. The mixture was stirred at room temperature for 2 hours then heated to 50° C. and allowed to react overnight. Remaining beta-alanine-tert-butyl ester was removed by washing the reaction mixture three times with brine. The compound was further purified on a silica gel column using a 10% ethyl acetate/chloroform mixture to yield intermediate E (51.2%) (CHCl3, Rf=0.50). 1H-NMR (500 MHz, CHCl3) d 5.52-5.6 (m, NH), 3.57-3.66 (m, 2H), 3.35-3.51 (m, 4H), 2.49 (t, J=7.5, 2H), 1.52-1.62 (m, 4H), 1.44 (s, 9H), 1.20-1.32 (m, 60H), 0.88 (t, J=7.5, 6H); 13C-NMR (125 MHz, CHCl3) d 171.13, 168.61, 165.12, 164.44, 80.90, 47.33, 47.09, 35.00, 31.83, 29.61, 29.57, 29.54, 29.49, 29.37, 29.30, 29.27, 28.02, 27.73, 26.97, 26.70, 22.60, 14.03; HRMS MW calculated for C46H88ClN5O2 (M+H)+: =778.6699; found: 778.6692.

Intermediate F. Intermediate F was prepared in the same manner as intermediate D using didodecylamine with a similar product yield (93-95%). 1H-NMR (500 MHz, CHCl3) d 3.51 (t, J=9.5, 4H), 1.54-1.62 (m, 4H), 1.22-1.32 (m, 36H), 0.86 (t, J=8.5, 6H); 13C-NMR (125 MHz, CHCl3) d 169.60, 164.08, 47.71, 31.86, 29.55, 29.26, 29.14, 27.02, 26.56, 22.63, 14.07; HRMS MW calculated for C27H50Cl2N4 (M+H)+: =501.3485; found: 501.3489.

Intermediate G. Intermediate G was prepared in the same manner as described for intermediate E using intermediate F with a similar product yield (50.4%). 1H-NMR (500 MHZ, CHCl3) d 5.52-5.6 (t, J=6.1, NH), 3.58-3.62 (m, 2H), 3.35-3.49 (m, 4H), 2.49 (t, J=6.5, 2H), 1.50-1.61 (m, 4H), 1.42 (s, 9H), 1.20-1.29 (m, 36H), 0.86 (t, J=7, 6H); 13C-NMR (125 MHZ, CHCl3) d 171.03, 168.45, 165.11, 164.38, 80.67, 47.28, 47.01, 35.02, 31.78, 29.52, 29.49, 29.48, 29.43, 29.33, 29.24, 29.21, 27.95, 27.69, 26.92, 26.64, 22.54, 13.97; HRMS MW calculated for C34H64ClN5O2 (M+H)+: =610.4821; found: 610.4842.

Lipid 1. Lipid 1 was prepared by adding 1 equiv. of didodecylamine to a stirring solution of dioxane containing 2 equiv. of intermediate A and 10 equiv. of DIPEA. The solution was heated to 80° C. After at least 48 hours (shorter reaction periods led to reduction in product yield) the reaction was evaporated using a rotary evaporator and re-dissolved in chloroform then washed three times with brine. The organic phase was then dried over magnesium sulfate, filtered, and dried in a rotary evaporator. The resulting solid was purified on a silica gel column using a chloroform to ethyl acetate mobile phase gradient (1:9 ethyl acetate:chloroform Rf=0.5) and confirmed on NMR before being deprotected using a mixture of 1:1 TFA and dichloromethane and evaporated to dryness to yield lipid 1 (39.7-52.7%, final product). 1H-NMR (500 MHZ, CHCl3) ö 8.30 (s, 2OH), 3.60-3.70 (m, 4H), 3.44-3.54 (t, J=7.5, 4H), 2.63 (t, J=5, 4H), 1.56-1.64 (m, 4H), 1.23-1.32 (m, 36H), 0.87 (t, J=5, 6H); 13C-NMR (125 MHZ, CHCl3) ö 196.46, 175.78, 161.64, 154.61, 107.24, 48.06, 36.65, 33.71, 31.82, 29.57, 29.55, 29.53, 29.29, 29.26, 27.77, 26.95, 22.59, 14.02; HRMS MW calculated for C33H62N6O4 (M+H)+: =607.4905; found: 697.4904.

Lipid 2. Lipid 2 was prepared in the same manner as compound 1 using dioctadecylamine and yielded compound 2 (21.7-27.6%, final product). 1H-NMR (400 MHz, CHCl3) ö 8.18 (s, 2COOH), 3.39-3.74 (m, 8H), 2.53-2.79 (m, 4H), 1.52-1.64 (m, 4H), 1.18-1.33 (m, 60H), 0.86 (t, J=6, 6H); 13C-NMR (100 MHZ, CHCl3) ö 175.64, 161.67, 154.74, 48.24, 36.62, 33.44, 31.89, 29.68, 29.63, 29.60, 29.58, 29.37, 29.33, 27.84, 27.01, 22.66, 14.08; HRMS MW calculated for C45H86N6O4 (M+H)+: =775.6783; found: 775.6790.

Lipid 3. Lipid 3 was prepared by adding 1 equiv. of didodecylamine to a stirring solution of dioxane containing 2 equiv. of intermediate B and 10 equiv. of DIPEA. The solution was heated to 80° C. After at least 48 hours (shorter reaction periods led to reduction in product yield) the reaction was evaporated and dissolved in chloroform then washed three times with brine. The organic phase was then dried over magnesium sulfate, filtered, and dried using a rotary evaporator. The resulting solid was purified on a silica gel column using a chloroform to ethyl acetate mobile phase gradient (ethyl acetate Rf=0.46) and confirmed on NMR before being deprotected using a mixture of 1:1 TFA in dichloromethane and evaporated to dryness to yield lipid 3 (32-46.0%, final product). 1H-NMR (500 MHZ, CHCl3) ö 3.28-3.48 (m, 8H), 2.77 (t, J=7.5, 4H), 1.68 (t, J=7.5, 4H), 1.48-1.58 (m, 4H), 1.16-1.32 (m, 36H), 0.86 (t, J=7.5, 6H); 13C-NMR (125 MHz, CHCl3) ö 164.88, 46.71, 31.90, 29.67, 29.65, 29.63, 29.53, 29.34, 28.04, 27.11, 22.66, 14.10; HRMS MW calculated for C33H68N8 (M+H)⁺: =557.6540; found: 577.5639.

Lipid 4. Lipid 4 was prepared in the same manner as compound 3 using dioctadecylamine and yielded (55.8-56%, final product). 1H-NMR (500 MHZ, CHCl3) d 3.31-3.49 (m, 8H), 2.82-3.04 (m, 4H), 1.72-1.92 (m, 4H), 1.48-1.58 (m, 4H), 1.17-1.34 (m, 60H), 0.86 (t, J=7.5, 6H); 13C-NMR (125 MHz, CHCl3) d 164.54, 46.78, 31.83, 29.62, 29.56, 29.45, 29.26, 27.93, 27.05, 22.59, 14.02; HRMS MW calculated for C45H92N8 (M+H)+: =745.7518; found: 745.7526. Of note, the peak resolution of this compound was poor and while several attempts were made to improve the quality of the spectra using various solvents alone and in combination, as well as various additives, the definition could not be improved beyond that presented here.

Lipid 5. Lipid 5 was prepared by adding 1 equiv. of didodecylamine to a stirring solution of dioxane containing 2 equiv. of intermediate C and 10 equiv. DIPEA. The solution was heated to 80° C. After at least 48 hours (shorter reaction periods led to reduction in product yield) the reaction was concentrated by rotary evaporation and re-dissolved in chloroform then washed three times with brine. The organic phase was then dried over magnesium sulfate, filtered, and dried using a rotary evaporator. The resulting solid was deprotected using a mixture of 1:1 TFA in dichloromethane and evaporated to dryness. The resulting solid was purified by silica gel chromatography by first eluting impurities with chloroform and ethyl acetate, then eluting the final product with methanol. The methanol fraction was dried and re-dissolved in chloroform before being filtered over magnesium sulfate to yield lipid 5 (90.6%, final product). 1H-NMR (500 MHz, CHCl3) d 8.40 (OH), 7.67 (s, NH), 3.47-3.70 (m, 8H), 2.62-2.75 (m, 4H), 1.55-1.66 (m, 4H), 1.42 (t, J=8.6, SH), 1.231.33 (m, 36H), 0.87 (t, J=7, 6H); 13C-NMR (125 MHz, CHCl3) d 176.27, 162.51, 161.61, 154.95, 154.41, 117.19, 114.89, 93.02, 48.19 43.93, 36.35, 33.06, 31.81, 30.91, 29.53, 29.52, 29.35, 29.29, 29.24, 27.83, 27.77, 27.61, 26.99, 26.93, 23.31, 22.58, 14.00; HRMS MW calculated for C32H62N602S (M+H)+: =595.4728; found: 595.4735.

Lipid 6. Lipid 6 was prepared in the same manner as compound 5 using dioctadecylamine and yielded lipid 6 (72.6%, final product). 1H-NMR (500 MHZ, CHCl3) d 9.01 (s, OH), 7.78 (s, NH), 3.44-3.73 (m, 8H), 2.65-2.74 (m, 4H), 1.53-1.65 (m, 4H), 1.42 (t, J=8.6, SH), 1.22-1.31 (m, 60H), 0.86 (t, J=7, 6H); 13C-NMR (125 MHz, CHCl3) d 175.46, 162.97, 161.61, 155.09, 154.54, 117.59, 114.71, 48.32, 44.02, 36.83, 33.24, 30.89, 31.01, 29.68, 29.63, 29.59, 29.43, 29.37, 29.33, 27.91, 27.07, 27.02, 23.41, 22.66, 14.08, 13.08; HRMS MW calculated for C44H86N602S (M+H)+: =763.6606; found: 763.6604.

Lipid 7. Lipid 7 was prepared by adding 8 equiv. of morpholine to 1 equiv. of intermediate D dissolved in chloroform and refluxed overnight. After 48 hours, the reaction was first washed with 0.5 M NaOH, then brine and the organic phase was evaporated to yield lipid 7 (99.3%, final product) (ethyl acetate, Rf=0.75). 1H-NMR (500 MHz, CHCl3) d 3.67-3.75 (m, 16H), 3.44 (t, J=7.5, 4H), 1.50-1.57 (m, 4H), 1.22-1.32 (m, 60H), 0.88 (t, J=7.5, 6H); 13C-NMR (125 MHz, CHCl3) d 165.34, 164.96, 66.84, 46.74, 43.55, 31.84, 29.62, 29.60, 29.58, 29.57, 29.56, 29.42, 29.27, 27.84, 27.01, 22.60, 14.03; HRMS MW calculated for C47H90N6O2 (M+H)+: =771.7198; found: 771.7197.

Lipid 8. Lipid 8 was prepared by adding 1 equiv. of intermediate E to 8 equiv. of morpholine in xylenes or dioxane and heating to 80° C. for 48 hours. The solvent was removed using a rotary evaporator at 80-90° C. and the resulting solid was dissolved in chloroform and washed three times with 0.5 M HCl then twice with brine. The organic phase contained a number of impurities and was purified by silica gel chromatography using at 0-10% ethyl acetate:chloroform mobile phase gradient. The pure product was then confirmed on NMR before being deprotected using a mixture of 1:1 TFA in dichloromethane and evaporated to dryness to yield lipid 8 (86.6%, final product). 1H-NMR (500 MHZ, CHCl3) ö 8.23 (m, OH), 3.66-3.88 (m, 10H), 3.32-3.52 (m, 4H), 2.57-2.75 (m, 2H), 1.50-1.62 (m, 4H), 1.22-1.32 (m, 60H), 0.86 (t, J=8, 6H); 13C-NMR (125 MHz, CHCl3) ö 171.88, 166.05, 165.33, 165.00, 80.95, 66.91, 46.80, 43.56, 36.48, 35.77, 31.90, 29.68, 29.65, 29.64, 29.64, 27.52, 29.34, 28.13, 27.09, 22.67, 14.09; HRMS MW calculated for C46H88N6O3 (M+H)+: =773.6991; found: 773.6991.

Lipid 9. Lipid 9 was prepared by adding 20 equiv. of N,N-dimethyl-1,3-diaminopropane to a stirring solution of intermediate F and 10 equiv. of DIPEA in dioxane. The reaction was allowed to stir at room temperature for 24 hours then heated at 80° C. for another 48 hours. The reaction was then concentrated using a rotary evaporator and the product was dissolved in ethyl acetate and washed three times with brine. The organic phase was collected, dried over magnesium sulfate and concentrated to yield lipid 9 (92.3%, final product). 1H-NMR (500 MHz, CHCl3) ö 5.15 (s, 2NH), 3.36-3.49 (m, 4H), 3.28-3.36 (m, 4H), 2.27 (t, J=9.6, 4H), 2.16 (s, 12H), 1.59-1.76 (m, 4H), 1.45-1.57 (m, 4H), 1.17-1.28 (m, 36H), 0.83 (t, J=8.6, 6H); 13C-NMR (125 MHz, CHCl3) ö 165.90, 164.89, 57.63, 46.71, 45.44, 39.17, 31.83, 29.62, 29.61, 29.58, 29.56, 29.46, 29.27, 27.99, 27.72, 27.05, 22.59, 14.02; HRMS MW calculated for C37H76N8 (M+H)+: =633.6266; found: 633.6270.

Lipid 10. Lipid 10 was prepared in the same manner as compound 9 using intermediate D and yielded lipid 10 (93.4%, final product). 1H-NMR (500 MHz, CHCl3) ö 5.20 (s, 2NH), 3.30-3.50 (m, 8H), 2.35 (t, J=8.4, 4H), 2.22 (s, 12H), 1.64-1.78 (m, 4H), 1.46-1.59 (m, 4H), 1.18-1.32 (m, 60H), 0.83 (t, J=8.6, 6H); 13C-NMR (125 MHz, CHCl3) ö 165.42, 164.73, 57.63, 46.79, 45.37, 39.23, 31.89, 29.68, 29.53, 29.33, 28.03, 27.55, 27.11, 22.66, 14.09; HRMS MW calculated for C49H101N8 (M+H)+: =801.8144; found: 801.8126.

Lipid 11. Lipid 11 was prepared by adding 4-8 equiv. of N-Boc-1,3-diaminopropane to a stirring solution of dioxane containing 1 equiv. of intermediate G and 10 equiv. of DIPEA. The solution was stirred at 80° C. for 72 hours after which the solvent was removed using a rotary evaporator. The resulting solid was then dissolved in chloroform and washed three times with 0.5 M HCl then twice with brine. The organic phase was dried then purified by silica gel chromatography using a chloroform to ethyl acetate gradient and the product was confirmed on NMR before being deprotected using a mixture of 1:1 TFA in dichloromethane and evaporated to dryness to yield pure lipid 11 (90.3%, final product). 1H-NMR (500 MHz, CHCl3) d 7.98 (s, 3NH), 7.66 (s, OH), 3.38-3.69 (m, 8H), 2.95-3.13 (m, 2H), 2.54-2.69 (m, 2H), 1.92-2.09 (m, 2H), 1.51-1.64 (m, 4H), 1.22-1.32 (m, 36H), 0.87 (t, J=5, 6H); 13C-NMR (125 MHZ, CHCl3) d 175.48, 154.54, 48.23, 31.82, 29.53, 29.26, 27.73, 27.60, 26.94, 22.59, 14.00; HRMS MW calculated for C33H65N7O2 (M+H)+: =592.5273; found: 592.5277.

Lipid 12. Lipid 12 was prepared in the same manner as compound 11 using intermediate E and yielded lipid 12 (44.4%, final product). 1H-NMR (500 MHZ, CHCl3) d 7.92 (s, 3NH), 7.64 (s, OH), 3.30-3.72 (m, 8H), 2.92-3.20 (m, 2H), 2.51-2.72 (m, 2H), 1.89-2.15 (m, 2H), 1.53-1.63 (m, 4H), 1.20-1.34 (m, 60H), 0.87 (t, J=7.5, 6H); 13C-NMR (125 MHZ, CHCl3) d 175.51, 154.49, 48.18, 31.84, 29.63, 29.28, 27.73, 26.95, 24.78, 22.60, 14.02; HRMS MW calculated for C45H89N7O2 (M+H)+: =760.7151; found: 760.7159.

Lipopeptide Synthesis. Lipidation of an ApoA-I peptide spanning the residues 141-184 of the mouse sequence (ApoA-1141-184) was completed using intermediate D (C18 TZ linker). Resin was added to a vial, based on 22-40 mg of resin-cleaved and deprotected peptide (sequence BAGGLSPVAEEFRDRMRTHVDSLRTQLAPHSEQMRESLAQRLAELKSN) (Elim Biopharm, Inc.).

### Biophysical Characterization of Lipids and Nanoparticles

Formulation of lipid nanoparticles using triazine lipids. Lipid nanoparticles for all experiments were formed by thin lipid film hydration. For this procedure, the triazine lipids dissolved in chloroform were transferred in sufficient quantities (based on the desired final concentration and volume) into a sterile round bottom tube alone or in combination with other lipids (DOPE, DSPC, cholesterol, etc.). The organic solvents were to form a thin lipid film and dried overnight under vacuum. The lipids were then rehydrated in 20 mM HEPES solution and sonicated at 60° C. until opalescent (˜30 min).

Differential Scanning calorimetery. The transition temperature (Tₘ) of the lipids was determined using a multicell differential scanning calorimeter (TA Instruments). Liposomes were made with triazine lipids at a concentration of 10 mM in 20 mM HEPES buffer. These were heated to 60° C. and sonicated until the solution was translucent. For Tₘ determination, 250 μL of the liposome solution was transferred into reusable hastelloy ampoules while 250 μL of the HEPES solution was transferred to the third ampoule, leaving the reference ampoule empty. For lipids 7 and 8, which failed to form nanoparticles, 250 μL of the solution containing the lipid aggregate were transferred to the ampoules after sonication. Data was collected over a range of 10-110° C. at a rate of 2° C. min-1 in a heat-cool-heat cycle. After the run was complete, the CpCalc 2.1 software package was used to convert the raw data into a molar heat capacity and the data from the second heating cycle were processed using Microsoft Excel.

Size and Charge Determination. The size and charge of the nanoparticles were determined using a Zetasizer Nano ZS (Malvern Panalytical). For each lipid, two separate formulations of liposomes were tested at a concentration of 1 mM. The size was determined in ZEN0400 cuvettes using the following settings: four measurements of 15 five second runs detected at a backscatter angle of 173° at 25° C. The zeta potential for the liposomes was determined in a DTS1070 folded capillary zeta cell using the following settings; four measurements of at least 50 runs modelled with the Smoluchowski equation at 25° C. using the automatic settings from the instrument.

Carboxyfluorescein Encapsulation Assay. The ability of CC lipids to encapsulate molecules was tested using 5-(6)-carboxy-fluorescein (CF) purchased from Acros Organics (Pittsburgh, PA), which was purified using the protocol established by Ralston et al.¹⁷ Briefly, unpurified CF was dissolved in refluxing ethanol for 3 hours in the presence of activated charcoal and filtered. The filtrate was diluted in enough distilled water to achieve a 1:2 ethanol/water ratio and crystalized at 20° C. The crystalized CF was filtered and washed multiple times with distilled water and dried overnight. Solid CF was then dissolved in water and 5 M NaOH to a concentration of 250 mM and passed over an LH-20 Sephadex column. Five mL were purified on a 10×2 cm column by elution at room temperature with distilled water. CF eluted as a dark orange-red band that was quantified via absorbance at 492 nm using a coefficient of 6-CF (6 900 M cm⁻¹) as described by Weinstein et al. 18 For the encapsulation assay, thin lipid films of CC lipids were prepared as described above. After evaporating remaining organic solvent overnight, the lipids were resuspended in a solution of 200 mM CF. Control phosphatidylcholine liposomes were then purified using a PD10 desalting column (GE Life Sciences).

Determination of Nanoparticle pKa via TNS Fluorescence. Cationic liposome pKa was determined by measuring the fluorescence of 2-(p-toluidino)-6-napthalene sulfonic acid (TNS), as described by Jayaraman, et al.¹⁹ For this, liposomes from the various cationic lipids were rehydrated in a solution of 10 mM HEPES, 10 mM MES, 10 mM ammonium acetate and 130 mM NaCl at a pH range of 2.5 to 12. The pH of each formulation was reassessed to ensure that the pH had not significantly deviated from the original solution and 180 μL of each formulation was mixed with 20 μL of 10 μM TNS in distilled water (for a final TNS concentration of 1 μM). The solutions were mixed by pipetting and incubated at room temperature for 10 minutes, before being analysed for fluorescence intensity using a 321 nm excitation and 445 nm emission wavelengths.

### Biological Application of Triazine Lipids

Cell Culture of Bone Marrow-Derived Macrophages. Bone marrow was extracted from the femurs and tibias of 6-12 week old C57BL/6J female mice as described by Akbar et al. 20,21 and cultured for 7 days in media containing 20 ng mL⁻¹ murine MCSF (Biolegend) [RPMI 1640 (Life Technologies no. 21870), 10% fetal bovine serum (Gemini), 2.5 mM L-glutamine, 10 mM HEPES, 0.1 mM β2-mercaptoethanol (β2-ME), 100 U mL⁻¹ penicillin, 0.1 mg mL⁻¹ streptomycin]. After 7 days, the cells were transferred to tissue culture 96 well plates (Corning) at a density of 100,000 in 20 μL of medium and allowed to settle overnight for subsequent assays.

Lactate Dehydrogenase Release (LDH) Toxicity Assay. For determination of cytotoxicity, mature bone marrow-derived macrophages (BMDM) were treated with 20 μL of the lipids (concentrations denoted in figure legend) diluted in 20 mM HEPES buffer, with HEPES buffer as negative control and 10% Triton X-100 as positive control. After 24 hours, the 96 well plates were centrifuged at 200×g for 5 minutes to remove debris and 100 μL of media was transferred to an untreated flat-bottom 96 well plate. Next, 100 μL of LDH reaction reagent purchased from Cayman Chemical (Ann Arbor, MI) was added to each and allowed to sit for 30 minutes at 37° C. Absorbance at 490 and 680 nm were measured using a BioTek Synergy H1 plate reader and the data were processed using Microsoft Excel. Mean values from triplicates are shown for one of two independent experiments.

Gel Shift Assays Using Plasmid DNA. Nanoparticles consisting of a 1:1 molar ratio of cationic lipid/DOPE were rehydrated in a 20 mM HEPES solution at pH 4. The nanoparticles were mixed at equal volumes (5 μL) with plasmid DNA (5 μL) at the amine to phosphate (N:P) ratios indicated in the figure legends and incubated at room temperature for 10 minutes. For the triazine lipids the amine quantity per lipid was assumed to be 2 (one per headgroup), while DOTMA was considered to have 1 amine per lipid. After 10 minutes, 10 μL of the lipoplex was mixed with 2 μL of 6 loading dye (Boston BioProducts) and loaded onto a 1% agarose gel containing 0.5 μg mL⁻¹ of ethidium bromide and run at 100 mV for 60 minutes. The gels were visualized and photographed using a Bio-Rad ChemiDoc XR system using the manufacturer's software.

Transfection of Luciferase Plasmid and Cell Viability. HeLa cells cultured in EMEM (ATCC) supplemented with 10% fetal bovine serum were transferred to a 96 well plate, in quadruplicate, at a density of 20,000 cells per well and incubated for 24 hours at 37° C. in 5% CO2. Liposomes made with a 1:1 ratio of DOPE and TZ lipid were added to 200 ng pGL3 Luciferase Reporter Vector (Promega) at N:P ratios of 2.5, 5 and 10, and incubated at 37° C. for 10 minutes before being diluted in 100 μL of nonsupplemented EMEM and added to the cells. Following a four hour incubation at 37° C., the media was changed, and the cells were incubated for another twenty hours, at which point the cells were lysed with a cell culture lysis reagent at pH 7.8 composed of 25 mM Tris-phosphate buffer, 0.7 g L⁻¹ 1,2-diaminocyclohexane, 10% glycerol, 1% Triton X-100, and 1% protease inhibitor cocktail (Millipore). Total protein content was determined with a bicinchoninic acid assay (G-Biosciences) and luciferase protein expression was quantified by a luciferase assay (Promega). Cell viability was assessed using a Cell Titer Blue assay kit (Promega) based on the manufacturer's instructions. In each of the three independent experiments performed, transfection was compared with cells treated with Lipofectamine 3000 (Thermo), following the manufacturer's instructions, and with DNA treated cells.

Transfection of Plasmid Expressing Human Alpha-1 Antitrypsin (hAAT).

HEK293-T cells were seeded, in triplicate, on 24 well plates at a density of 50 000 cells per well using D-MEM containing 10% fetal bovine serum (Gemini), 100 U mL⁻¹ penicillin, 0.1 mg mL⁻¹ streptomycin and 500 μg mL⁻¹ geneticin (VWR) and incubated until they reached 70-90% confluency. Lipoplexes were formed by combining TZ lipid liposomes made with a 1:1 ratio of DOPE and TZ lipid in Opti-MEM (Thermo) with 400 ng of human alpha-1 antitrypsin (hAAT) plasmid DNA (Addgene no. 126704) and incubating for 12 minutes in Opti-MEM, before being added to cells. After 24 hours the media was removed for evaluation of viability and replaced with fresh media. The cells were then incubated for another 72 hours and then transferred to 1.5 mL microcentrifuge tubes and centrifuged at 400 rpm for 5 minutes. The media was removed and assessed for hAAT via ELISA and the cells were lysed using RIPA buffer (Thermo) for determination of total protein concentration (Thermo). In each of the three independent experiments performed, transfection was compared with cells treated with Lipofectamine 3000 (Thermo), following the manufacturer's instructions, and with DNA treated cells.

Quantification of hAAT Expression. For quantification of hAAT expression, 50 μL of goat anti-hAAT polyclonal antibody (R&D Systems no. AF1268-SP) were plated at a concentration of lug mL⁻¹ in carbonate buffer, pH 9.7, in a Greiner high binding 96 well plate and incubated overnight at 4° C. The plate was then washed with 200 μL of phosphate buffered saline with 0.1% Tween-20 (PBS-T) four times and blocked with 100 μL of PBS with 0.05% casein (Beantown Chemical, 124240; PBS-C) for 1 hour at 37° C. The plate was then washed again, and 100 mL of fresh media from cells were plated, in duplicate, along with a standard curve made by serially diluting purified hAAT (OriGene no. RG202082) in PBS-C from 50 ng mL⁻¹ to 0.048 ng mL and incubated for 1 hour at 37° C. The plate was then washed and 50 μL of mouse anti-hAAT monoclonal IgG2a antibody (R&D Systems no. MAB1268-SP) were plated at a concentration of 1 μg mL⁻¹ and incubated at 37° C. for 1 hour. The plate was washed again, and 100 mL of HRP conjugated goat anti-mouse IgG2a (Abcam no. 98698) was added at a 1:5000 dilution and incubated for 30 minutes at 37° C. The plate was then washed six times and binding was quantified by incubating the samples with 100 μL of tetramethylbenzidine (Rockland) for 30 minutes at room temperature, followed by quenching with 100 μL of 0.5 M H2SO4. Absorbance at 450 nm was recorded using a BioTek Synergy H1 microplate reader. After quantifying hAAT using the standard curve, hAAT in each well was normalized to total cell protein respective plate, which was quantified using a Pierce BCA Assay Kit (Thermo) using the manufacturer's instructions.

Mouse Immunizations. Liposomal immunizations were administered subcutaneously to three groups (n ¼ 5 per group) of eight-week-old female C57BL/6J mice (The Jackson Laboratory) housed in a specific pathogen-free facility at the University of Kentucky. The immunization, administered at 8 and 10 weeks of age, consisted of 50 μL of a 20 mM liposomal formulation prepared with a mixture of DMPC, DMPG, cholesterol, and monophosphoryl lipid A (MPL; Sigma) at a 15:2:3:0.3 molar ratio and 0.5 mg mL⁻¹ of lipid-conjugated peptide. The peptide used for these experiments was the lecithin-cholesterol acyltransferase domain of apolipoprotein A-I (sequence BAGGLSPVAEEFRDRMRTHVDSLRTQLAPHSEQMRESLAQRLAELKSN). As a control, the original peptide anchor (cholesterylhemisuccinate) was used to immunize one group of mice, while two other groups were immunized with the peptide was conjugated to intermediate D and the third group was immunized with peptide free liposomes. To assess the efficacy of immunizations, blood was collected by superficial temporal vein puncture using a small animal lancet (Medipoint) into a microcentrifuge tube and centrifuged for 10 min at 21 000×g after standing at room temperature for 2 h. Serum was stored at −80° C. for later antibody detection. The mice were sedated during any procedures using isoflurane gas. All animal procedures were performed in accordance with the United States Department of Health and Human Services, Office of Laboratory Animal Welfare, Public Health Service Policy on Humane Care and Use of Laboratory Animals and approved by the University of Kentucky Institutional Animal Care and Use Committee.

Apolipoprotein A-I Peptide Titer ELISA. Biotinylated apolipoprotein A-I peptide was diluted to a concentration 2 μg mL⁻¹ in phosphate buffered saline with 0.1% Tween-20 (PBS-T) and plated in a 96-well streptavidin-coated plate (Thermo Fisher No. 05124) using a volume of 100 μL. The peptide was incubated for 2 h at 37° C., then washed six times with 200 μL of PBS-T. Mouse serum (100 μL) was serially diluted in phosphate buffered saline containing 0.05% casein (PBS-C; Beantown Chemical) in duplicate, starting at 1:200 and incubated for 30 minutes at 37° C. The wells were then washed six times and treated with 100 μL of goat anti-mouse IgG-HRP (Invitrogen no. 16066) diluted 1:2000 in PBS-C and incubated for 30 minutes at 37° C. before being washed again. Binding was quantified by incubating the samples with 100 mL of tetramethylbenzidine (Rockland) for 30 minutes at room temperature, followed by quenching with 0.5 M H2SO4. Absorbance at 450 nm was recorded using a BioTek Synergy H1 microplate reader. Reciprocal endpoint titers were then calculated by plotting the absorbance vs. serum dilution and dividing the slope of the curve by two times the average of the blank (PBS-C only) wells.

Statistics. Statistical comparisons were performed using Graph Pad Prism version 7.

### Results and Discussion

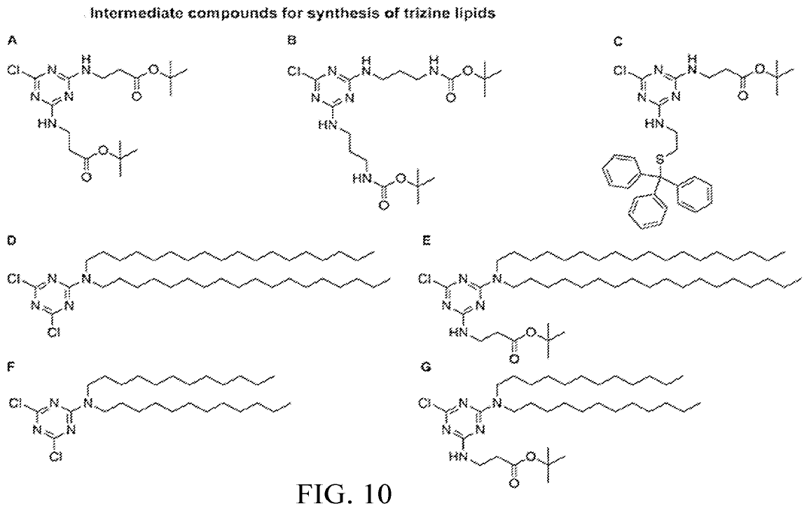

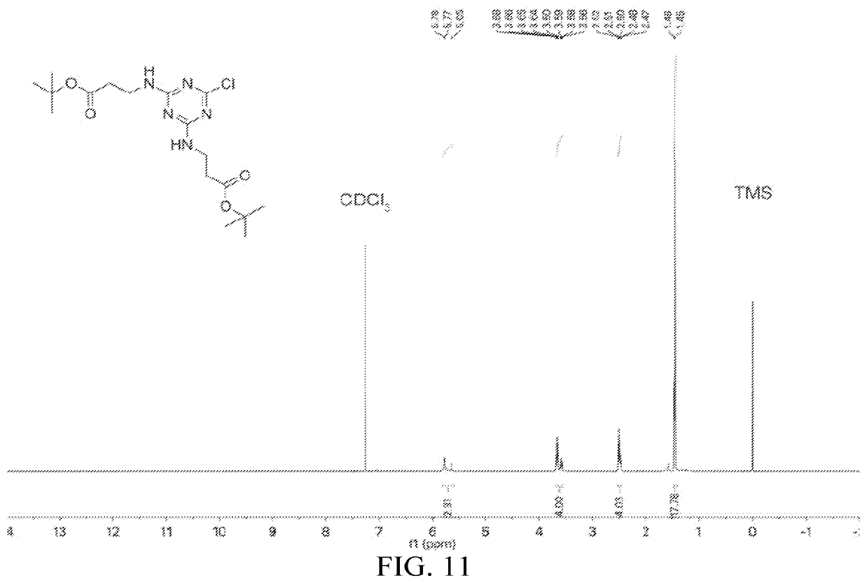

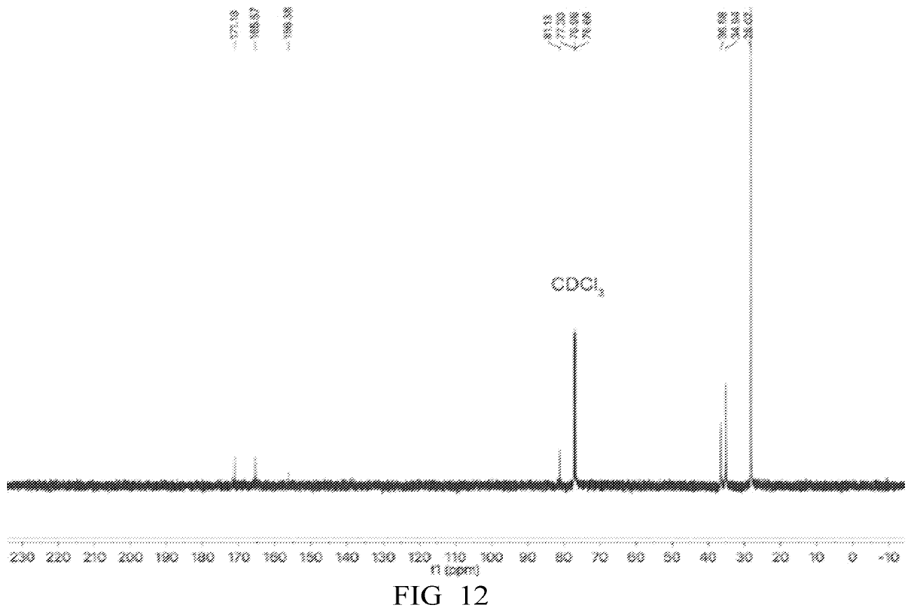

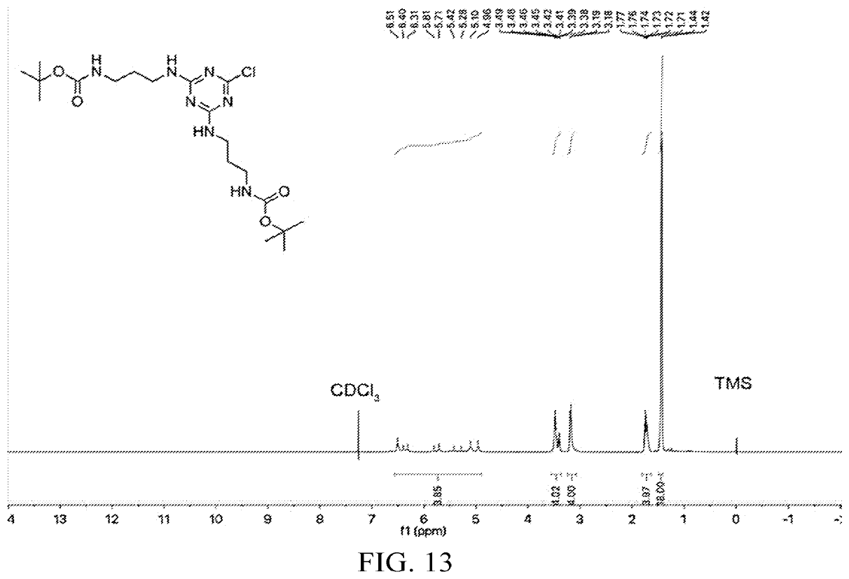

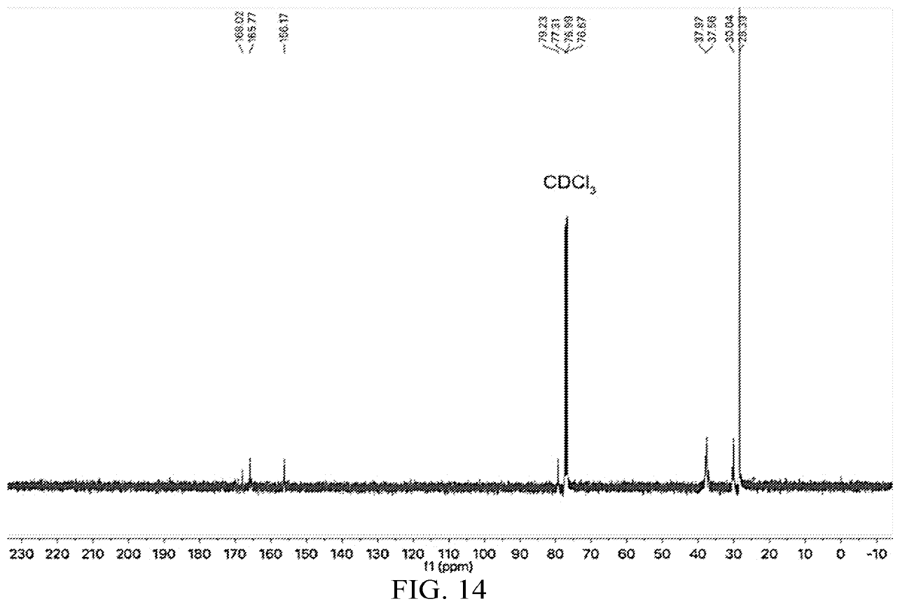

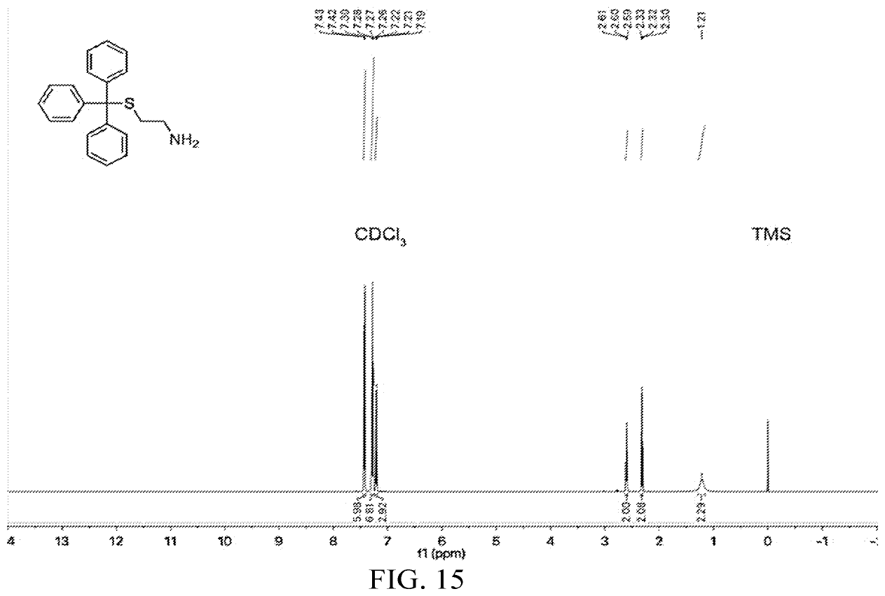

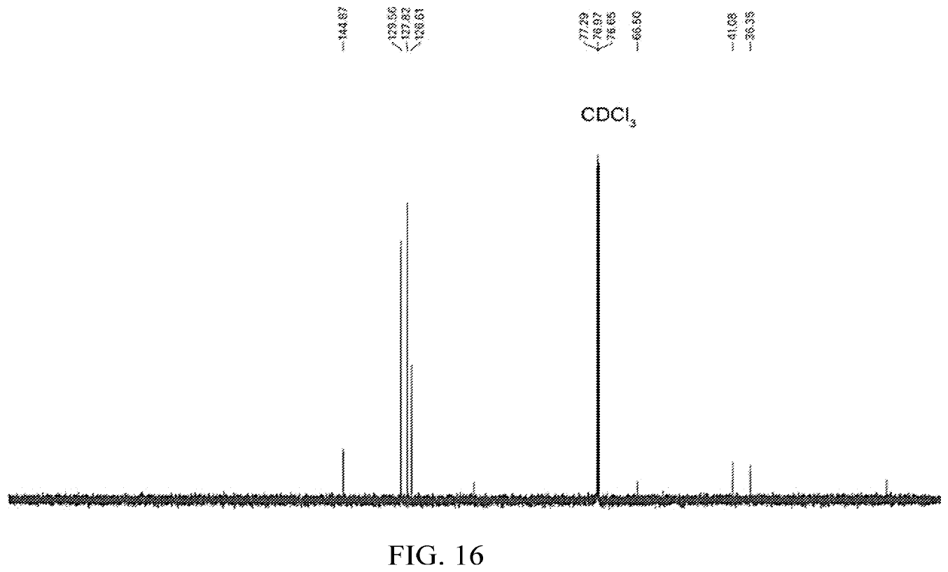

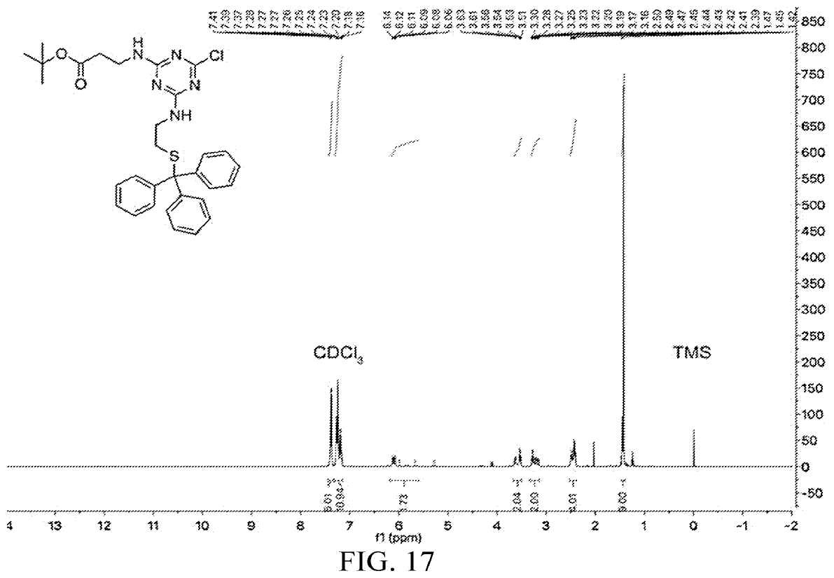

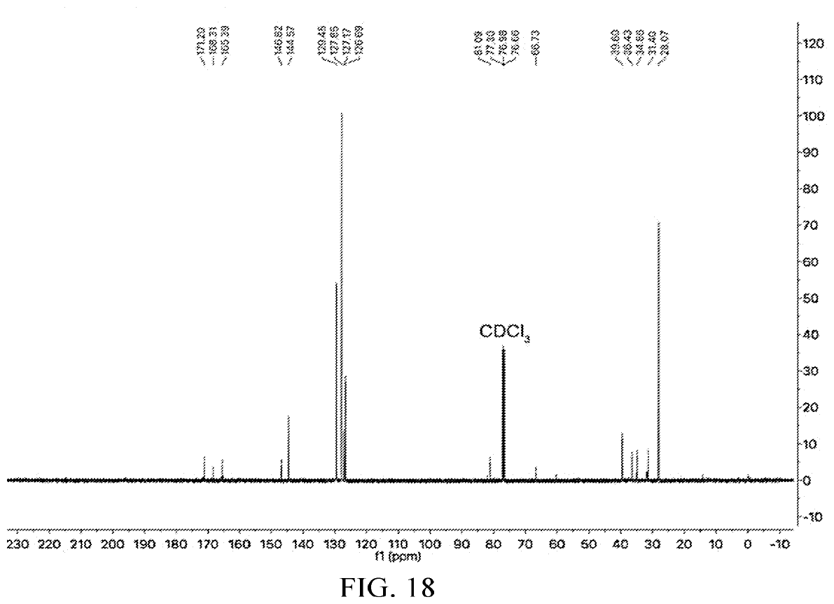

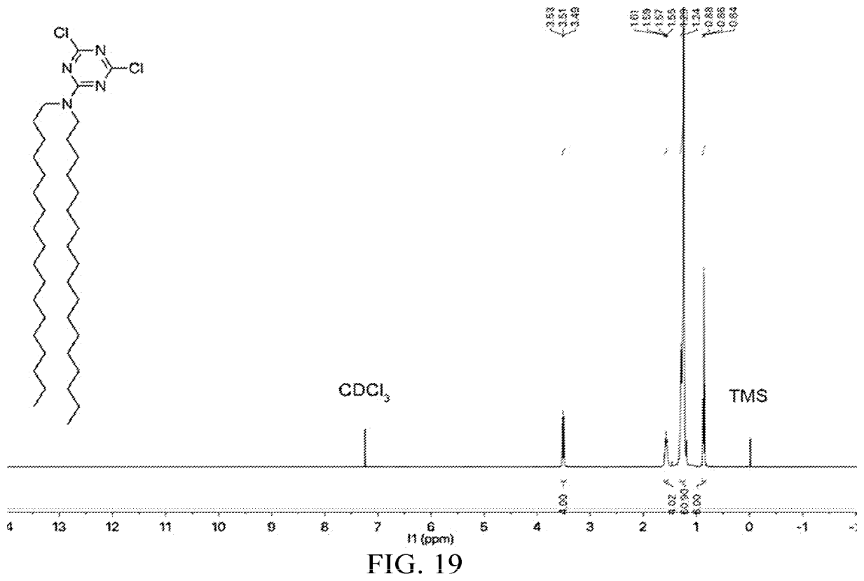

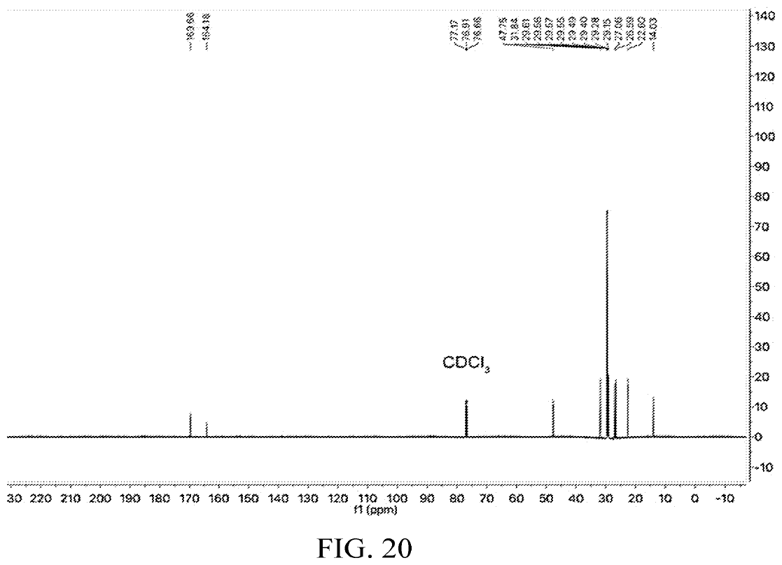

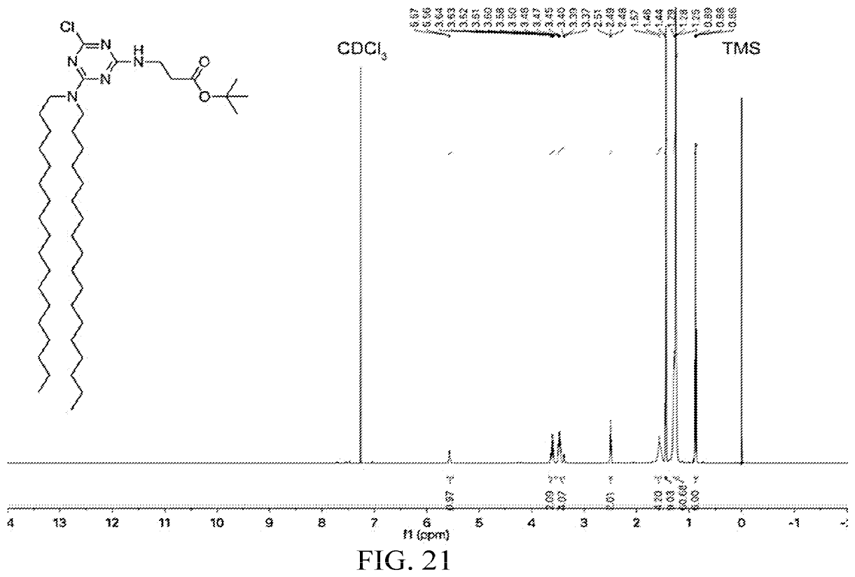

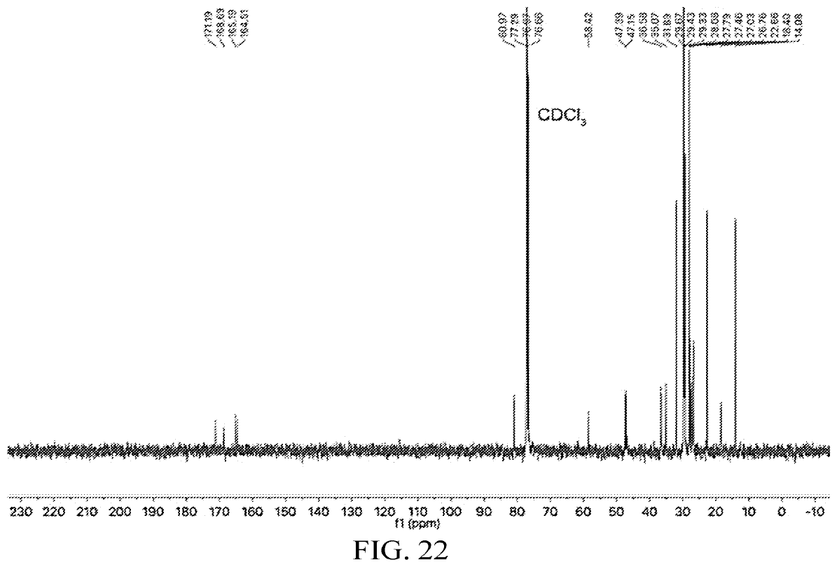

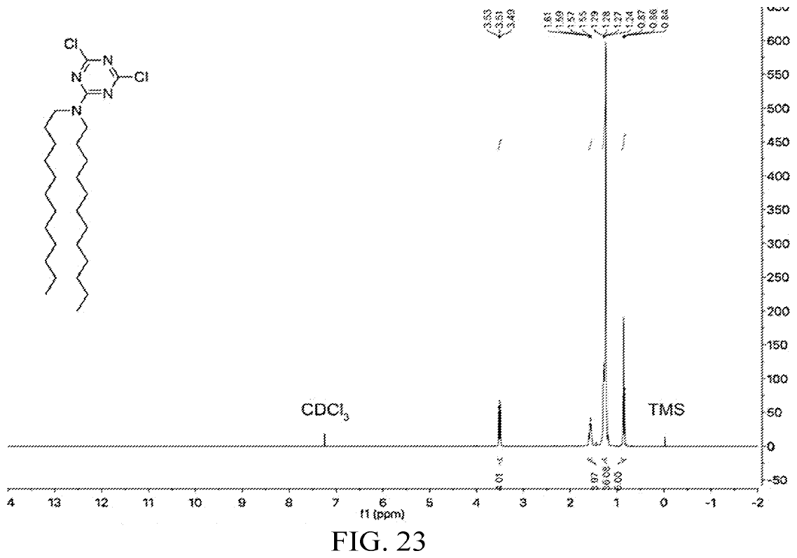

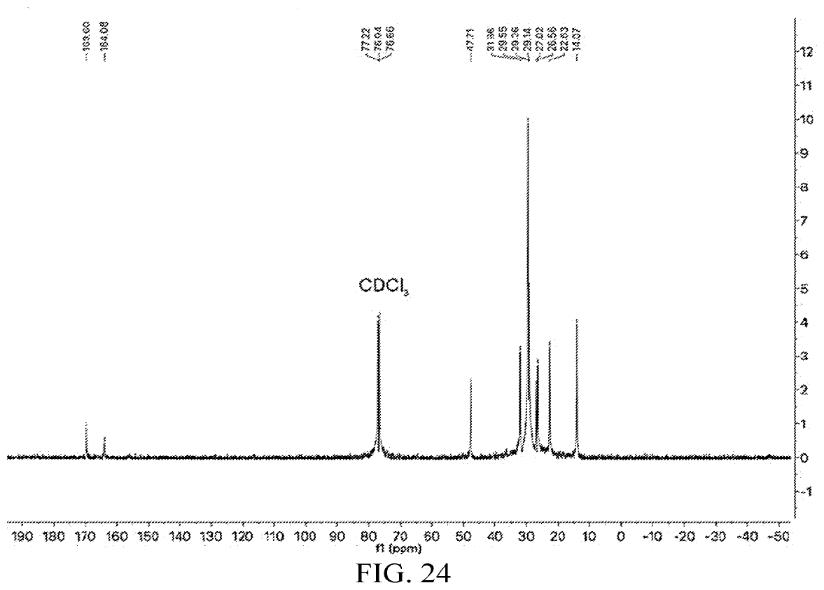

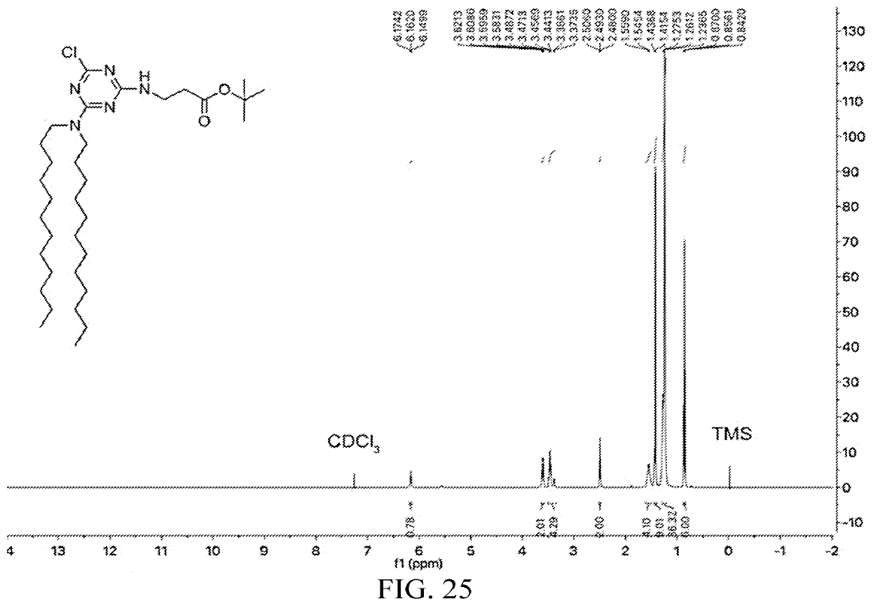

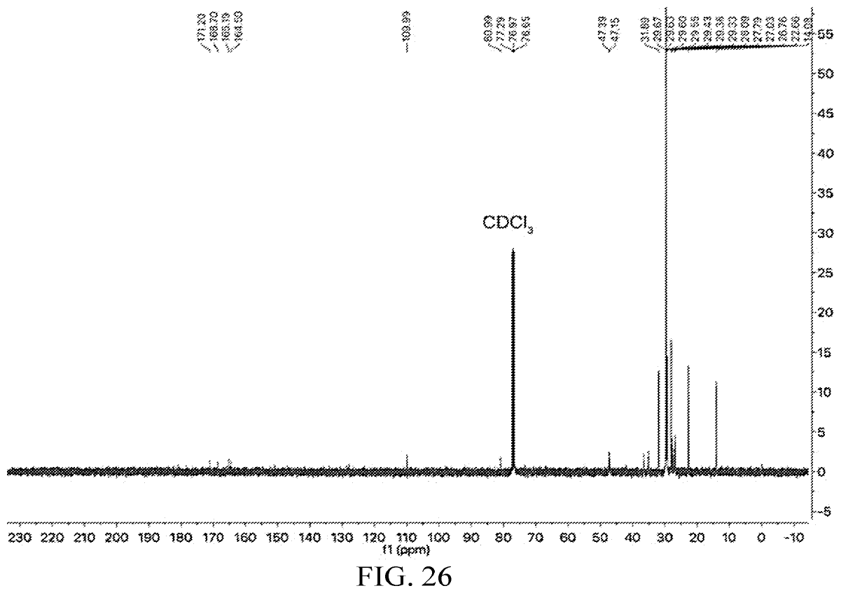

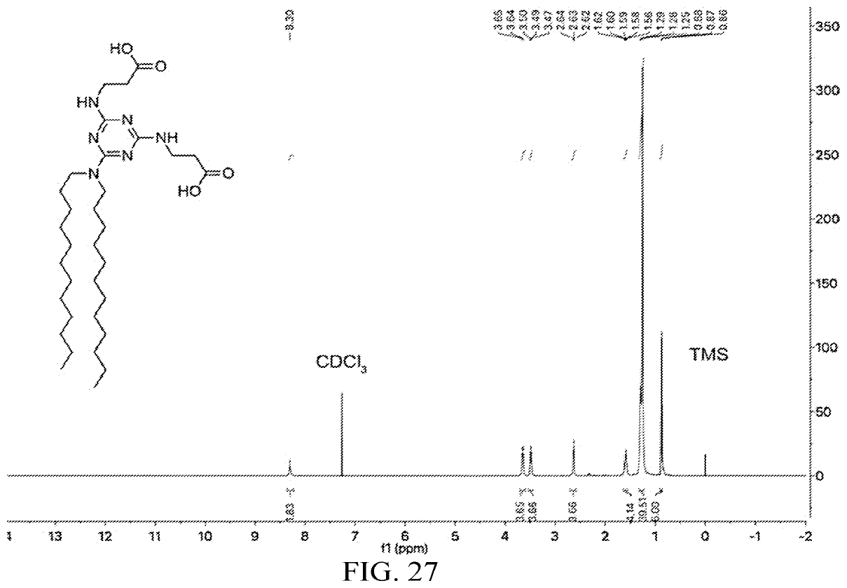

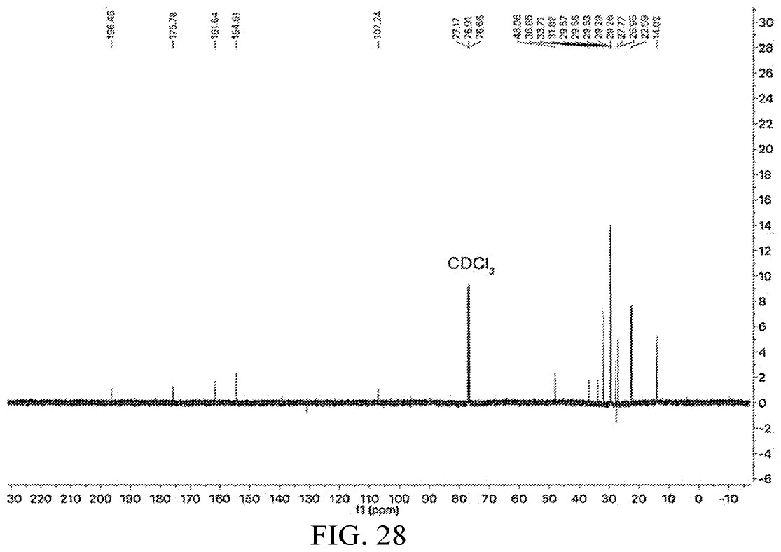

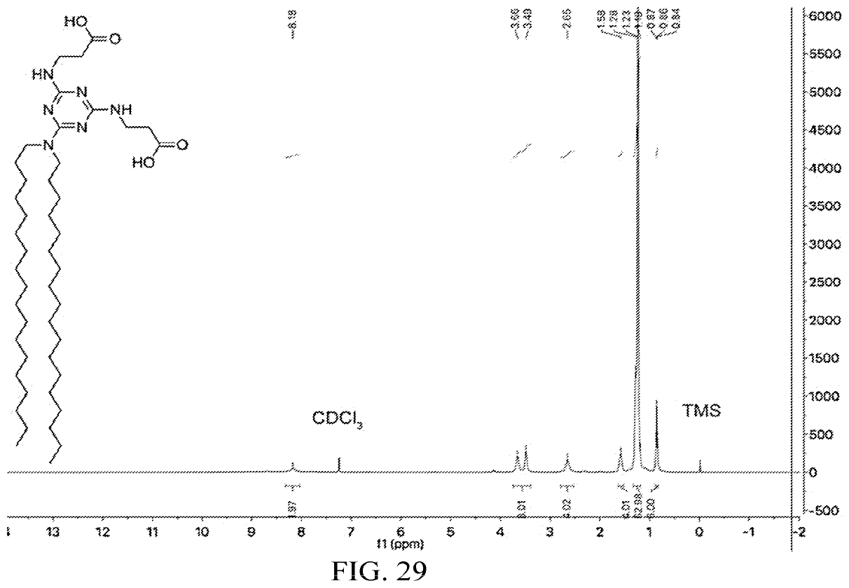

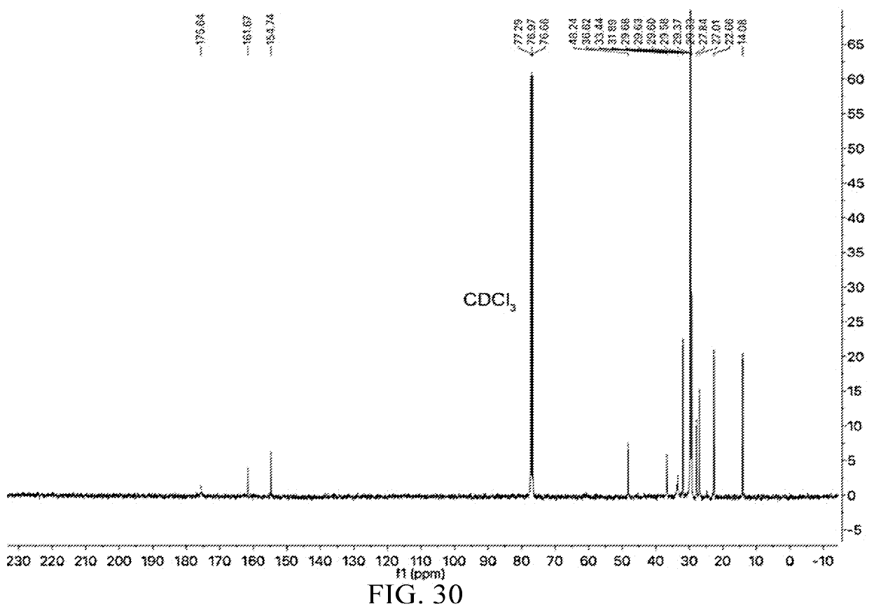

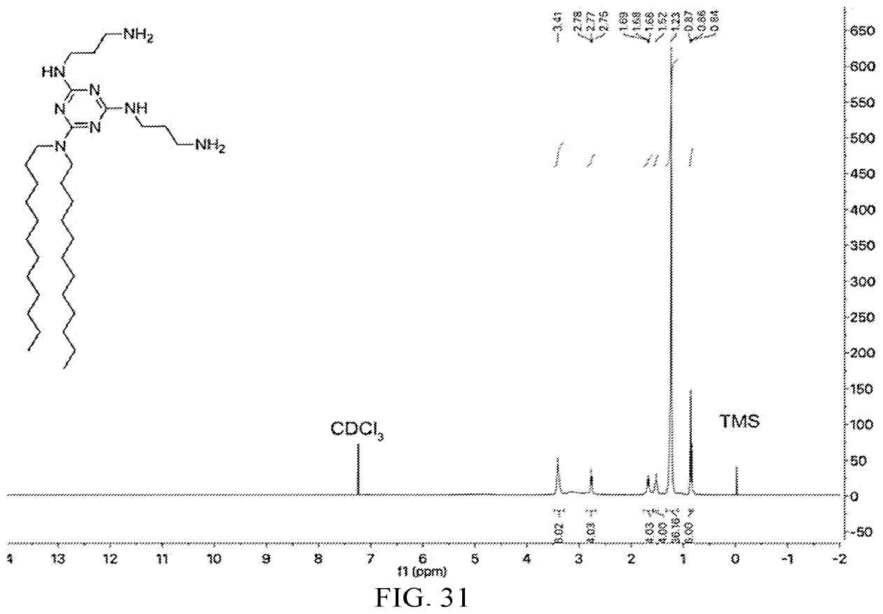

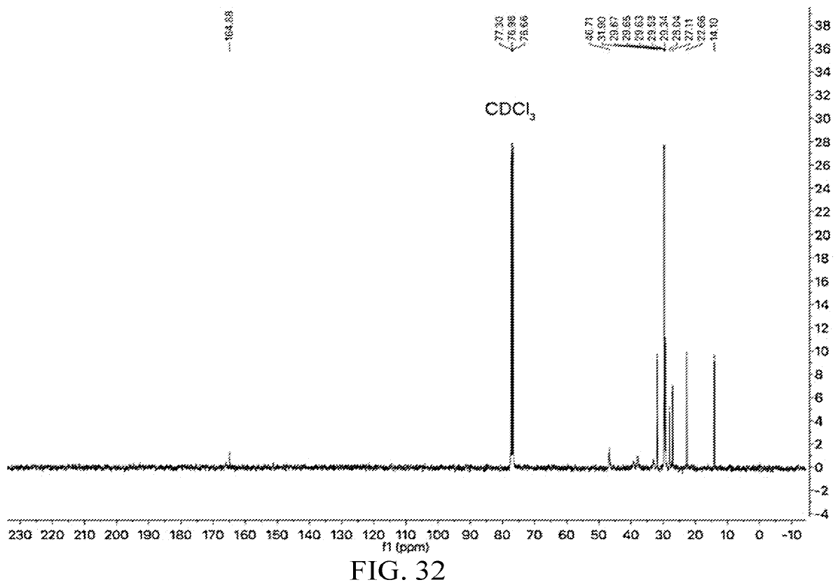

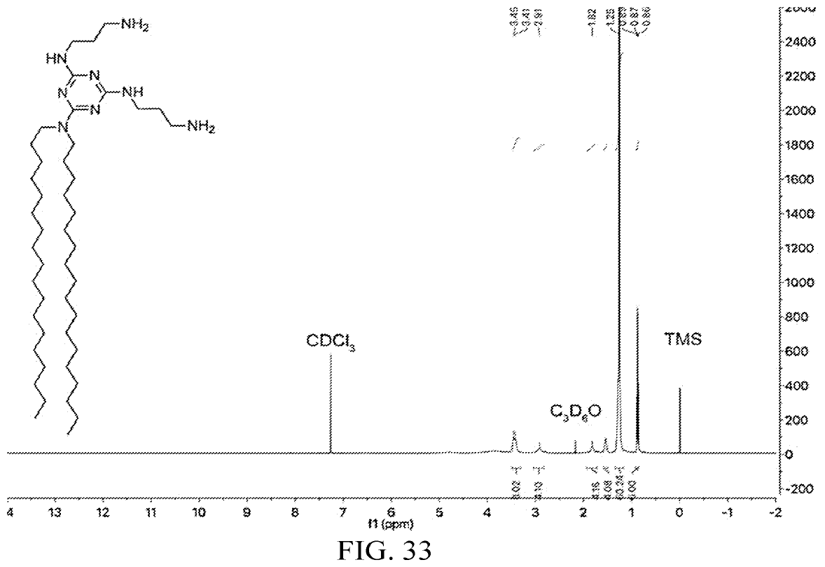

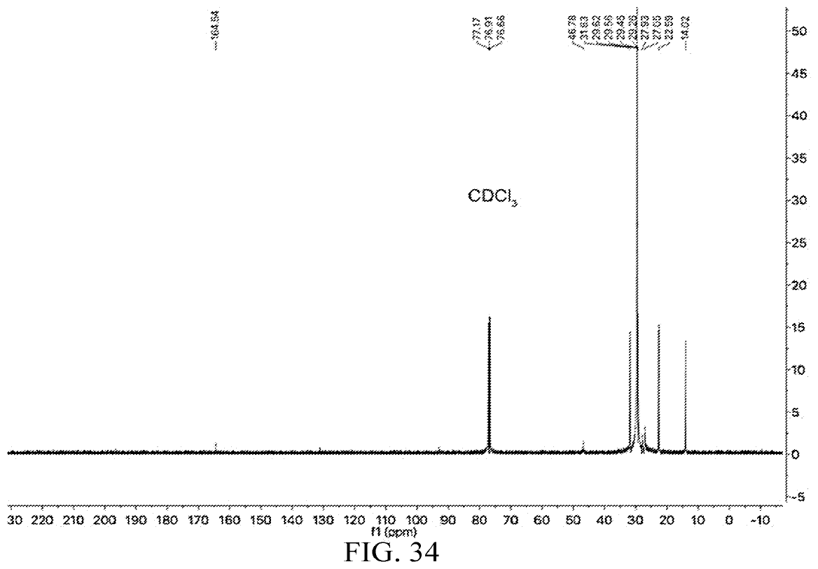

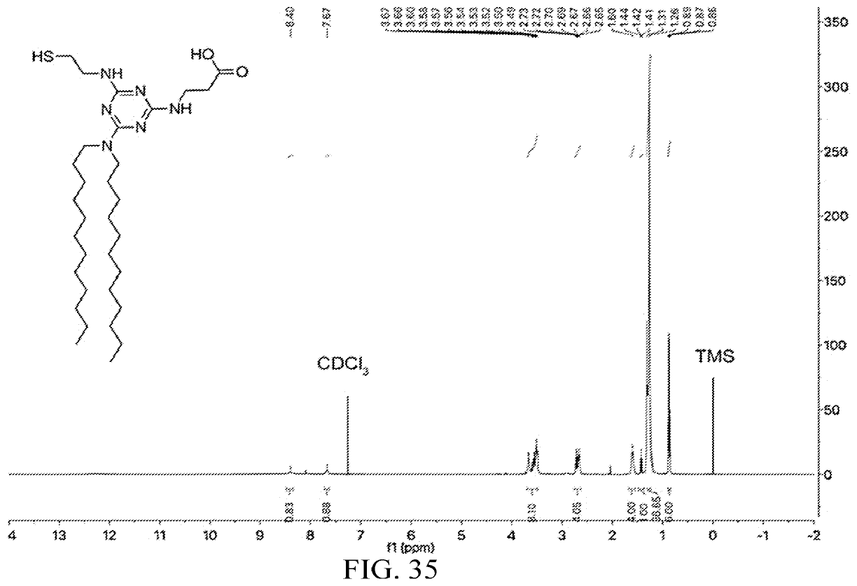

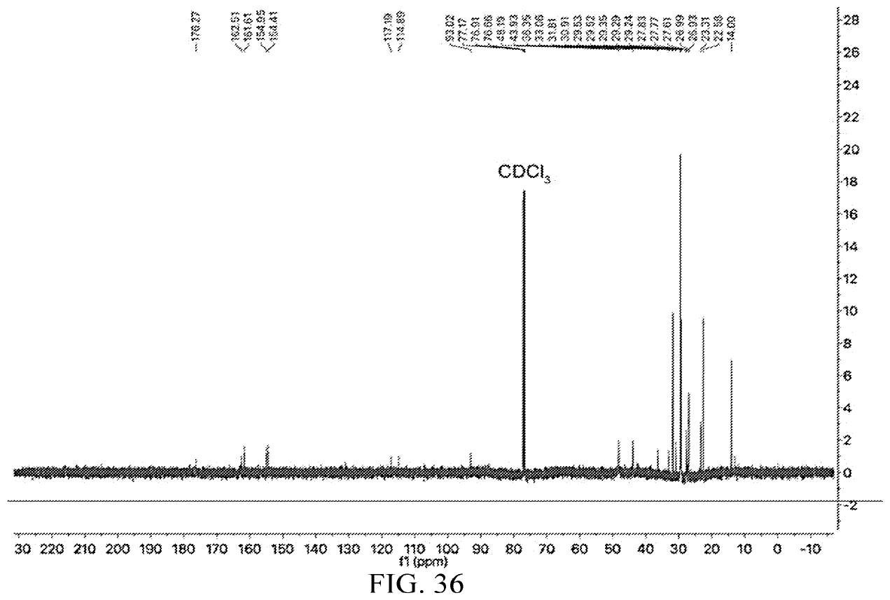

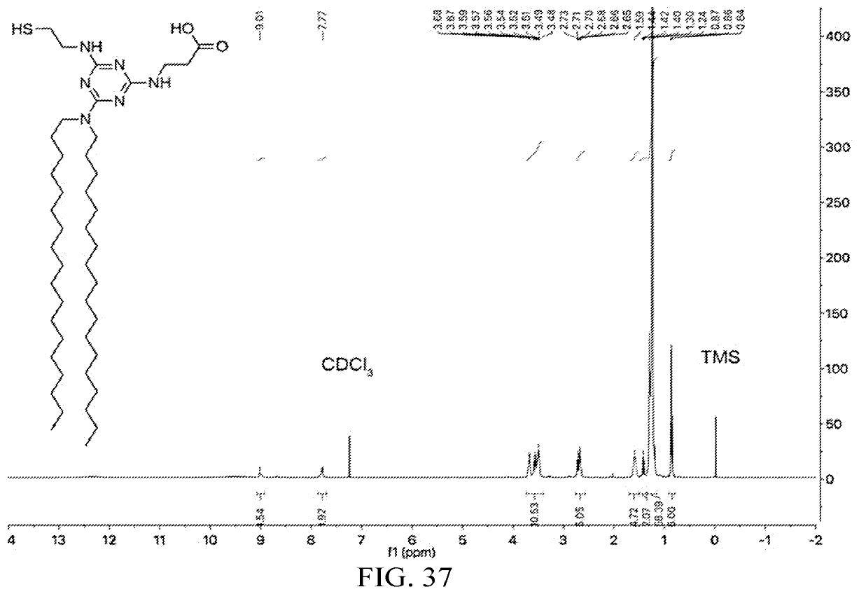

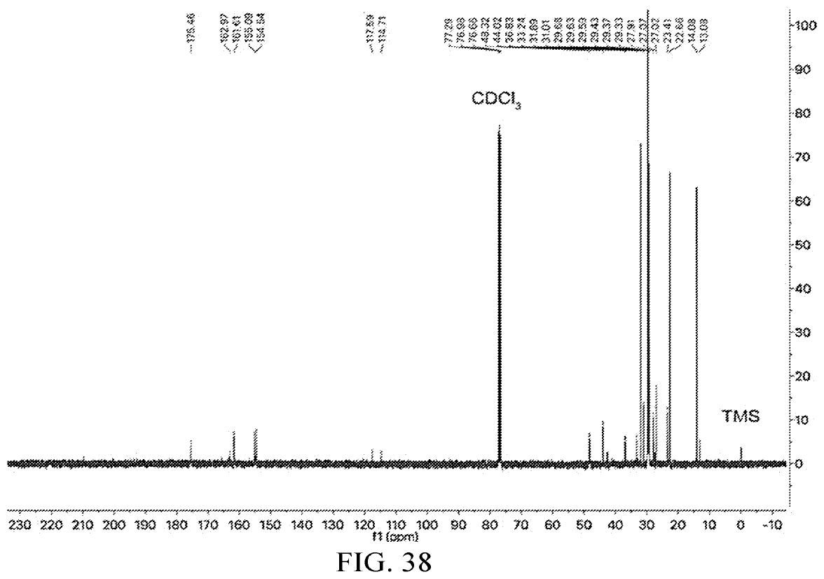

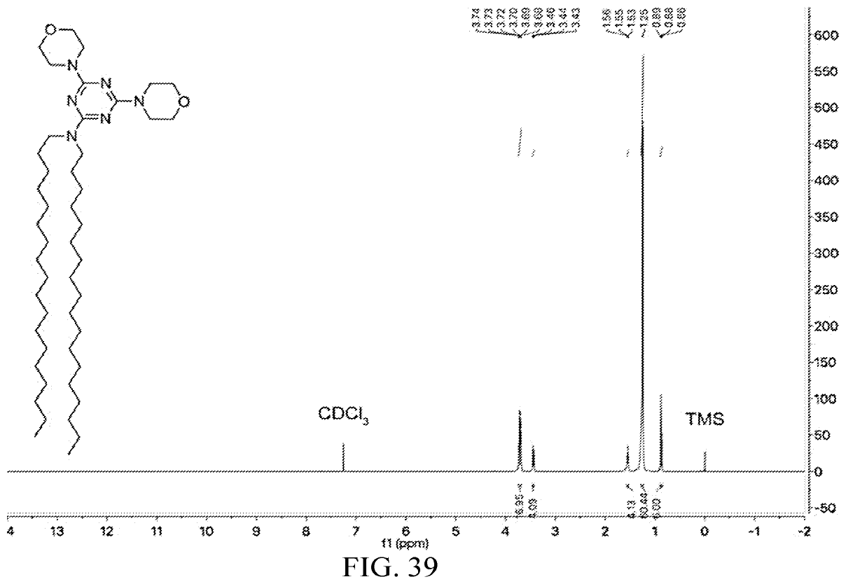

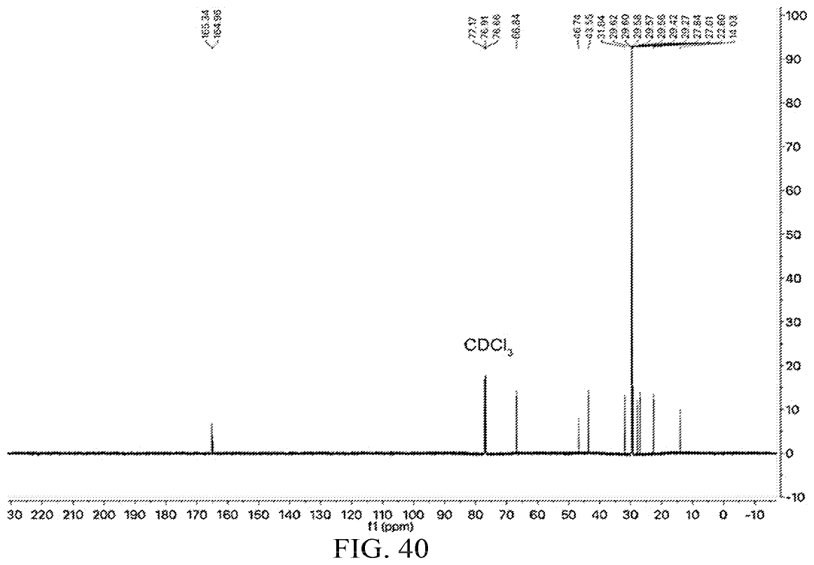

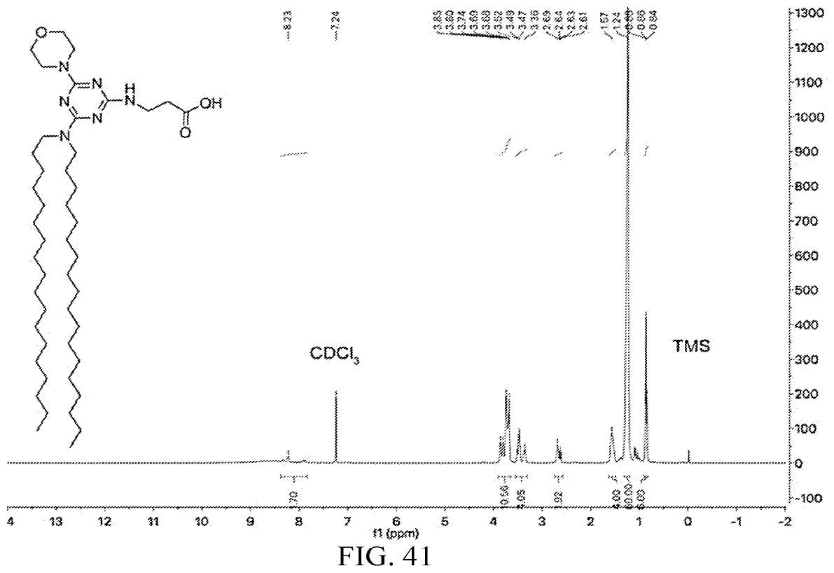

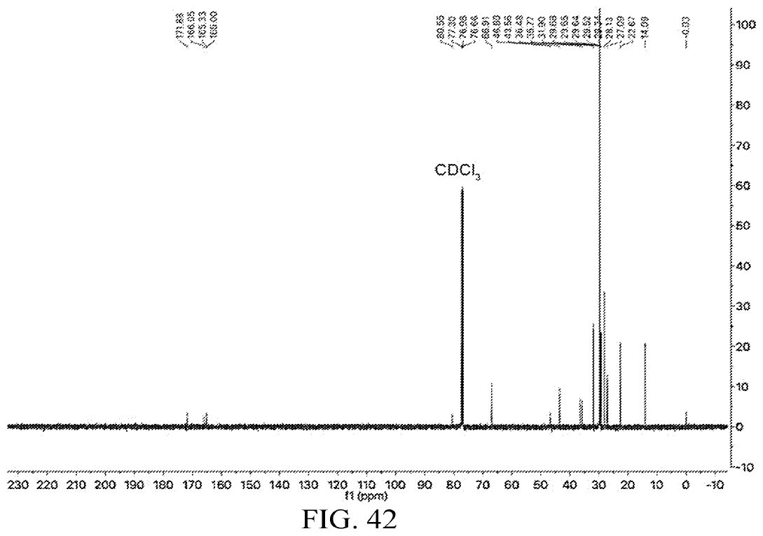

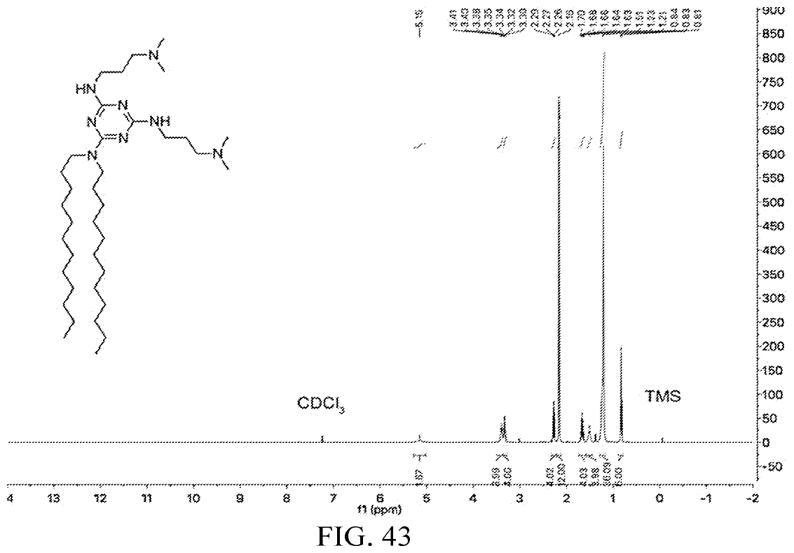

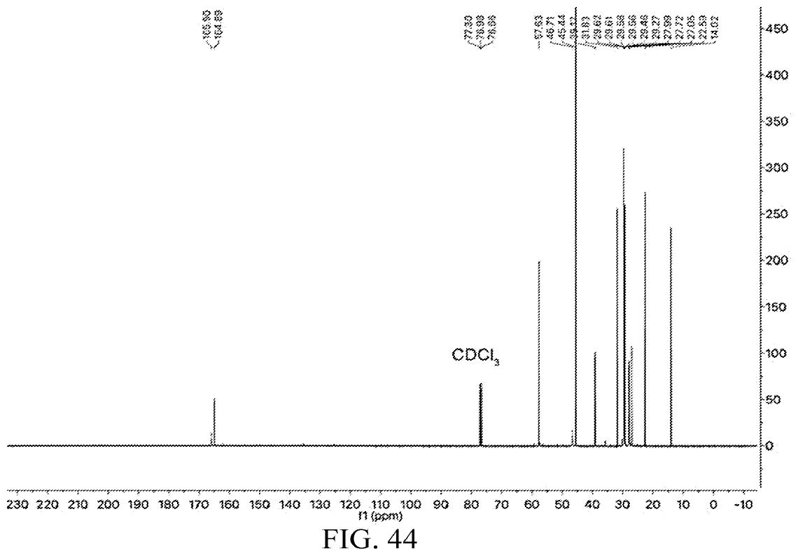

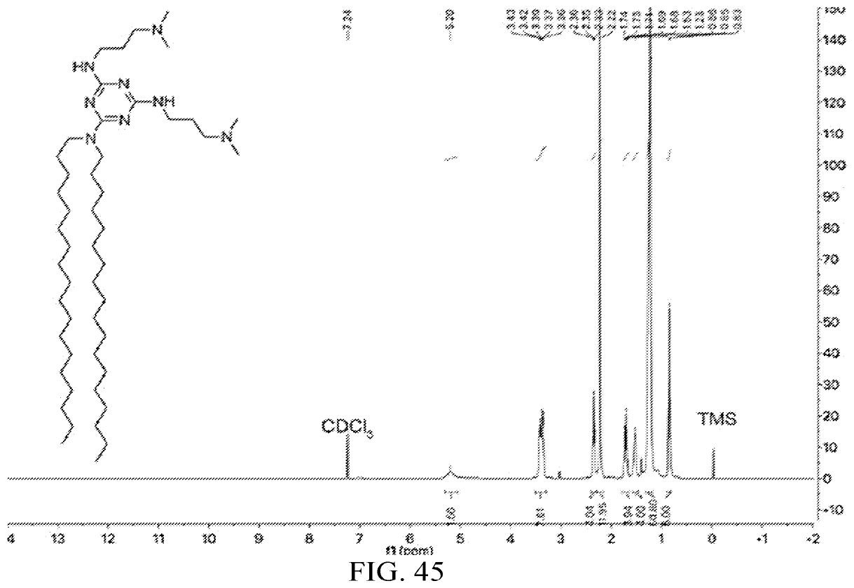

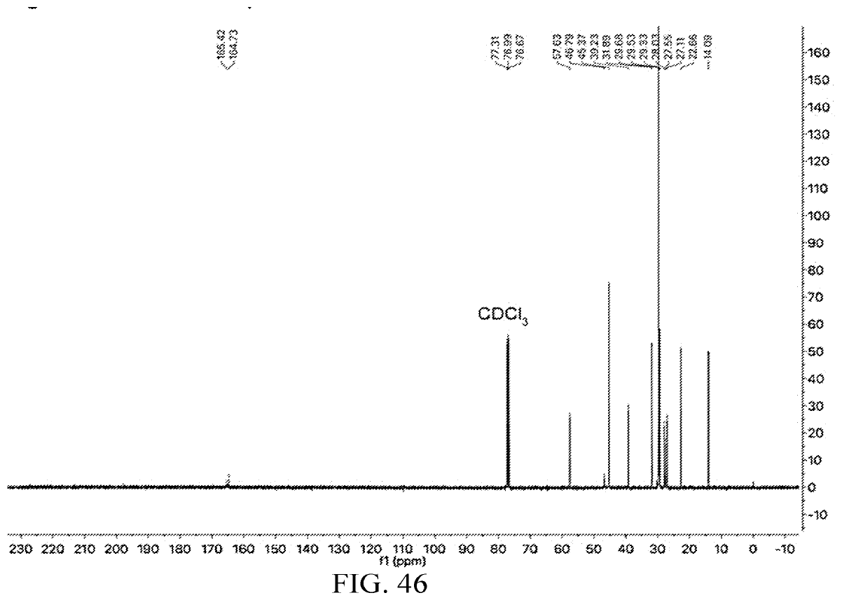

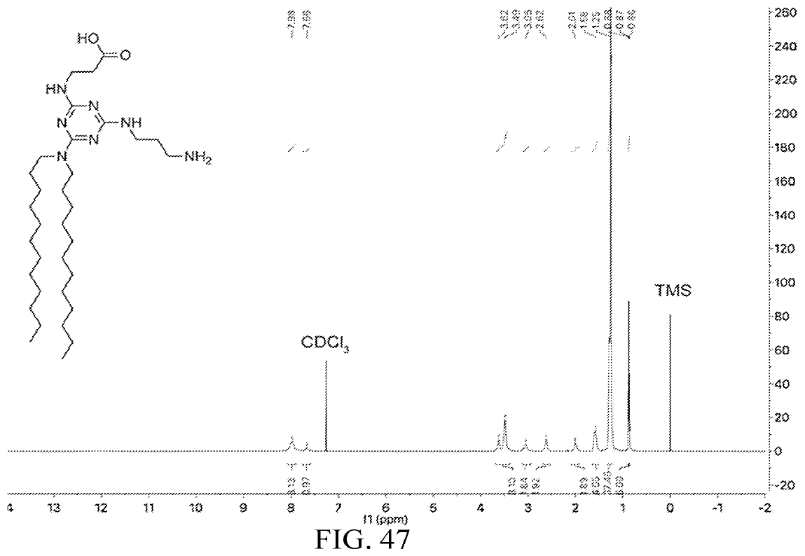

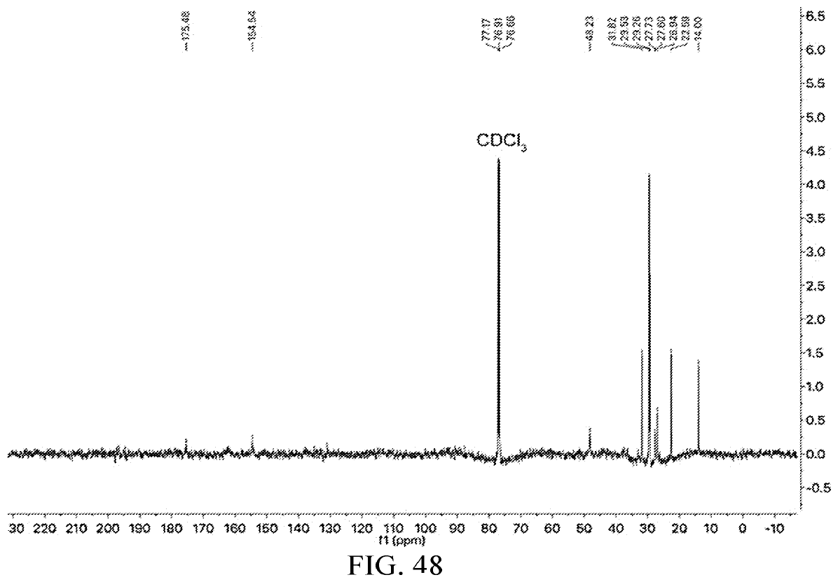

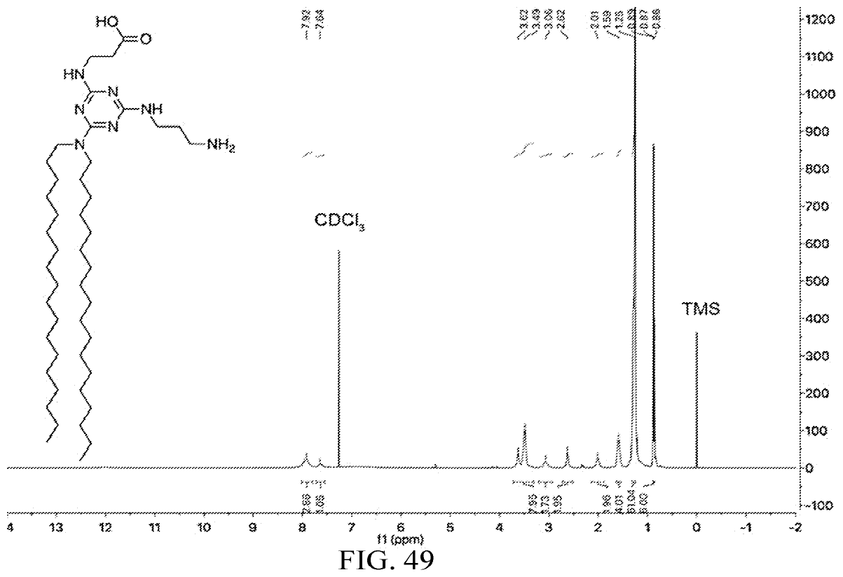

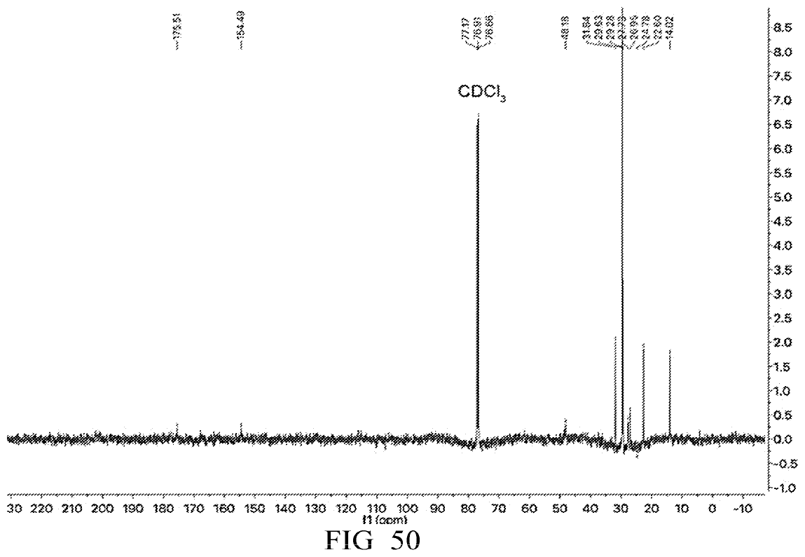

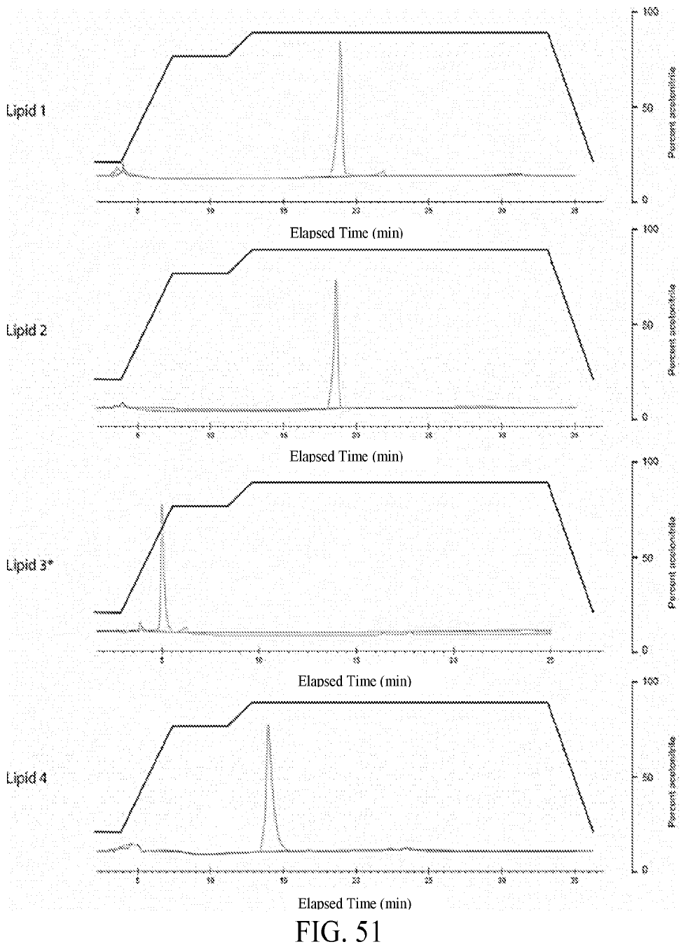

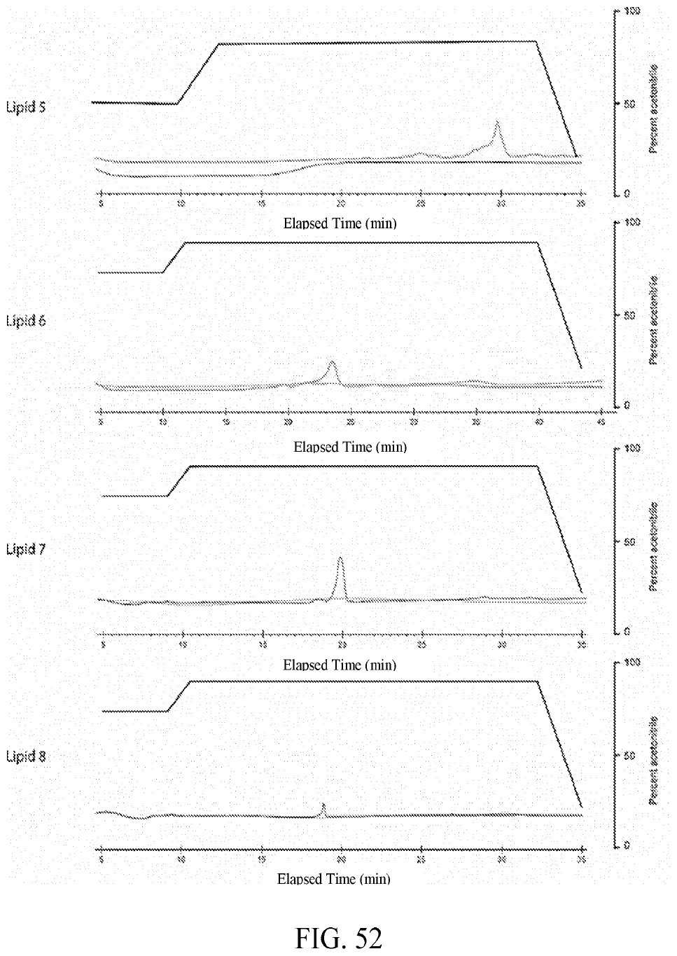

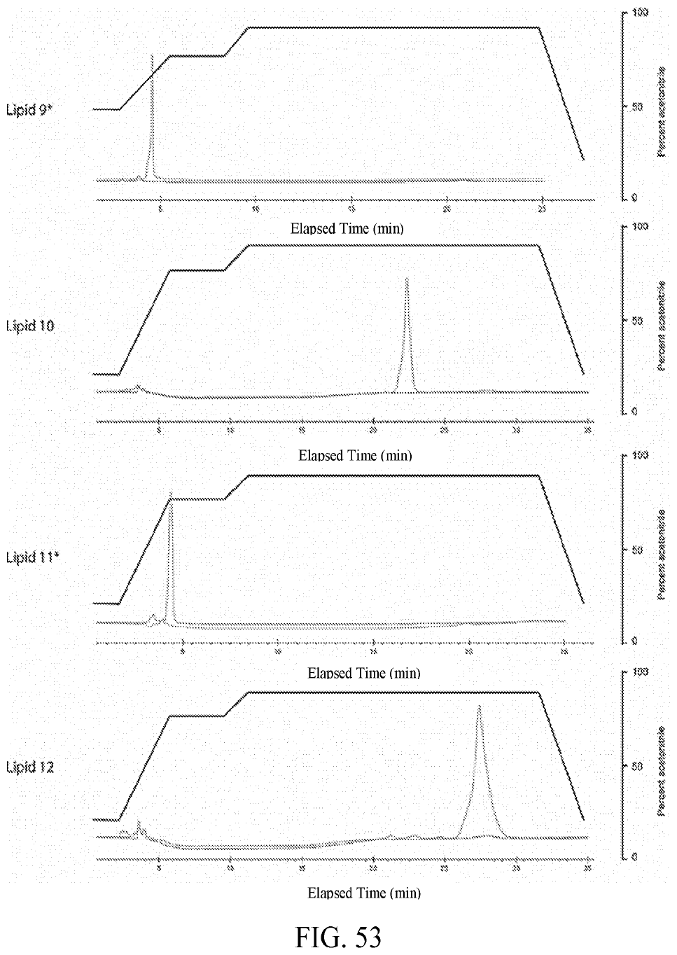

The thermally controlled, chemo-selective reactivity of cyanuric chloride provides a platform to add a multitude of functional headgroups and develop a wide array of synthetic lipids.¹⁴ In general, cyanuric chloride undergoes nucleophilic aromatic substitution at 0° C. for the first substitution, 25° C. for the second, and 70° C. for the third but the reactions are influenced by the nucleophilicity and steric hinderance of the reactants. Using this framework, two dichlorotriazine molecules were generated as the basis for lipids and several small molecules were tested as headgroups (FIGS. 1 and 10). The relative scarcity of commercially available long-chain secondary amines (tails) as compared to the abundance of potential head groups, results in a system in which compositional diversity is introduced in the headgroups rather than the tails. Therefore, a divergent approach was initially utilized for the synthesis of these compounds by adding the lipid tails to prepare a dichlorotriazine that was further diversified with various headgroups.

This strategy, however, was not viable for all headgroups used, particularly those with sterically hindered moieties. Therefore, a convergent strategy was attempted by initiating the synthesis with the addition of headgroups to the cyanuric chloride ring to form a monochlorotriazine to which tails were then added (FIG. 1).¹⁴

Synthesis of all lipids (excluding those containing morpholine) was attempted using both routes for comparison (Table 1) and the resulting products were characterized by NMR and HRMS.

TABLE 1
Structure, yield and characterization of triazine lipids^{a}

| | | | | | Tₘ | Size | | Charge | ID₅₀ |
|---|---|---|---|---|---|---|---|---|---|
| Lipid structures | # | Tail | Conv. | Div. | (° C.) | (nm) | PDI | (mV) | (μM) |
| 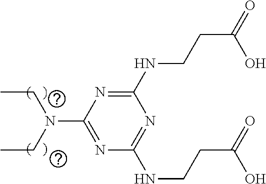 | 1 2 | C12 C18 | 22 20 | 49 21 | <10 58 | 124 ± 2 131 ± 0 | 0.24 ± 0.02 0.24 ± 0.00 | −63 ± 4 −59 ± 5 | 1098 894 |
| 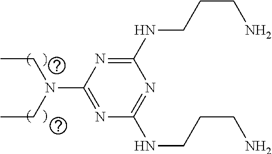 | 3 4 | C12 C18 | 28 48 | 43 53 | <10 33 | 130 ± 2 326 ± 1 | 0.41 ± 0.02 0.28 ± 0.00 | 52 ± 3 63 ± 3 | 111 180 |
| 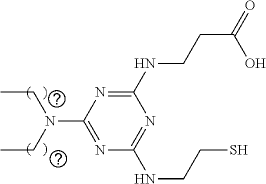 | 5 6 | C12 C18 | 90 72 | ND ND | <10 40 | 87 ± 2 93 ± 2 | 0.40 ± 0.00 0.27 ± 0.01 | −73 ± 6 −71 ± 5 | 679 988 |
| 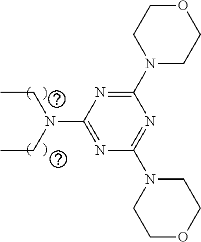 | 7 | C18 | ND | 91 | 61 | ND | ND | ND | ND |
| 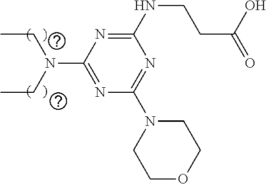 | 8 | C18 | ND | 80 | 51 | ND | ND | ND | ND |
| 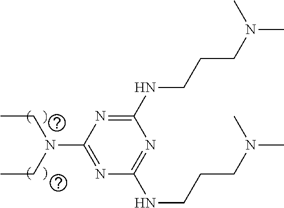 | 9 10 | C12 C18 | ND ND | 88 89 | <10 28 | 159 ± 1 186 ± 1 | 0.24 ± 0.00 0.28 ± 0.01 | 45 ± 2 47 ± 1 | 337 261 |
| 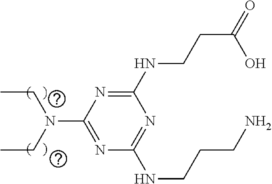 | 11 12 | C12 C18 | ND ND | 46 21 | <10 42 | Unstable Unstable | Unstable Unstable | 42 ± 5 70 ± 5 | 277 227 |

^{a}Yield (%) is calculated based on all synthetic steps, including first divergent step to prepare dichlorotriazine intermediates D and F, which are used for all subsequent headgroup reactions.
ND = Not determined.

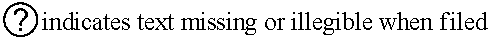

Lipids 1˜4 proceeded well under both routes with similar yields for the convergent and divergent route using the beta-alanine headgroups (lipid 2: 20% and 21%) and the diaminopropane headgroups (lipid 4: 48% and 53%). This was not the case for lipids 5 and 6, which employed trityl-protected cysteamine (Trt-Cys). Divergent synthesis of lipidated dichlorotriazine molecules with Trt-Cys resulted in an insoluble compound with exceedingly low yield and could only be successfully synthesized using the convergent route with protected beta-alanine as the first substitution on cyanuric chloride.

Since the third addition to the TZ ring is generally more difficult to achieve, and morpholine is a strong nucleophile, 14 lipids 7 and 8 were only synthesized using the divergent route with overall yields of 91% and 80%, respectively. The divergent route was also used to synthesize lipids 9 and 10 containing N,N-dimethyldiaminopropane, as well as lipids 11 and 12 using both beta-alanine and diaminopropane in the headgroup. When using the convergent approach to synthesize these lipids, the purification of the monochlorotriazine headgroup molecules in the absence of the lipid tails was problematic requiring a slow and lengthy purification by column chromatography (>12 hours). Conversely, the divergent route facilitated synthesis and purification of the final lipids.

Using these two routes, the divergent synthetic route reduces the total number of reactions needed to prepare a library of molecules by 25-33% depending on the final composition of the lipids. While some lipids result in similar overall yield between convergent and divergent routes, the challenges with nucleophilicity, steric hinderance and purification of intermediate molecules resulted in synthetic preference for one route over the other for certain headgroups. The divergent route results in increased compositional diversity with fewer steps, while the convergent route serves as an important complementary role for the synthesis of certain lipids.

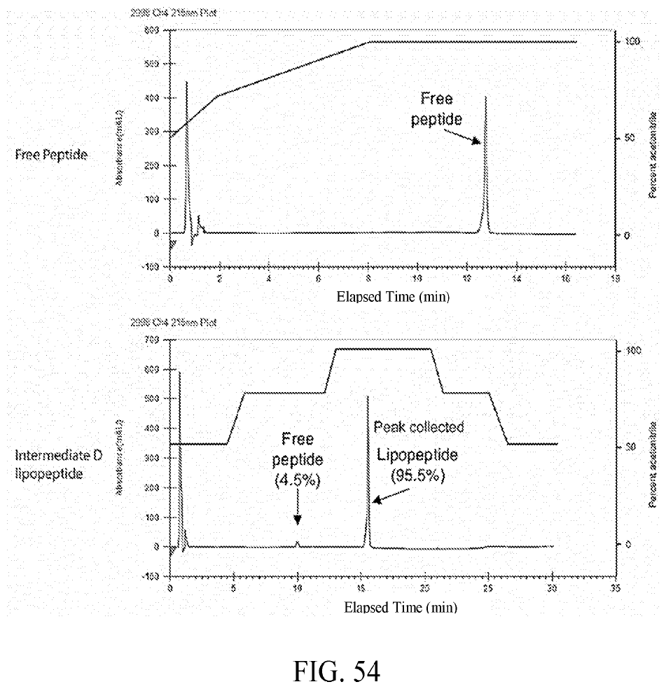

The utility of the divergent route was then explored further by reacting the C18 dichloro-triazine compound (intermediate D) with the N-terminal amine of a protected peptide on rink amide resin, to provide an alternative synthetic route to present lipid-anchored peptides in a liposomal bilayer for vaccination. Using the 44 amino acid sequence from apolipoprotein A-I (ApoA-I) previously investigated,²² 95% lipopeptide purity was achieved, which is improved as compared to previously described lipopeptide synthetic strategies using cholesteryl hemisuccinate and other lipid anchors.²³ In addition to the improved overall yield, the ease of lipopeptide synthesis using intermediate D provides a convenient platform for continued vaccination studies with minimal downstream purification, as evidenced by the HPLC trace of the lipopeptide (FIG. 54).

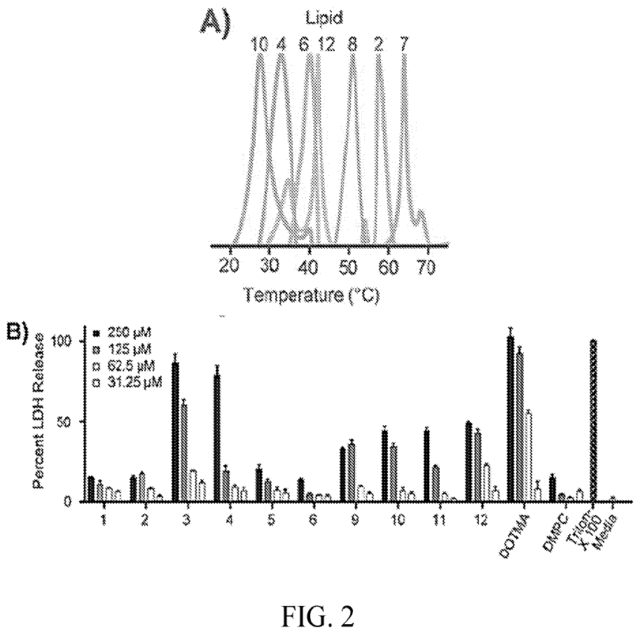

Next, liposomal formulations using each lipid were prepared. First, the transition temperature (Tm) of each compound was determined by forming nanoparticles of pure lipid via rehydration of thin lipid films in 20 mM HEPES and sonicated at high temperature. The resultant formulations were then transferred into Hastelloy ampules to assess the transition temperature by differential scanning calorimetry. All lipids made with a didodecylamine tail yielded a Tm below 10° C., while those containing a dioctadecylamine tail ranged in Tm from 28-64° C. (FIG. 2A and Table 1).

All lipids were initially formulated at pH 7, but failed to properly hydrate. Therefore, hydration of lipids 1 and 2 were tested at increasing pH and found that pH 10 was ideal for hydration. All other lipids hydrated well under acidic conditions (pH 4). Lipids 7 and 8, which contained morpholine in the headgroup, failed to form liposomes, alone or in combination with distearoyl phosphatidylcholine (DSPC) or DSPC and cholesterol from 5 to 90 mol % TZ lipids. Other lipids made with isonipecotic acid also failed to form liposomes (data not shown) indicating that steric hinderance of the headgroups may preclude liposome formation. Additionally, while lipids 11 and 12 initially formed nanoparticles, the structures were unstable past 24 hours as determined by dynamic light scattering. Aside from these, all other lipids formed nanoparticles that appear stable at one month after preparation when stored in the refrigerator, based on dynamic light scattering.

For lipids that formed nanoparticles, the size of the nanoparticles ranged from 87 to 186 nm in diameter (Table 1), with no clear trend between diameter and structural characteristics, such as lipid tail and charge. Lipids containing cysteamine in the headgroup achieved the smallest size, while lipids 9 and 10, exhibited the largest initial diameter. Lipids 11 and 12 also formed nanoparticles >300 nm in diameter, but are unstable as denoted in Table 1 with PDI of 0.98 and 0.51, respectively. The charges of each formulation also aligned with the headgroup used and ranged from −75 to 70 mV for anionic and cationic headgroups, respectively. Lipids 11 and 12, which contained beta-alanine and 1,3-diaminopropane in the headgroup, were hydrated in acidic conditions yielding a positive charge for this formulation. Attempts were made to formulate these in both acidic (pH 4) and basic (pH 10) solutions, but the lipids only hydrated well in acidic conditions.

Prior to testing the nanoparticles in further applications, the toxicity of the compounds was assessed in vitro. The primary mechanisms of toxicity associated with lipid nanoparticles, particularly cationic ones, are cell lysis and activation of immune responses. 24,25 Therefore, macrophages were chosen to test this aspect of TZ nanoparticles as these are among the primary cells responsible for the uptake of nanoparticles from circulation and are associated with the immune responses observed following in vivo nanoparticle administration.²⁶ To assess the toxicity of the nanoparticles in vitro, the lipids were tested for induction of lactate dehydrogenase (LDH) release from bone marrow derived macrophages (BMDMs) from C57BL/6J mice, which has been demonstrated to be a more sensitive method of early liposomal toxicity.²⁵ BMDMs were treated with TZ lipids at concentrations ranging from 31.25 to 250 nmol mL⁻¹. As can be seen in FIG. 2B and Table 1, the toxicity of the nanoparticles ranged between that of the synthetic, cationic lipid DOTMA, and the natural zwitterionic phospholipid DMPC (Table 1). The LD50 values of the cationic lipids were considerably higher than that of other lipids (133 and 180 mM for lipids 3 and 4, respectively), approximating the toxicity of DOTMA (LD₅₀=78 mM). Lipids 9 and 10 also had a higher toxicity than other TZ lipids (LD₅₀=337 and 261 mM, respectively), which did not differ significantly from DMPC (LD₅₀=969 mM).

The first assessment of the utility of the nanoparticles was performed by testing whether those TZ lipid nanoparticles that remained stable for several weeks could retain therapeutics in their aqueous core, and carboxyfluorescein (CF) encapsulation was used to test this.¹⁸ Intriguingly, when pure TZ lipids were used to encapsulate CF, they formed a gel indicating that they may be unable to encapsulate aromatic molecules even when mixed with DSPC at ratios as low as 10% TZ lipid.

Having shown great success in preclinical studies, many synthetic lipids with cationic headgroups are used in gene transfection as commercial reagents for laboratory use.³ More recently, the first siRNA therapeutic, partisiran, was approved for clinical use by the United States FDA and two-lipid-based mRNA vaccines were approved under emergency use for prevention of COVID-19.^{27,28}

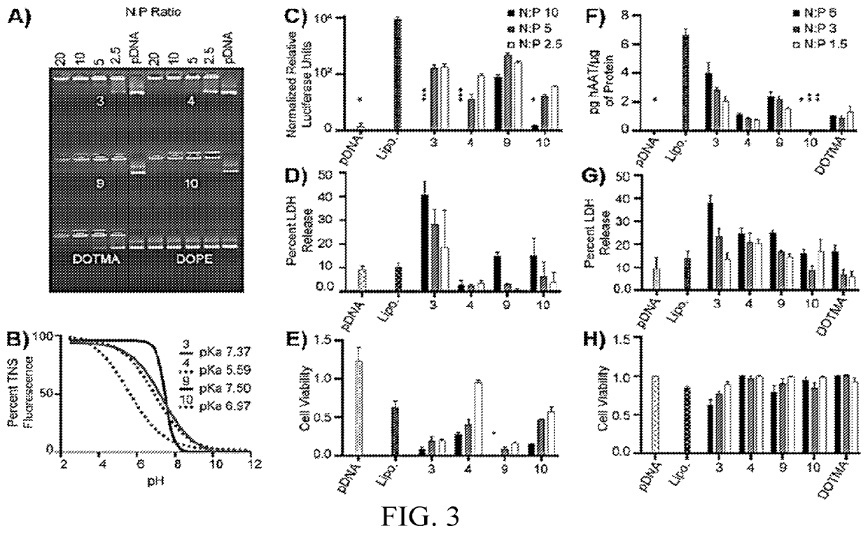

To determine whether TZ lipids with cationic headgroups that form stable formulations (3, 4, 9 and 10) could complex nucleic acids, lipid nanoparticles made from a 1:1 molar ratio of cationic lipids and DOPE were incubated with plasmid DNA at increasing ratios of cationic amine (N) to anionic nucleic acid phosphate (P) and assessed for migration in an agarose gel. Formulations with a 1:1 molar ratio of DOPE and cationic lipid have been extensively reported in the literature and provide a simple starting point for assessing the potential of cationic lipid formulations. 29,30 Of note, the N content of TZ lipids are based on the distal aliphatic amines of the headgroups, but the other amines in the molecules may contribute to complexation. As shown in FIG. 3A, all four lipids were able to complex RNA at an N:P ratio of 5 or above. By comparison, DOTMA/DOPE nanoparticles inhibited RNA migration at an N:P ratio of 10 while pure DOPE lipids were unable to prevent migration. The TZ/DOPE lipoplexes were also evaluated by DLS at N:P ratios of 1 and 5 and their size and charge was determined by DLS. As evidenced in Table 2, the lipoplexes increased in size, compared with the free nanoparticles, but they increased slightly in size as more DNA was added, suggesting complexation.

TABLE 2
Characteristics of liposomes made with DMPC, DOTMA, and various
lipid combinations, as well as immunization liposomes.

| Lipid | Size (nm) | PDI | Charge (mV) | LD₅₀ (μM) |
|---|---|---|---|---|
| DMPC | 277 ± 45 | 0.39 ± 0.01 | −9 ± 0.3 | 969 |
| DOTMA | 105 ± 2 | 0.43 ± 0.06 | 70 ± 4 | 78 |
| DOTMA:DOPE (1:1) | 104 ± 5 | 0.28 ± 0.04 | 28 ± 1 | ND |
| 1:DOPE (1:1) | 89 ± 1 | 0.45 ± 0.05 | −61 ± 4 | ND |
| 2:DOPE (1:1) | 85 ± 2 | 0.29 ± 0.04 | −46 ± 2 | ND |
| 3:DOPE (1:1) | 61 ± 1 | 0.38 ± 0.01 | 44 ± 2 | ND |
| 4:DOPE (1:1) | 64 ± 0.2 | 0.28 ± 0.02 | 30 ± 7 | ND |
| 9:DOPE (1:1) | 76 ± 4 | 0.57 ± 0.01 | 38 ± 2 | ND |
| 10:DOPE (1:1) | 107 ± 2 | 0.23 ± 0.01 | 44 ± 2 | ND |
| 11:DOPE (1:1) | 93 ± 5 | 0.24 ± 0.01 | 47 ± 1 | ND |
| 12:DOPE (1:1) | 98 ± 1 | 0.34 ± 0.04 | 52 ± 3 | ND |
| 3 Lipoplex (N:P 1) | 91 ± 3 | 0.45 ± 0.01 | 27 ± 6 | ND |
| 3 Lipoplex (N:P 5) | 66 ± 1 | 0.25 ± 0.01 | 21 ± 1 | ND |
| 4 Lipoplex (N:P 1) | 225 ± 7 | 0.39 ± 0.02 | 35 ± 6 | ND |
| 4 Lipoplex (N:P 5) | 194 ± 5 | 0.22 ± 0.00 | 35 ± 3 | ND |
| 9 Lipoplex (N:P 1) | 95 ± 4 | 0.46 ± 0.01 | 19 ± 1 | ND |
| 9 Lipoplex (N:P 5) | 65 ± 2 | 0.35 ± 0.05 | 24 ± 3 | ND |
| 10 Lipoplex (N:P 1) | 102 ± 4 | 0.55 ± 0.08 | 50 ± 8 | ND |
| 10 Lipoplex (N:P 5) | 78 ± 1 | 0.44 ± 0.01 | 56 ± 5 | ND |
| DOTMA Lipoplex (N:P 1) | 97 ± 2 | 0.23 ± 0.01 | 34 ± 4 | ND |
| DOTMA Lipoplex (N:P 5) | 90 ± 1 | 0.21 ± 0.00 | 2 ± 0 | ND |
| Peptide free liposome | 113 ± 4 | 0.40 ± 0.02 | ND | ND |
| CHEMS peptide liposome | 194 ± 4 | 0.23 ± 0.02 | ND | ND |
| Dioctadecylamino peptide | 177 ± 4 | 0.22 ± 0.01 | ND | ND |
| liposome | | | | |

ND = Not determined.

In addition to size, charge and complexation, pKa is another crucial aspect of cationic lipids that has been directly correlated to the success of lipid nanoparticles in gene delivery. 19,31,32 Particularly, ionizable lipids with a pKa ranging from 6.2 to 6.4, have been shown to achieve a high degree of efficacy when used to deliver siRNA.³ To assess the pKa of the cationic TZ lipids, liposomes made from these lipids were rehydrated in buffered solutions ranging from pH 2.5 to 12 and mixed with TNS, as described in the methods section. Interestingly, the pKa of the TZ lipids differed in both cases due to the tail length (FIG. 3B), despite having the same headgroup, with lipids 3 and 4 varying by almost two units (7.37 for lipid 3 and 5.59 for lipid 4).

The mixture of cationic TZ lipids with DOPE was then used to deliver plasmid DNA into HeLa cells using a luciferase reporter vector, comparing their efficacy with free DNA and Lipofectamine 3000.³⁰ As shown in FIG. 3C, all four lipids improved plasmid transfection compared with naked plasmid, with the shorter tailed lipids (3 and 9) demonstrating better efficacy than the lipids with C18 tails (4 and 10), which concurs with the findings of Candiani et al. who reported improved transfection with shorter length tails.¹⁵ Overall, TZ lipid transfection was only modest compared to Lipofectamine, with optimal luciferase expression reaching an average of 462 RLU per mg for lipid 9 at an N:P ratio of 5 (vs. 7937 RLU per mg for Lipofectamine), and LDH release and cell viability approximating that of Lipofectamine (FIGS. 3D and E).

To confirm these findings in a more clinically relevant context, HEK293-T cells were transfected with a plasmid encoding human alpha-1 antitrypsin (hAAT) using the same lipid mixtures, and hAAT expression was assessed by ELISA. As evidenced in FIGS. 3F-3H, the cationic TZ lipids significantly improved pDNA transfection, except for lipid 10, and exhibited a similar toxicity profile to that of Lipofectamine. The data from these experiments suggests that TZ lipids may have utility in the delivery of nucleic acids and warrants further exploration of their capabilities in vivo. The efficacy of the nanoparticles did not seem to correlate with a lower pKa of the nanoparticles, which was unexpected, based on previous reports.³ However, the influence of the lipid architecture on complexation with DNA as compared to siRNA may explain the deviation from the optimally reported pKa of 6.2-6.5.³ This finding highlights the need for studying the behavior of aromatic lipids, as suggested by Martinez-Negro, et al.³³ and more specifically the behavior of cationic TZ lipids, which may also serve a role as helper lipids in more complex gene delivery platforms.

Finally, given the synthetic versatility of the divergent route to append more complex moieties, such as peptides, the immunogenicity of the lipopeptide prepared with C18 TZ linker (intermediate D) as compared to our standard immunization protocol using cholesteryl hemisuccinate as a peptide anchor in the liposomal formulation was examined.

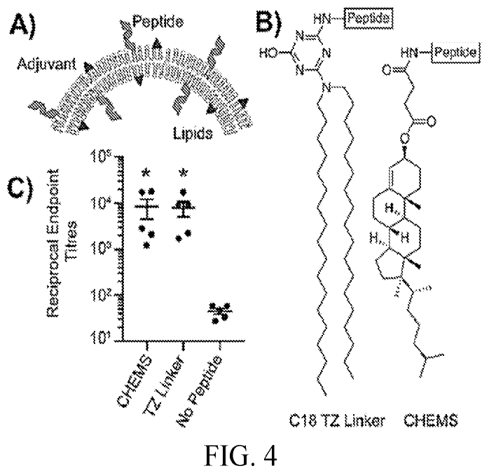

The modular design of liposomes allows for combination of antigens and adjuvants to tailor immune responses for clinically relevant immunization strategies (FIG. 4A).¹⁰ Liposomal peptide vaccines increase the bioavailability of antigens by extending their half-life and increasing concentrations in lymphatic tissues.³⁴ Our lab has previously developed a liposomal strategy to induce antibodies toward apolipoprotein A-I (ApoA-I) in mice, as a mechanism to mimic the immunity observed in humans. To achieve this, a lipid anchored 44 amino acid peptide derived from ApoA-I for liposomal immunizations was used.²² To determine whether TZ lipids could be used in this setting, formulations were prepared with the respective lipopeptides along with the adjuvant monophosphoryl lipid A (MPLA), which is a toll-like receptor (TLR)-4 agonist. Peptides are formulated in the liposomes (20 mM) at a concentration of 1 mg mL⁻¹ which equates to about 1000 peptides per liposome. The resulting liposomes range in size from 110 nm in diameter for the peptide free liposomes to between 150-200 nm for the liposomes containing peptide, which are stable for up to a month under refrigeration.

After liposome preparation, mice were immunized twice with a liposomal vaccine containing one of the lipopeptide conjugates (FIG. 4B) or a control formulation without peptide present and reciprocal endpoint titers (RET) toward the peptide immunogen were assessed seven days after the second immunization. RET from mice immunized with the TZ lipid anchor approximated that of cholesteryl hemisuccinate (CHEMS) (FIG. 4C), which has previously been shown to serve as an optimal peptide anchor for liposomal immunization.²³ These data highlight the utility of this synthetic strategy in peptide conjugation.

### Example 2: In Vivo Triazine Lipid Reagents for Plasmid DNA

A study was designed to assess the utility of TZ lipids in delivering hAAT plasmid in vivo with associated toxicity and transfection efficiency examination of these compounds in mice, using DOTAP as a comparison. Because the in vitro evaluation described above in Example 1 was based on the use of lipoplexes (LPs) it was decided to also compare these to lipid nanoparticles (LNPs), as these are reported to have improved efficacy in vivo.⁵⁹ Formulations were developed using the lipids displayed in Table 3 based on standard DOTAP formulations described previously.

TABLE 3
Structure of triazine lipids and other lipids used in plasmid formulations.

| Name | Structure |
|---|---|
| TZ3 | 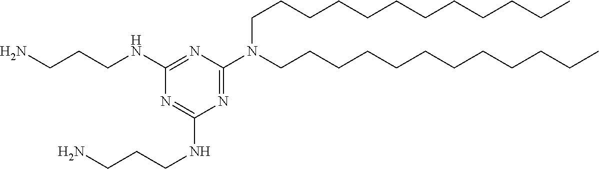 [SMILES: CCCCCCCCCCCCN(CCCCCCCCCCCC)c1nc(NCCCN)nc(NCCCN)n1] |
| TZ9 | 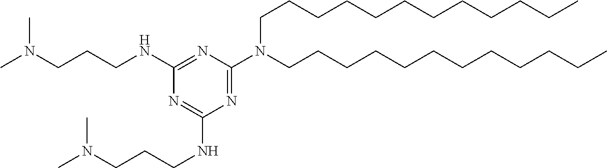 [SMILES: CCCCCCCCCCCCN(CCCCCCCCCCCC)c1nc(NCCCN(C)C)nc(NCCCN(C)C)n1] |
| DO- TAP | 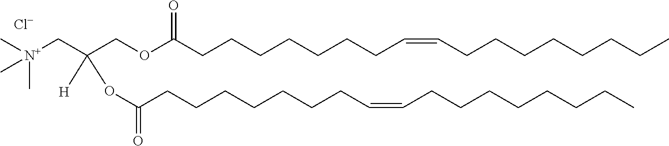 [SMILES: CCCCCCCC/C=C\CCCCCCCC(=O)OCC(C[N+](C)(C)C)OC(=O)CCCCCCC/C=C\CCCCCCCC ; [Cl-]] |
| DSPC | 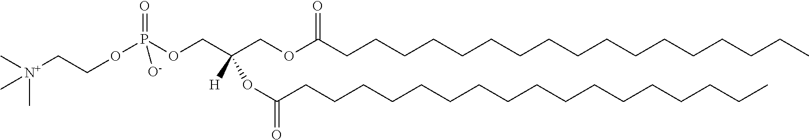 [SMILES: CCCCCCCCCCCCCCCCCC(=O)OC[C@@H](COP(=O)([O-])OCC[N+](C)(C)C)OC(=O)CCCCCCCCCCCCCCCCC] |
| DOPE | 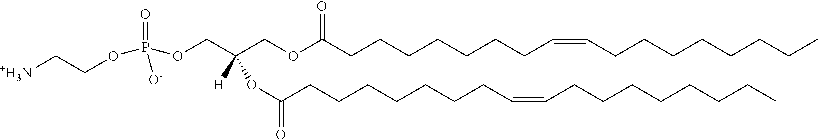 [SMILES: CCCCCCCC/C=C\CCCCCCCC(=O)OC[C@@H](COP(=O)([O-])OCC[NH3+])OC(=O)CCCCCCC/C=C\CCCCCCCC] |
| Cho- les- terol | 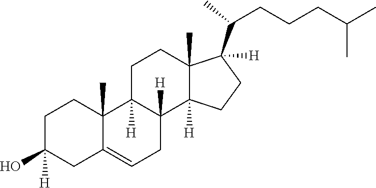 |
| DSPE- PEG (2000) | 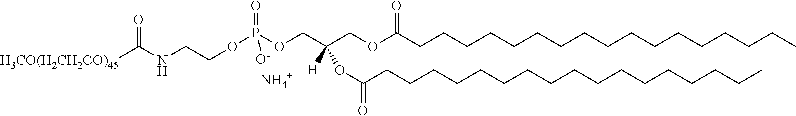 |

However, further optimization of the formulations with triazine lipids was required, leading to several novel findings. The present disclosure describes the ability of optimized TZ lipid formulations to improve in vivo plasmid transfection beyond that of standard DOTAP formulations as well as the immunologic response targeting the transgenes using each formulation. The present disclosure also reveals that lipid nanoparticles and lipoplexes including certain TZ lipid formulations induce minimal antibody profiles toward a transgene delivered therewith while serving as a platform to induce robust antibody responses when used to directly deliver a protein. Accordingly, the present disclosure thus evidences the use of TZ-based lipids as non-viral vectors for gene delivery. In the pertinent figures, LNPs are denoted as circles, while LPs are square and protein injections are triangles.

### Methods

### Mice and Cells

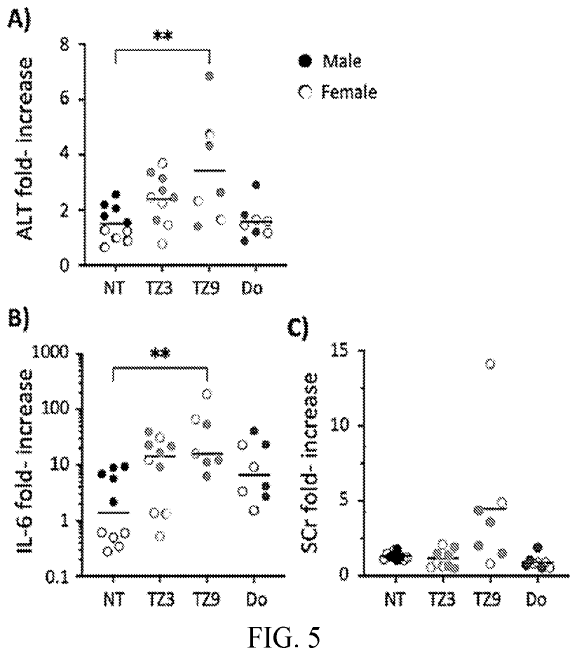

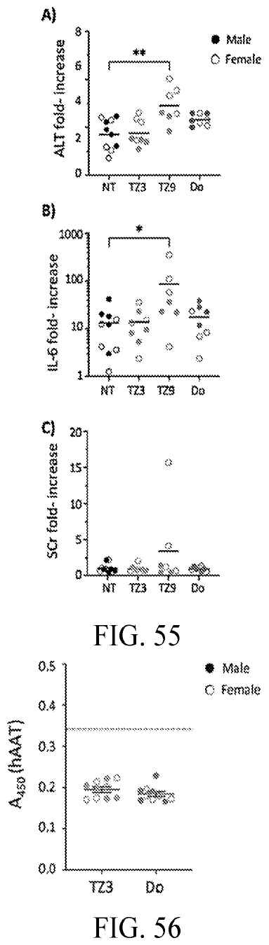

Mice were purchased from Jackson Labs at 5-6 weeks of age and used in experiments at 7-9 weeks. C57BL/6J (#000664) mice were used for toxicity experiments shown in FIGS. 5 and 55, which is incorporated herein by reference, while BALB/cJ (#000651) were used for transfections in all other figures. Equal numbers of male and female mice were used in each experiment. Mice were sedated using isoflurane gas prior to blood collection by saphenous vein puncture or intraperitoneal (i.p.) injections. Baseline serum levels of all experimental parameters were established one week prior to injections. Mice were housed in a specific pathogen free facility at the University of Kentucky, and all experimental procedures were approved by the University of Kentucky Institutional Animal Care and Use Committee #2020-3523.

HEK293T cells, kindly donated by Dr. Gregory Graf of the University of Kentucky College of Pharmacy, J774A. 1 macrophages (ATCC TIB-67) or bone marrow derived dendritic cells were used for cell experiments and maintained at 37° C. with 5% CO2.

### Development of Bone Marrow Derived Dendritic Cells (BMDCs)

Mature murine dendritic cells were obtained by culture of bone marrow monocytes as described previously⁶⁰ using recombinant murine GM-CSF (Biolegend). On day 10 of culture, lightly adherent cells were detached with gentle washing and moved to a 96-well flat bottom cell culture plate at a density of 100,000 cells per well, in triplicate, for experiments.

### Development of Lipid Nanoparticles

Two types of nanoparticles were used for experiments: liposomes and plasmid lipid nanoparticles (LNPs). In both cases, the lipids used were dissolved in chloroform, mixed at the ratio described in each figure legend, dried into a thin lipid film by rotary evaporation, and placed under house vacuum overnight before use. To form liposomes, the dried lipids were rehydrated in HEPES buffered saline (20 mM HEPES, 145 mM NaCl, pH 7) (HBS) with a pH of 4 and sonicated until translucent at 60° C. before being mixed with HBS to the final concentration and pH 7.4. Lipoplexes were formed from liposomes by mixing liposomes and DNA at a ratio of 6:1 positive to negative charges in Opti-MEM (for cells) or HBS (for mice) and incubated at room temperature for at least 12 minutes prior to administration.

To form LNPs, dried lipids were rehydrated to a concentration of 10 mM in ethanol with 10 μL of 5 M HCl per mL of ethanol and mixed with a solution of DNA at 40 ng/μL of DNA in 300 mM citric acid, pH 4. The ethanol and aqueous solutions were mixed into LNPs using the Ignite microfluidic system (Precision NanoSystems) at a ratio of 1:3 ethanol to aqueous, at a rate of 12 mL/min. The LNPs were then transferred into 3 mL Slide-A-Lyzer cassettes (ThermoFisher #PI87732) and stirred at 200 rpm in 1.5 L of a 300 mM citric acid, pH 4 solution for three hours, followed by three hours in 1.5 L of HBS buffer, pH 4 (145 mM NaCl and 20 mM HEPES), before being moved overnight to a 1.5 L solution of HBS, pH 7.4.

### Characterization of Nanoparticles

Nanoparticle size was determined using a Zetasizer Nano ZS (Malvern Panalytical) with the following settings: four measurements of fifteen, five second runs detected at a backscatter angle of 173° at room temperature. The zeta potential for the liposomes was determined in a DTS1070 folded capillary zeta cell using the following settings: four measurements of at least 50 runs modelled with the Smoluchowski equation at room temperature using the automatic settings from the instrument. The concentration of DNA in LNPs after dialysis was quantified using an AccuClear® Ultra High Sensitivity dsDNA Quantification Kit (Biotium #31027) and quantified on a BioTek Synergy H1 plate reader. Encapsulation efficiencies were determined by comparing the amount of DNA in the LNP solution vs. the DNA solution used to make them, after disrupting the LNPs with 0.5% Cl2E10 (Abcam #ab146563) and adjusting for volume differences (i.e.: excess volume added during dialysis and dilution volume during ethanol mixture).

Evaluation of In Vivo Toxicity and hAAT Transfection Efficiency

Mice were administered 0.1 mL of the liposomal solution i.p. 48 hours later, the mice were bled for evaluation of serum creatinine (SCr; Crystal Chem no. 80350), alanine aminotransferase (ALT; AAT Bioquest no. 13803) and interleukin-6 (IL-6; Biolegend no. 431304) according to manufacturer instructions.

To assess in vivo hAAT transfection efficiency, mice were administered 200 μL nanoparticles or PBS vehicle i.p. at the doses indicated in the figure legend. Seventy-two hours after injection the mice were bled again for assessment of ALT levels and hAAT expression. hAAT levels were determined via ELISA using serum diluted 1:1 in PBS containing 0.05% casein (PBS-C; 124250; Beantown Chemical), as described previously.³⁸

### Flow Cytometry

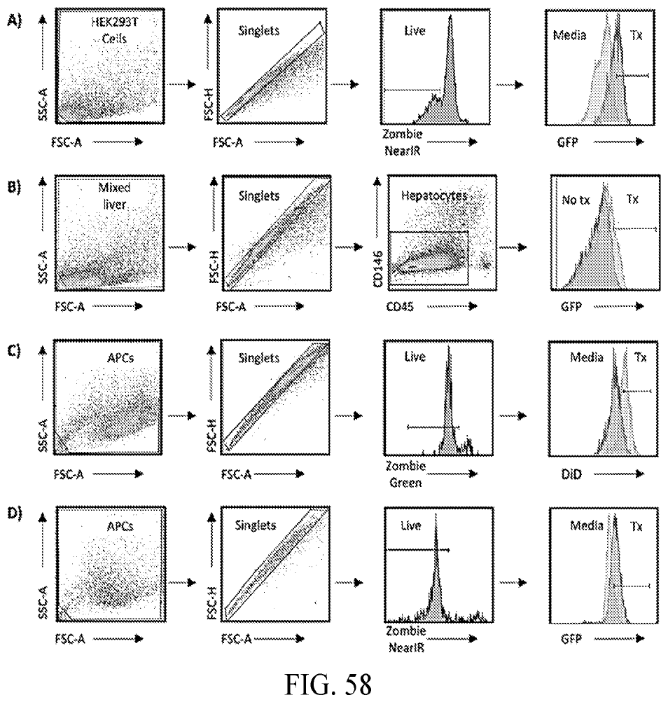

To measure transfection efficiency and subsequent GFP expression in vitro, 5×104 HEK293T or J774A.1 cells, or 1×105 mature dendritic cells, were plated in 96-well flat-bottom sterile cell culture plates and allowed to become confluent or adhere overnight. The next day, the cells were treated with 200 ng of pDNA encoding for GFP (Addgene product number 37825), delivered via nanoparticles, and incubated overnight with the nanoparticles. The media was changed at 24 hours, and after 72 total hours, cells were trypsinized briefly and transferred to a round bottom 96 well plate for flow cytometric analysis of viability and GFP expression. Live/dead staining was performed using Zombie viability dye (Biolegend) according to manufacturer instructions. Cells were washed and resuspended in FACS buffer (Mg²⁺/Ca²⁺-free Hanks' balanced salt solution, 2 mM EDTA, 25 mM HEPES and 1% FBS) for fluorescence measurement. The gating schemes used for all flow cytometry are shown in FIG. 58.

DiD liposome uptake was assessed 24 hours after liposome treatment after washing cells three times with PBS to remove free liposomes prior to trypsinization and staining as described above.

For in vivo evaluation of GFP transfection, mice were administered GFP plasmid (Addgene no. 37825) i.p. using LNPs or LPs at a dose of 10,000 ng of DNA or AAV8 at a dose of 2×109 genome copies per mouse (equating to approximately 200 ng of DNA) and serum was collected 3 days later to evaluate ALT levels as described above. Seven days after transfection, mice were euthanized by CO2 inhalation and perfused with 10 mL of Ca2+/Mg2+-free HBSS followed by 10 mL of HBSS containing 1 mg/mL type IV collagenase (MP Biomedicals) via the hepatic portal vein. Livers were excised, diced with a scalpel, and incubated for 30 minutes at 37° C. in RPMI containing collagenase at 1 mg/mL and 50 μg/mL DNAse (MP Biomedicals). Digested liver fragments were gently pressed through a 0.22 μm mesh filter and the cells were collected, centrifuged at 50×g for 3 minutes with the supernatant discarded, and then washed twice more with phosphate buffered saline. The remaining cell suspension (50 μL) from each liver was then moved to polycarbonate tubes and diluted 1:10 in FACS buffer containing anti-mouse CD16/32. After blocking, samples were incubated with fluorescent antibodies directed against mouse CD45 and CD146 for 30 minutes at 4° C. After 30 minutes, the cells were washed twice in FACS buffer before being resuspended for fluorescence measurement.

All flow cytometry antibodies, as well as viability dyes, were purchased from Biolegend. Fluorescence measurement was performed using an Attune NXT flow cytometer (ThermoFisher).

### Quantification of Anti-hAAT Antibody Titers and Determination of Subclass Ratios

To assess the presence of antibodies toward hAAT, 50 μL of hAAT (OriGene #RG202082) was plated at 2 μg/mL in carbonate buffer, pH 9.7, on a 96 well high binding plate (Greiner #82050-720) and incubated overnight at 4° C. The next day, the plates were washed with PBS containing 0.1% Tween-20 (PBS-T) and blocked for 1 hour with PBS-C at 37° C. After blocking, serum samples were plated at dilutions ranging from 1:100 to 1:1,000,000 and incubated at 37° C. for 2 hours. Secondary antibody (goat anti-mouse IgG HRP; Invitrogen #16066) diluted 1:2000 was applied for 30 min at 37° C., followed by a 30 min. incubation with tetramethylbenzidine (Rockland). Absorbance at 450 nm was recorded using a BioTek Synergy H1 microplate reader. Reciprocal endpoint titers were determined by plotting A450 values versus known dilutions, calculating the slope of that line, and dividing the slope by two times the average of the blank (no serum) wells.

Anti-hAAT IgG subclass ratios were assessed as described above, using a single 1:100 sample dilution and the following detection antibodies: goat anti-mouse IgG1-HRP (Abcam ab98693) at 1:10,000, IgG2a-HRP (Abcam ab98698) at 1:5000, IgG2b-HRP (Abcam ab98703) at 1:10,000 and IgG3 (Jackson 115-035-209) at 1:5000. Subclass ratios were calculated by dividing the absorbance of each subclass by that of IgG1 for each individual mouse.

### Data Analysis and Statistics

Data were organized and analysed using Graph Pad Prism v.9 for Windows. Groups were compared as described in the pertinent figure legends; *p<0.05; **p<0.01; ***p<0.001; ****p<0.0001. Only statistically significant comparisons are shown.

### Results and Discussion

Prior to conducting in vivo transfections, the toxicity of cationic TZ lipids was assessed using the two compounds that demonstrated the highest efficacy in vitro (triazine lipids 3 and 9, or TZ3 and TZ9 from Example 1 described above).³⁹ To test toxicity of TZ lipids, male and female C57BL/6J mice were administered TZ LNPs at 10 and 20 mM intraperitoneally (i.p.) in HEPES buffered saline. Seventy-two hours after administration, serum levels of alanine aminotransferase (ALT), interleukin 6 (IL-6), and creatinine (SCr) were tested and compared with baseline levels drawn one week prior (FIG. 5). Administration of LNPs formulated with 20 mM pure TZ9 lipid led to significant elevations in ALT and IL-6 and additionally, three of the ten mice in this group died. SCr levels in TZ9-treated mice were also elevated but did not reach statistical significance. ALT and IL-6 levels trended upward in mice treated with TZ3; however, neither these nor the IL-6 elevation in DOTAP-treated mice reached statistical significance. SCr levels were elevated but heterogeneous in TZ9-treated mice, while neither of the other two treatments caused SCr increases. Similarly, mice treated with 10 mM TZ9 showed statistically significant increases in ALT and IL-6, with one mouse dying in this treatment group (FIG. 55). Visual examination of internal organs at 72 hours revealed significant inflammation and swelling throughout the intestines and abdominal cavity of mice treated with TZ9 at 10 and 20 mM. The toxicity of TZ9 in vivo was unexpected, as in vitro studies indicated TZ9 to be less toxic than TZ3.³⁹ The discordant results between in vitro and in vivo studies suggest that the cause of toxicity is more complex than simple cytotoxicity, but the exact physiologic mechanism of toxicity is unclear. Thus, TZ3 was chosen to go forward for transfection experiments.

Lipid based nanoparticles are generally prepared with various lipids to afford a nanoparticle with specific properties, based on the desired application.⁶¹ Earlier literature describe lipoplexes (LPs) formed by mixing cationic liposomes with nucleic acid, while more recent literature focuses on lipid nanoparticles (LNPs) made by encapsulating nucleic acid in lipids dissolved in a water miscible organic solvent.^{36, 62} Much of the current literature in LNP delivery employs a formulation made with a mixture of 40-50% cationic lipid, ˜10% DSPC, 30-40% cholesterol and 1-10% PEGylated lipid, ^{36,63-68} with some work suggesting that a 50:10:38.5:1.5 ratio is optimal for delivery of siRNA and other RNA molecules. ^{65,69-71} Therefore, to evaluate TZ3 in the context of gene delivery, combinations were made with TZ3, DSPC, cholesterol and DSPE-PEG2000 at a 50:10:39:1 molar ratio, using DOTAP LNPs as a comparison. This formulation, was used to make nanoparticles by microfluidic mixing using an hAAT plasmid that ranged between 70-80 nm in diameter, with zeta potentials between 8-16 mV, and encapsulation efficiency above 70% (Table 4).⁶³ However, when administered to mice via tail vein injection, these formulations failed to elicit detectable hAAT protein levels (FIG. 56)

TABLE 4
Characterization of Liposomal Nanoparticles Used in Studies.

| | | | | DNA | FIG.(s) in |
|---|---|---|---|---|---|
| | | | Charge | Encapsulation | Application/ |
| Nanoparticle | Size (nm) | PDI | (mV) | (%) | Appendix B |
| TZ3 Liposomes | 67 ± 2.9 | 0.26 ± 0.06 | 34 ± 8 | N/A^{a} | 5, S1, 8, S3 |
| TZ9 Liposomes | 51 ± 0.7 | 0.26 ± 0.02 | 48 ± 0.2 | N/A^{a} | 5, S1 |
| DOTAP Liposomes | 79 ± 2.2 | 0.24 ± 0.06 | 18 ±2 | N/A^{a} | 5, S1, 8, S3 |
| TZ3-PEG2000 hAAT LNP | 79 ± 1.4 | 0.20 ± 0.01 | 8 ± 1 | 74.28 | S2 |
| DOTAP-PEG2000 hAAT LNP | 71 ± 0.6 | 0.16 ± 0.01 | 15 ± 0.1 | 79.76 | S2 |
| No PEG LNP - GFP^{b} | 290 ± 3.9 | 0.28 ± 0.04 | 18 ± 3 | 78.12 | 6 |
| PEG550 LNP - GFP | 228 ± 3.9 | 0.19 ± 0.04 | 25 ± 5 | 67.18 | 6 |
| PEG1000 LNP - GFP | 197 ± 8.2 | 0.10 ± 0.06 | 25 ± 1 | 68.84 | 6 |
| PEG2000 LNP - GFP | 133 ± 7.0 | 0.26 ± 0.05 | 20 ± 2 | 79.22 | 6 |
| TZ3 DSPC LNP - GFP | 485 ± 62 | 0.17 ± 0.08 | 15 ± 3 | 65.46 | 6 |
| TZ3 DOPE LNP - GFP | 649 ± 130 | 0.42 ± 0.09 | 15 ± 1 | 59.36 | 6 |
| DOTAP DSPC LNP - GFP | 818 ± 161 | 0.46 ± 0.07 | 9 ± 1 | 94.60 | 6 |
| DOTAP DOPE LNP - GFP | 513 ± 117 | 0.49 ± 0.15 | 20 ± 4 | 71.01 | 6 |
| TZ3 GFP LP | 158 ± 3.5 | 0.24 ± 0.03 | 33 ± 9 | N/A^{a} | 6 |
| TZ3-PEG550 hAAT LNP | 252 ± 0.9 | 0.14 ± 0.02 | 15 ± 0.4 | 63.62 | 7, S3 |
| TZ3 hAAT LP | 397 ± 9.8 | 0.39 ± 0.09 | 31 ± 2 | N/A | 7, S3 |
| DOTAP-PEG550 hAAT LNP | 216 ± 7.2 | 0.21 ± 0.04 | 14 ± 2 | 76.27 | 7, S3 |
| DOTAP hAAT LP | 1713 ± 51 | 0.49 ± 0.17 | 20 ± 8 | N/A^{a} | 7, S3 |
| DMPC Liposomes | 131 ± 1.6 | 0.25 ± 0.02 | −8 ± 2 | N/A^{a} | 8, S3 |
| TZ3-PEG550 GFP LNP | 417 ± 29 | 0.19 ± 0.02 | 18 ± 3 | 62.56 | 9^{c} |

^{a}N/A = not applicable.
^{b}LNPs formulated without PEG aggregated during dialysis and had large clumps of lipid/DNA complexes.
^{c}DiD liposomes could not be properly characterized by DLS due to fluorescence of the nanoparticles.

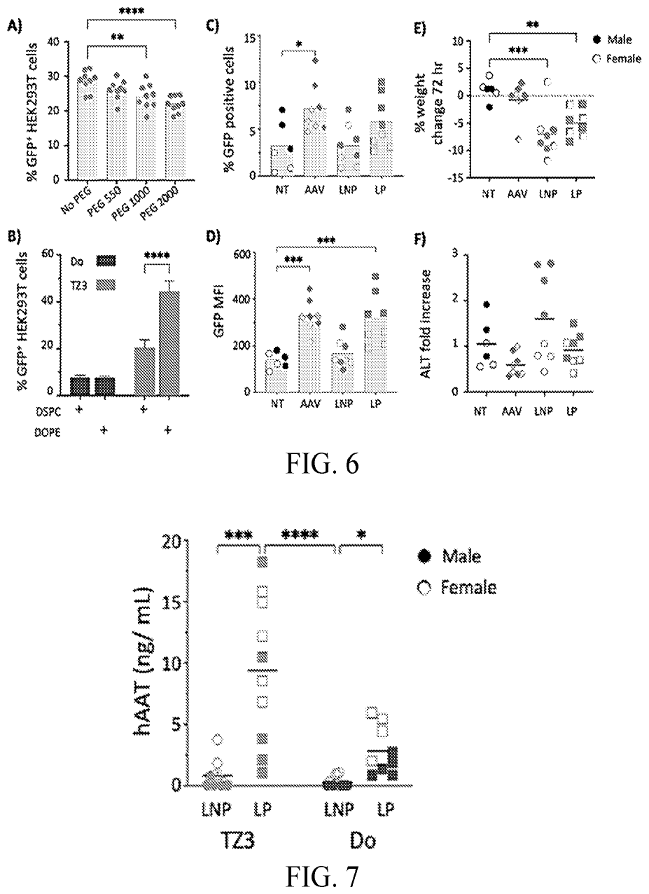

Overall, PEG content could be implicated in the poor transfection efficiency observed in FIG. 56; however, PEG has been shown to be necessary for improving circulation half-life and providing stability to nanoparticles in vivo.⁷²⁻⁷⁶ Therefore, rather than reducing the PEG concentrations, the role of PEG length on transfection efficiency was analysed using identical lipid ratios and varying lengths of PEG polymer. These formulations were then used to prepare nanoparticles encapsulating a GFP plasmid and used to transfect HEK293T cells. As shown in FIG. 6A, the length of PEG correlated with a decrease in GFP expression. The nanoparticles made without PEG or with PEG550 yielded the highest GFP expression. However, PEG-free LNPs were unstable and formed aggregates. Consequently, formulations with PEG550 were chosen for further evaluation.

Transfection may also be improved by using DOPE, rather than DSPC, as a helper lipid.^{63,70,77} To further optimize transfection, the formulations containing DSPE-PEG550 was tested using DSPC or DOPE as helper lipids, with either TZ3 or DOTAP. As shown in FIG. 6B, use of nanoparticles containing both TZ3 and DOPE significantly increased transfection efficiency, suggesting this could be the most optimal formulation for delivery.

Dynamic light scattering analysis of the nanoparticles made with PEG2000 and hAAT plasmid for the data presented in FIG. 56 exhibit similar characteristics to those described in the literature for plasmid based nanoparticles (Table 4).^{63,73,78} However, the nanoparticles made with shorter PEG chains were larger and more polydisperse, a trend that has been reported previously with the reduction of PEG2000 concentration.^{63, 73, 78} DOPE also increased size and polydispersity compared with DSPC. This change could possibly be attributed to the increased rigidity of the stearoyl tails of DSPC compared with DOPE's oleyl tails but is not known to have been previously noted. Finally, TZ3, while successful at encapsulating DNA, trended toward lower encapsulation efficiencies as compared to DOTAP, generally encapsulating 60-70% of DNA vs. DOTAP's 70-80% encapsulation. While the attributes of the nanoparticles can likely be improved by further altering multiple parameters such as cholesterol content, no additional alterations were made and further evaluation of TZ3 was pursued using PEG550 and DOPE.^{79,80}

After optimization in vitro, the PEG550 and DOPE formulation was evaluated in vivo using the same GFP plasmid. Using TZ3 LNPs, 10 μg of plasmid DNA was transfected into mice and compared with the same dose of DNA delivered via LPs made from a 1:1 ratio of TZ3 and DOPE, previously used for in vitro transfection.³⁹ Since the resulting nanoparticles were over 200 nm in diameter, they were delivered intraperitoneal (i.p.) based on the concern that intravenous (i.v.) administration could harm the animals. Additionally, previous studies have shown this route to result in similar transfection efficacy as i.v. administration.^{81,82} As shown in FIG. 6C-6D, transfection with LNPs was less efficient than that achieved using an AAV8-GFP vector, carrying the same plasmid, at a dose of 2×109 GC per mouse (˜200 ng of DNA). Although transfection with LPs was heterogeneous, mean hepatocyte GFP positivity trended upward over untreated mice. Additionally, when mice were treated with the AAV8-GFP vector or LPs, GFP MFI in hepatocytes was significantly increased over untreated mice, while LNP treatment resulted in no increase over baseline (FIG. 6D). Toxicity evaluation of these formulations showed that mice treated with LNPs and LPs lost 1-12% of their body weight at 72 hours and those treated with LNPs had slight (non-significant) elevations in ALT levels at the same timepoint (FIG. 6E-6F).

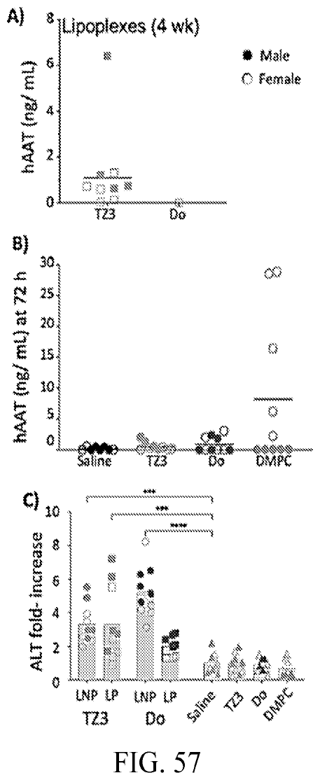

Based on these data hAAT transfection was reevaluated, using TZ3 and DOTAP LNPs or LPs. The lipid formulations were made as above and the mice received 10,000 ng of hAAT plasmid DNA. Control mice were given hAAT protein at 25 μg of protein, calculated on average observed amount of protein reported by Crepso, et al. with liposomal delivery of hAAT plasmid.^{54,82,84} Because the lipids themselves can increase immunogenicity against proteins, separate groups of mice were administered the protein in saline or with 1 mM TZ3, DOTAP, or DMPC.⁸⁵ Following transfection, the optimized LP formulation led to detectable hAAT levels in serum in some of the mice (FIG. 7), although these were well below the values reported previously for cationic lipid delivery.⁵⁷ As with GFP delivery, however, LP administration led to higher transfection efficiency with average hAAT levels of 9.5 ng/mL for TZ3 and 3 ng/mL for DOTAP LPs, which were closer to those observed in previous work by Crepso, et al. and Aliño, et al.^{57,83} hAAT levels persisted at 4 weeks after treatment, but only in TZ3 treated animals (FIG. 57). In the mice given hAAT protein with individual lipids, serum hAAT levels at 72 hours were detectable but overall lower than expected based on the reported half-life;^{86,87} however, DMPC produced an intriguing protein increase in females that was not detected in males (FIG. 57).

As with GFP transfection, toxicity of the treatments was also assessed via ALT quantification. As shown in FIG. 57, ALT levels rose 2-6 times above baseline at 72 hours. Conversely, in mice treated with protein, these signs of toxicity were not observed, suggesting that the toxicity is associated with liposomal transfection, not the lipids themselves.

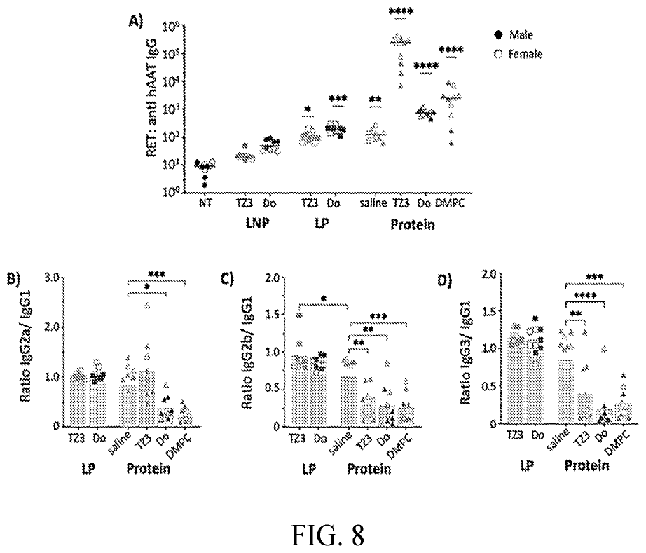

Delivery of hAAT in mice has been reported to be immunogenic; therefore, anti-hAAT reciprocal endpoint titers were also assessed two weeks after hAAT transfection (day 14).^{43,88} Delivery of the transgenic protein with LNPs produced a non-significant increase of anti-hAAT IgG titers, while mice treated with LPs made using TZ3 or DOTAP showed significantly higher anti-hAAT titers than untreated mice, approximating that of the free protein in saline (FIG. 8A). While this difference could be accounted for by the difference in protein expression between the two groups (FIG. 8), previous literature shows that protein concentrations do not necessarily correlate with titer development⁷⁰ and that protein levels may not necessarily need to reach quantifiable levels for protein to induce robust immunity.⁸⁹

Because lipids can serve as adjuvants, single lipids were also tested as above to assess the contribution of lipid to anti-hAAT immunogenicity. Administration of hAAT with DOTAP or DSPC increased titers by 10- and 100-fold, respectively. Surprisingly, TZ3 administration concurrent with hAAT protein led to an increase in titers 1000-fold higher than either of these two controls (FIG. 8A), suggesting a potential role for this compound in the setting of protein immunizations.^{90,91}

As immune biases toward a Th1- or Th2-type response following immunization can be suggestive of overall formulation immunogenicity, anti-hAAT subclass composition was also assessed via ELISA and the ratios of IgG2a, 2b and 3 to IgG1 were determined (FIG. 8B-8D) in all but LNP samples, as these did not achieve a sufficient antibody response. Transgenic hAAT delivery with both TZ3 and DOTAP led to a balanced Th1/Th2 response, as indicated by the ratios of IgG2a, 2b, and 3 over IgG1. Pure protein in saline resulted in similar responses; however, when delivered with lipids, there was a shift toward a Th2 response, indicated by ratios lower than 1.0. These data are similar to immune profile observed with Freund's incomplete adjuvant, which is known to induce a Th2 bias toward co-administered proteins.^{92,93} Interestingly, hAAT protein delivered with TZ3 resulted in a more balanced IgG2a/IgG1 ratio than DOTAP and DMPC; however, the other two subclasses did not follow suite.

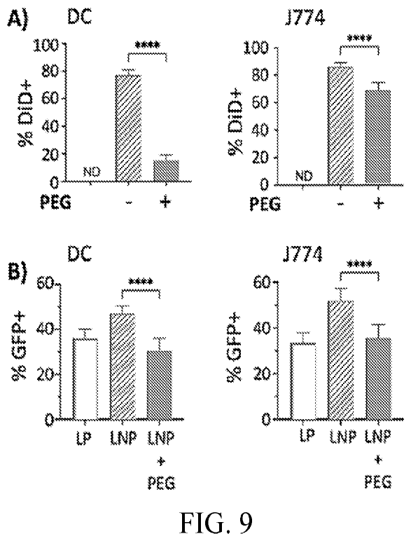

Previous work by Lu, et al. with AAV has shown that expression in antigen presenting cells (APCs) is associated with the development of antibodies against hAAT.⁸⁸ Since PEGylation of nanoparticles was originally intended to bypass the reticuloendothelial system and nanoparticle removal by APCs,^{94,95} it was hypothesized that this feature of LNPs could explain the difference in antibodies developed against LNP and LP transgenes. To test this hypothesis, 5% DiD liposomes were made with or without DSPE-PEG2000 and incubated with J774 macrophages and bone marrow derived dendritic cells (DC). After 18 hours, cells were washed to remove free liposomes and assayed by flow cytometry. The addition of PEG resulted in lower DiD fluorescence in both cell types, and most prominently in DCs, which showed more than 60% less fluorescence when PEG was included as a LNP component (FIG. 9A).

Since these LNPs were made with PEG550, which has been shown to have limited ability to inhibit APC uptake,⁹⁴ the effect of PEG on APC transfection with GFP plasmid formulations containing PEG550 was tested next. J774 macrophages and DCs were treated with either LPs, PEG free LNPs or PEG550 LNPs containing the same amount of GFP plasmid. In both cell types, transfection with PEG-free LNPs resulted in significantly higher GFP transfection than lipoplexes or LNPs containing PEG550 (FIG. 9B). In both DCs and J774 macrophages, the addition of PEG to LNPs decreased GFP positivity by more than 15%. DC expression was also slightly more efficient (˜6%) with LP treatment than PEGylated LNPs; however, this pattern was not observed with J774 macrophages. These studies collectively suggest that the addition of PEG to nanoparticles may have an advantage in reducing the immunogenicity of liposomal transgenes, but also reduce the transfection efficiency when delivering plasmid DNA.

### Conclusion

The present disclosure highlights the utility of cationic triazine lipids as a tool for in vivo research. Evaluation of in vivo toxicity of the compounds showed, surprisingly, that TZ9 confers significant toxicity and mortality via a yet unknown mechanism, which differed from the in vitro toxicity observed during transfection in Example 1.39 This toxicity not only led to elevations in liver, kidney and inflammatory markers, but also to the death of several animals. However, TZ3 showed comparable toxicity to DOTAP.

The toxicity experiments were followed by evaluation of transfection with TZ3, which demonstrated increased transfection efficacy compared with DOTAP, both in vivo and in vitro. Without wishing to be bound by any particular theory it is believed that the aromatic triazine rings of the lipids described herein are believed to improve such lipids interactions with DNA base pairs through π-π stacking, and intercalation for improved binding. 97, 98 Regardless of the functional implications of the structural characteristics of triazine lipids, TZ3 serves as a leading candidate for in vivo transfection.

While LP transfections achieved hAAT levels similar to those reported in previous lipid literature,⁵⁷ lipid-based plasmid delivery systems were not able to achieve the levels observed with viral delivery systems.^{43, 99} The hAAT plasmid used in these studies is based on a lentiviral system reported by Wilson, et al.⁹⁹ where the vector yielded protein at the microgram range, like the levels reported by Akbar, et al. with AAV.⁴³ While further optimization of the nanoparticle system, or use of other cationic lipid vectors, could improve transfections, it is also possible that plasmids designed for viral delivery require modifications to induce therapeutic protein levels using lipid nanoparticles. Plasmids offer certain advantages over other forms of nucleic acids, including longer stability and lower immunogenicity toward transgenes,^{42,44} theoretically making them better suited for long term expression of therapeutic transgenes. However, because DNA requires translocation into the nucleus and additional processing to achieve transfection, which ultimately leads to reduced levels of protein, other strategies, such as mRNA have dominated the field with the goal of improved hAAT expression using lipid-based systems.^{42, 63}

As has been demonstrated by Gael and colleagues in vaccine studies⁴⁴ and by Huysmans, et al. in protein expression kinetic experiments,¹⁰⁰ mRNA confers higher protein levels and perhaps could achieve levels of hAAT within physiological levels. In fact, a previous report of mRNA by Karadagi, et al. shows that mRNA can significantly increase hAAT levels in vitro and possibly also in vivo, although the authors do not quantify circulating levels of protein after administration into mice.¹⁰¹ Unfortunately, this would mean the need for continued mRNA delivery or self-replicating constructs, as opposed to the more stable expression achieved following delivery with viral vectors. One way to remedy this could be through optimization of plasmid delivery system, or the use of more novel systems such as CRISPR.^{102,103}

In addition to advances in mRNA delivery, much of the recent literature using LNPs for gene delivery takes advantage of ionizable lipids in formulations optimized primarily for siRNA delivery.^{36,65,74} While these compounds are greatly successful and offer many advantages to gene delivery, the present disclosure shows that formulations containing triazine lipids can provide a successful tool for plasmid delivery. Furthermore, the present disclosure shows that formulations containing DOPE and PEG550, rather than DSPC and PEG2000, can enhance the efficacy of plasmid delivery both in cells and in mice. Particularly interesting was the finding that LNPs, which contained PEG, reduced titers against the transgene compared with LPs without PEG. While the antibody response to hAAT is relatively low, these data suggest a need for further interrogation of the role of PEG in cationic lipid vaccines. Although the present disclosure shows that PEG can reduce nanoparticle uptake and transfection in antigen presenting cells (APCs), PEG is recognized by B cells in vivo,¹⁰⁴ which could help increase uptake and expression of antigens in B cells that recognize the polymer as an epitope and counter the reduced uptake by phagocytes. Another confounding factor for our evaluation of these findings is that, as reported by Hassett, et al., differences in nanoparticle size can affect titers generated by mRNA vaccines.¹⁰⁵

In addition to the modest increase in immunogenicity toward the transgene when delivered as a lipoplex, TZ3 also resulted in robust antibody induction (RET>10\hat{ }5 (or >100,000)) when used to deliver the hAAT protein. Cationic lipids are known to possess immunomodulatory properties^{85,106} and serve as adjuvants, 100 but the significant induction with TZ3 was an unexpected finding. This is particularly notable given that TZ3 induced an antibody response two orders of magnitude greater than DOTAP.

### Example 3: Triazine-Based Lipids as Vaccine Carriers, Adjuvants, Th1/Th2 Balancers, and NF_{K}B Modulators

Standard approaches to generate lipid-based carriers for vaccine design focus on inducing robust immune responses in laboratory animals as measured by increased expression of pro-inflammatory cytokines and antigen-specific antibody responses without a clear understanding of mechanism of action.¹⁰⁸ Efforts to elucidate the mechanism of action of cationic lipids suggest that 1,2-dioleoyl-3-trimethylammonium propane (DOTAP) enhances dendritic cell activation as determined by CD80 expression, with reduced TNFα expression when stimulated with LPS.¹⁰⁹ Structure activity relationships indicate that cationic lipids with short chain (e.g. C12, C14) or unsaturation (e.g. C18:1) result in enhanced dendritic cell activity.¹⁰⁹ These data suggest that cationic lipids may be inhibiting the canonical NF_{K}B pathway, however no studies have demonstrated NF_{K}B transcriptional activity in the context of cationic lipid treatment. Evidence of an interferon (IFN)-dependent mechanism has also been suggested along with a correlation with TLR9-induced canonical NF_{K}B activation.¹¹⁰ However, these studies utilize an HEK-reporter cell line with phosphatase expression driven by both canonical NF_{K}B and activator protein 1 (AP-1), which does not preclude the activation and signaling through alternative receptors.¹¹⁰ Importantly, the non-canonical NF_{K}B pathway is known to regulate IFN-dependent antiviral immunity, but this pathway is not considered in studies with DOTAP.^{111,112} Nevertheless, these data indicate that cationic lipids, specifically DOTAP, modulate NF_{K}B through an unknown mechanism which is complicated by the potential for TLR signaling alone or in combination with TLR agonists commonly used in vaccine development.

Traditional approaches to vaccine development have focused on activation of the canonical NF_{K}B pathway, which is responsible for broad pro-inflammatory responses leading to variability among patients in both immunity and reactogenicity.¹¹³⁻¹¹⁵ However, the prior data using cationic lipids suggests that the target of action may be the non-canonical NF_{K}B pathway, which drives germinal center formation, productive interactions between B and T cells, antibody isotype switching, and affinity maturation,¹¹⁶ marked by costimulatory molecule expression (e.g. CD80),¹⁰⁹ and antigen presentation (e.g. MHCII).¹¹⁷ Support for this alternative explanation is that cationic substrates are known to modulate the cellular Inhibitor of Apoptosis-1 and 2 (cIAP1 and cIAP2), which are ubiquitin ligases that activate the canonical and inhibits the non-canonical NF_{K}B pathways under basal conditions.¹¹⁸⁻¹²² Upon depletion or inhibition of cIAP 1/2 from the cytosol, the canonical pathway is halted and the non-canonical NF_{K}B pathway proceeds resulting in increased antigen presentation, costimulatory molecule expression and reduced reactogenic cytokines (e.g. TNFα). Therefore, selective inhibition of the cIAP axis will activate the non-canonical NF_{K}B pathway and enhance vaccine induced immunity without inducing unwanted reactogenicity.¹²³ Uncoupling the NF_{K}B pathways in this manner have recently been reported,¹²⁴⁻¹²⁷ but such efforts have not been examined in the context of cationic lipid based vaccine platforms.

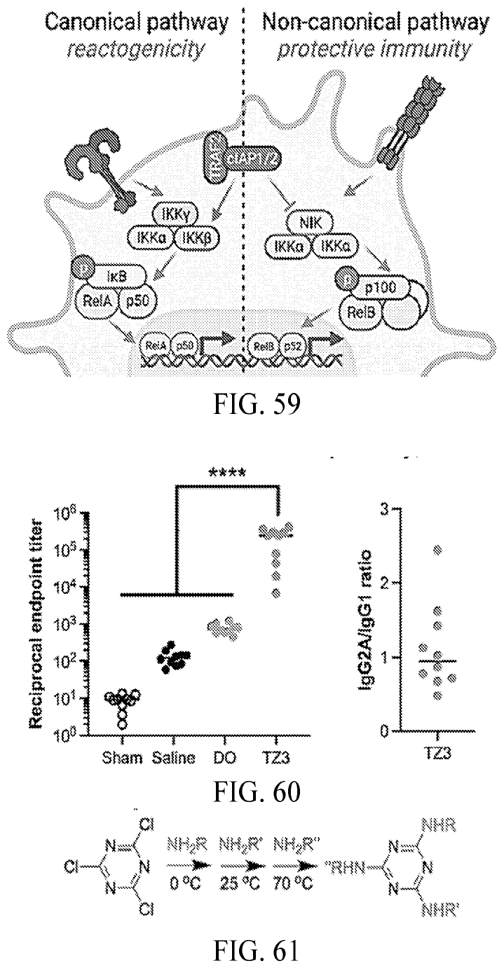

The NF_{K}B pathway is composed of a family of transcription factors including p50, p52, p65 (RelA), RelB, and c-Rel that mediate immune activation in the cell.¹¹³⁻¹¹⁵ In addition, to the five NF_{K}B inducible family members, NF_{K}B signaling is also controlled by a series of kinases, ubiquitinases, and inhibitory molecules that regulate nuclear translocation and transcription of NFKB associated genes.¹¹³ NF_{K}B signaling occurs through two pathways, the canonical pathway composed of p50, RelA, and c-Rel, and the tightly regulated and inducible non-canonical pathway composed of p52 and RelB (FIG. 59).¹¹⁶ Canonical stimulation through pattern recognition receptors result in rapid activation and transcription of pro-inflammatory gene signatures (e.g. IL-6, TNFα).¹¹³ Conversely, the non-canonical pathway is tightly regulated and activated through the TNF receptor superfamily members (e.g. CD40) resulting in germinal center formation, B/T cell interactions, and antibody affinity.¹¹⁶ Stimulation of the non-canonical pathway is critical for protective vaccine-induced immunity.^{112, 142} Targeting NF_{K}B using small molecule modulators has recently been investigated as a method of enhancing antigen specific immune responses while reducing pyrogenic cytokine production.¹¹⁴⁻¹²⁷ Although the target of these studies have not been identified, the lynchpin that regulates the canonical and non-canonical pathways are the ubiquitin ligases, cIAP1/2, which activate the canonical pathway and inhibit the non-canonical pathway under basal conditions.^{119-121, 143} Therefore, vaccine platforms that inhibit cIAP activity will inhibit the canonical NF_{K}B pathway and promote activation of the non-canonical pathway to enhance the quality of vaccine-induced immunity.

Cationic lipids have been shown to limit pyrogenic cytokine expression when stimulated with LPS or TNFα, while enhancing in vivo immune responses toward antigens.¹⁰⁹ Similarly, TZ3 demonstrates similar in vivo activity in the absence of additional adjuvants (FIG. 60).¹⁴⁴ These data, along with evidence using DOTAP and other lipids, suggest that cationic lipids inhibit the canonical NF_{K}B pathway, while stimulating the non-canonical NF_{K}B pathway.^{109,110} The chemistry used to synthesize triazine-based lipids provides an efficient synthetic strategy to achieve compositional diversity to generate structure activity relationships and elucidate its mechanism of action.¹⁴⁵⁻¹⁴⁷ Although the biological activity of TZ3 was unexpected, these data replicate previous reports of the immunomodulatory properties of cationic lipids,¹⁰⁹ and align with the hypothesis that cationic lipids are activating the non-canonical NF_{K}B pathway through inhibition of cIAP or an associated protein (i.e. TWEAK, Fn14).¹¹⁹⁻¹²¹ The robust preliminary data and synthetic ease to prepare these lipids enables a combinatorial synthetic approach to facilitate a targeted examination of the mechanism of action and identify candidates for preclinical development with the goal of generating a versatile platform for robust immune responses using triazine based lipids.

In view of the foregoing, it is thus believed that triazine-based lipids inhibit the canonical NF_{K}B pathway associated with the pro-inflammatory reactogenic response, while stimulating the non-canonical NF_{K}B pathway to improve vaccine induced immunity.

### Solid-Phase Synthesis of Triazine Lipids

Efficient solution based synthetic strategies are based on the thermally controlled chemoselective reactivity of cyanuric chloride.^{145, 147, 148} Solution phase reactions rely on protecting groups to prevent side reactions and cross-linking of reactive moieties, which increases the time, materials, and waste generated to prepare the molecules.¹⁴⁵ By implementing a solid phase synthetic approach, protecting groups are not needed and several lipids can be synthesized using a combinatorial diversification approach.¹⁴⁸ The selection of head group and tail moieties is based on the biological activity observed with DOTAP and its derivatives in which short chain lipids (C12, C14) and unsaturated lipids (C18:1) increased CD80 expression and reduced TNFα expression on dendritic cells in vitro.¹⁴⁹ Utilizing the chemoselective reactivity of cyanuric chloride, the functional moieties responsible for NF_{K}B modulation in vitro can also be examined.

### Cyanuric Chloride as the Basis for Compositional Lipid Diversity.

Cyanuric chloride (trichlorotriazine) is a D3h-symmetric molecule with seemingly equivalent reactivity at each electrophilic carbon. However, nucleophilic aromatic substitution (SNAr) at any of the carbon centers causes electron density redistribution toward the other two carbons, thereby reducing the reactivity of subsequent reactive sites.¹⁴⁹ Increasing the temperature overcomes the reduced reactivity of each carbon moiety to achieve compositional diversity around cyanuric chloride as a linker (FIG. 61).¹⁵⁰ The use of commercially available headgroups and tails offers an efficient and inexpensive strategy to generate lipids of diverse composition for continued investigation.¹⁴⁵

Solid-Phase Synthesis of TZ3. Ten of the 12 lipids (TZ1-TZ12) previously synthesized are able to form stable liposomes with a range of transition temperatures (25-65° C.) for lipids containing C18 tails.¹⁴⁵ TZ3 was, in particular, identified to exhibit robust vaccine induced antibody responses and inhibit canonical NF_{K}B signaling, and, as such, served as ideal lipid to synthesize via solid phase synthesis to assess the extent to which a combinatorial lipid library can be readily created via solid phase synthesis using commercially available headgroups and tails.

Method of Synthesis. Solid-phase synthesis of TZ3 was carried out using the strategy shown in FIG. 64. Under this strategy, a resin, which, in this case, was a 2-chlorotrtityl chloride resin) is reacted with a diamine headgroup (or first amine headgroup) selected from a library in excess (in this case, for 1 h at 0° C.) to generate an amine terminated resin for nucleophilic aromatic substitution with cyanuric chloride (in this case, by reacting for 6 h at 25° C.) to yield a corresponding dichlorotriazine. After each step, the resin is thoroughly washed to remove unreacted reagents. A lipid tail selected from a lipid tail library is then added by reacting the dichlorotriazine with the selected lipid tail (in this case, for 6 h at 25° C.), thus forming a monochlorotriazine with a lipid tail. An additional amine headgroup (or second amine headgroup) is then added to the product yielded from such reaction (i.e., the monochlorotriazine) by reacting the yielded product with another amine selected from a library (in this case, for 12 h at 25-80° C.). Finally, the lipid is cleaved from the resin using mild acid conditions and evaporating the solvent (in this case, 1-5% trifluoroacetic acid in dichloromethane for 3 h at 25° C.) to produce the final lipid product. Accordingly, to synthesize TZ3 utilizing the above-described method of synthesis: the first (diamine) headgroup selected to facilitate the generation of an amine terminated resin was

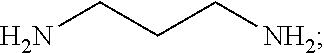

the lipid tail selected was

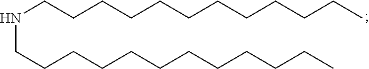 [SMILES: CCCCCCCCCCCCNCCCCCCCCCCCC]

and the second amine headgroup selected following lipid tail addition was

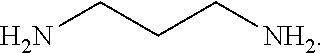

Following the above synthesis strategy, a high yield of TZ3 was synthesized without the need for column chromatography or protecting groups.

Library of Triazine-Based Lipids. A library including a variety of additional triazine-based lipids can be created by using alternative first amine headgroups, lipid tails, and/or second amine headgroups from that specified above regarding the synthesis of TZ3. For instance, in some implementations: the first (diamine) headgroup may be selected from an amine headgroup library including

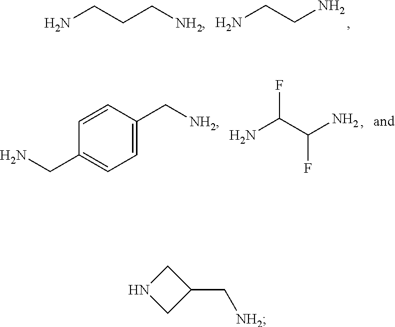

the lipid tail group may be selected from a tail group library including

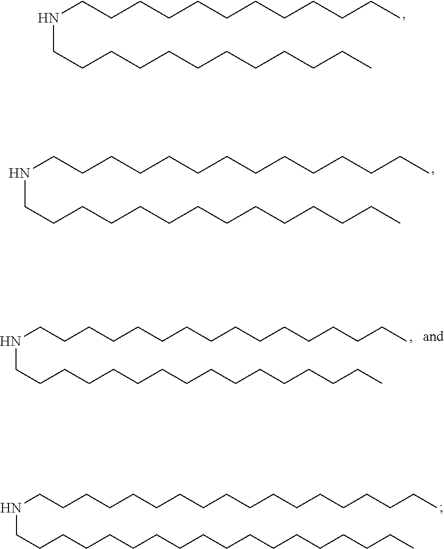 [SMILES: CCCCCCCCCCCCNCCCCCCCCCCCC ; CCCCCCCCCCCCCCNCCCCCCCCCCCCCC ; CCCCCCCCCCCCCCCCNCCCCCCCCCCCCCCCC ; CCCCCCCCCCCCCCCCCCNCCCCCCCCCCCCCCCCCC]

and the second amine headgroup may be selected from an amine headgroup library including

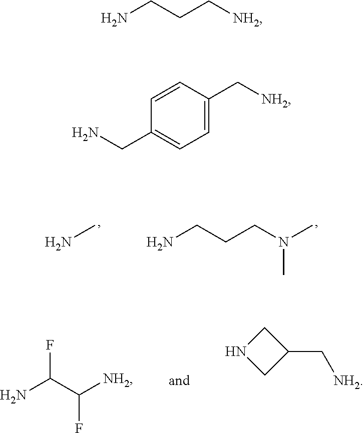

Using the foregoing libraries of headgroups and lipid tails, a total of 140 different lipids can be synthesized.

### Triazine-Based Lipids Exhibit Dose Dependent In Vitro Toxicity

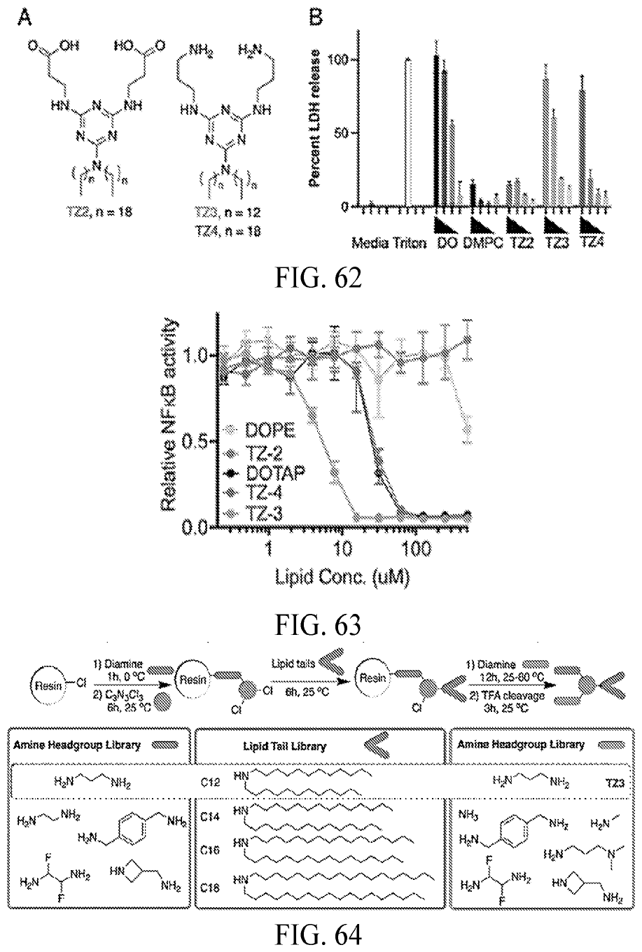

The toxicity of selected triazine lipids were assessed by lactate dehydrogenase activity as a marker of early apoptosis (FIG. 62). Murine bone marrow derived macrophages (BMDMs) were incubated with triazine lipids for 24 hours and exhibit comparable toxicity to commercially available lipids. DOTMA (LD₅₀=78 μM) and DMPC (LD₅₀=969 μM) exhibit toxicity within the previously reported range, while anionic lipids (TZ2, LD₅₀=894 μM) are relatively non-toxic and cationic lipids (TZ3, LD₅₀=133 μM; TZ4, LD₅₀=180 μM) exhibit toxicity two-to three-fold less toxic than DOTMA.

### Cationic Triazine Lipids Inhibit Canonical NF_{K}B Signaling in Human THP-1 Monocytes

THP-1-Blue NF_{K}B monocytes are an ideal tool to investigate the NF_{K}B immunomodulatory potential of compounds.¹⁵¹ These cells express secreted embryonic alkaline phosphatase (SEAP) using the NF_{K}B transcriptional response element and c-Rel binding site, which can be assessed by phosphatase activity in the media after NF_{K}B stimulation.¹⁵¹ As shown in FIG. 63, TZ3, which exhibits robust antibody responses in vivo, also exhibits canonical NF_{K}B inhibition in vitro (IC50=4 μM). TZ4 also exhibited canonical NF_{K}B inhibition in vitro, but at a higher lipid concentration (IC50=29 μM; DOTAP, IC50=28 μM). Neutral (DOPE) and anionic lipid TZ2 exhibited no activity.

### Mechanism of Triazine-Based Lipid Modulation of Canonical and Non-Canonical NF_{K}B Pathways

Cationic lipids exhibit immunomodulatory activity in vitro and in vivo.^{161, 162} Although the non-canonical pathway and cIAP have not been implicated in the modulation of cationic-lipid vaccine induced immunity, the prior data with DOTAP support the activation of the non-canonical pathway.¹⁰⁹ The signaling cascade that regulates both the canonical and non-canonical NF_{K}B pathways involves cIAP, a ubiquitin ligase, as the major regulator of each cascade.¹¹⁹-^{121, 143} During basal conditions or when a PAMP is present, cIAP1 and cIAP2 ubiquitinate scaffolding proteins in the canonical pathway resulting in their degradation, which facilitates IKKβ and IKBα phosphorylation and subsequent nuclear translocation and of the p65/p50 heterodimer for pro-inflammatory gene transcription.^{118, 143} Concomitantly, cIAP ubiquitinates the NF_{K}B-Inducing Kinase (NIK), which inhibits the non-canonical NF_{K}B pathway.¹¹⁶ Upon stimulation through TWEAK/Fn14 signaling, the cIAP binding partners, TRAF2/3, are sequestered at the cell membrane and degraded, which inhibits cIAP activity and prevents ubiqutination of both NF_{K}B pathways resulting in activation of the non-canonical pathway and inhibition of the canonical pathway.^{15, 56} Activation of the non-canonical pathway results in dendritic cell maturation, germinal center formation, B/T cell interactions, and protective immunity; however, inclusion of adjuvants that also stimulate the canonical pathway result in unwanted reactogenicity and variable protective immunity.¹¹³ Our current data suggest that TZ3 inhibits the canonical pathway, while stimulating the non-canonical pathway leading to robust antibody responses in vivo. We posit that this activity is modulated through regulation of cIAP, either directly or through an associated protein (e.g. TRAF2/3, TWEAK, Fn14).

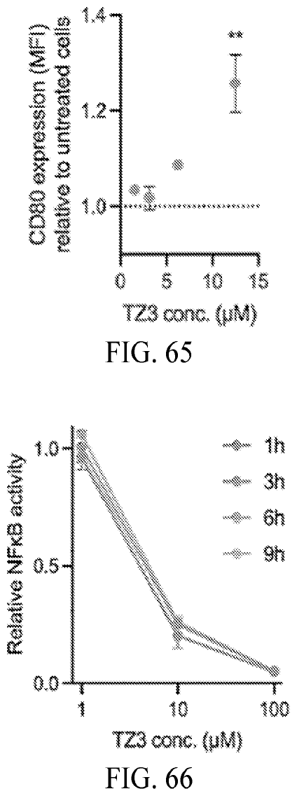

TZ3 provides robust in vivo antibody induction with a model antigen (FIG. 60),¹⁴⁴ that inhibits the canonical NF_{K}B pathway (FIG. 63), and increases expression of CD80 (FIG. 65) in TZ3 treated macrophages, which is evidence of non-canonical NF_{K}B stimulation. These data are coupled with our previous findings and expertise in evaluating the mechanism of action of small molecules that inhibit IKKβ phosphorylation, and p65/p50 nuclear translocation 164

### Cationic Triazine Lipids Promote CD80 Expression on Murine Macrophages In Vitro.

J774 murine macrophages incubated with TZ3 alone (FIG. 65) or co-treatment with TZ3 and LPS (data not shown) for 16 hours result in a dose-dependent increase in expression of the co-stimulatory molecule, CD80. Notably, cells are viable across lipid concentrations as determined with ZombieDye live/dead stain. Increased expression of CD80 after TZ3 treatment aligns with data observed for DOTAP,¹⁰⁹ and supports the hypothesis that cationic lipids activate the non-canonical NF_{K}B pathway leading to enhanced antigen presentation and co-stimulatory molecule expression.

### Cationic Triazine Lipids Exhibit Rapid Dose-Dependent Canonical NF_{K}B Inhibition.

THP-1-NF_{K}B-Blue cells treated with TZ3 result in a dose dependent inhibition of the canonical NF_{K}B pathway when treated for 16 hours (FIG. 63). Here, the duration of treatment in cells prior to stimulation with TNFα was examined from 1 to 9 hours to determine the temporal effects of cationic lipid treatment. TZ3 was compared to other cationic triazine lipids and DOTAP resulting in similar activity across all timepoints indicating a rapid onset of canonical NF_{K}B inhibition, which indicates TZ3 is most likely modulating NF_{K}B activity through inhibition of a protein target (FIG. 66).

Modulation of NF_{K}B Signaling Pathways are Monitored in Macrophages Treated with Inhibitors

We have previously evaluated the NF_{K}B inhibitory activity in vitro using azithromycin as a small molecule immune modulator (FIG. 67).⁵⁷ Notably, incubation of murine macrophages with azithromycin increases the overall expression of I_{K}β kinase (IKKB), a kinase in the signaling cascade to canonical NF-_{K}B activation. When cells were stimulated with IFNγ and LPS, AZM increased IKKβ phosphorylation despite reduced signaling resulting in inhibition of p65 translocation into the nucleus.¹⁶⁴ This work highlights the utility of these assays to unravel the role of TZ3 on modulation of the canonical and non-canonical NF_{K}B pathways

In view of the above, it is thus believed that NF_{K}B signaling can be regulated by a cationic triazine lipid, such as TZ3, through cIAP. In this regard, it is expected that the treatment of bone marrow derived dendritic cells (BMDC) with a cationic triazine lipid, such as TZ3, will result in the cationic triazine lipid inhibiting the ubiquitin ligases, cIAP1 and cIAP2, which are responsible for regulating the non-canonical NF_{K}B pathway, leading to activation of the of the non-canonical NF_{K}B pathway, while inhibiting canonical NF_{K}B activity. Provided such belief is correct, NIK accumulation in treated cells will increase leading to amplified phosphorylation of non-canonical pathway proteins (i.e., IKKα, p100), while phosphorylation of canonical NF_{K}B proteins (i.e., IKKβ, I_{K}Bα) will decrease.^{113, 115, 116} To test this expected outcome, BMDCs will be treated with TZ3 at the IC80 (10 μM) introduced to cells as a liposome for different timepoints (5, 15, 30, 45, 60 min) with or without subsequent stimulation with TNFα. Western blot of protein will be performed using the JESS ProteinSimple Automated Western Blot Analysis System (Bio-Techne) for multiplex fluorescent detection of proteins and phosphoproteins from cell lysates. Cells will be evaluated for cIAP1 and cIAP2 along with canonical NF_{K}B proteins (IKKβ/pIKKβ, I_{K}Bα/pI_{K}Bα, p65, p50) and non-canonical NF_{K}B proteins (NIK, TRAF2/3, IKKα, RelB, p100, p52) using antibodies specific for each (Abcam, Cambridge, UK). Cells not treated with TZ3, and those not stimulated with TNFα will be used as control, as well as a DOTAP comparison to examine the impact of other cationic lipids in regulating NF_{K}B signaling. Data will be compared to cells treated with birinapant, a known pharmacologic inhibitor of cIAP, at its IC50 (50 nM).¹⁶⁵

Induction of Robust Antigen Specific Adaptive Immune Response without Adverse Systemic Inflammatory Effects

Cationic lipids are known to possess adjuvant properties in vivo, but their mechanism of action remains unknown. Stimulation of the non-canonical NF_{K}B pathway results in dendritic cell maturation, germinal center formation, B/T cell interactions, affinity maturation, and protective immunity.¹¹⁶ Therefore, one explanation for the adjuvant properties of cationic lipids is through modulation of the NF_{K}B pathways. We have recently published that the triazine lipid TZ3 induces a robust and balanced antibody response toward alpha-1-antitrypsin (AAT) after a single immunization when administered intraperitoneally to mice.¹⁴⁴ Although immunization was not the goal of the prior study, this serendipitous observation suggested that triazine lipids exhibit bioactive properties that require further investigation. The hypothesis that TZ3 stimulated NF_{K}B through PRRs (e.g. TLR, NOD) was proven incorrect as the lipids inhibit canonical NF_{K}B in vitro.

As shown in FIG. 60, we have demonstrated robust in vivo immunogenicity with minimal toxicity for TZ3 and balanced Th1/Th2 immune profiles.¹⁴⁴ The observation that TZ3 inhibits canonical NF_{K}B signaling while inducing robust vaccine induced immunity supports the hypothesis that cationic triazine-based lipids will induce robust antigen specific adaptive immune response without causing adverse systemic inflammatory effects.

It will be understood that various details of the presently disclosed subject matter can be changed without departing from the scope of the subject matter disclosed herein. Furthermore, the foregoing description is for the purpose of illustration only, and not for the purpose of limitation.

All publications, patents, and patent applications mentioned in this specification are herein incorporated by reference to the same extent as if each individual publication, patent, or patent application was specifically and individually indicated to be incorporated by reference, including the references set forth in the following references list:

### REFERENCES

1. A. G. Kohli, P. H. Kierstead, V. J. Venditto, C. L. Walsh and F. C. Szoka, J. Controlled Release, 2014, 190, 274-287.
2. T. M. Allen and P. R. Cullis, Adv. Drug Delivery Rev., 2013, 65, 36-48.
3. P. R. Cullis and M. J. Hope, Mol. Ther., 2017, 25, 1467-1475.
4. J. Li, X. Wang, T. Zhang, C. Wang, Z. Huang, X. Luo and Y. Deng, Asian J. Pharm. Sci., 2015, 10, 81-98.
5. M. R. Molla, A. B″ oser, A. Rana, K. Schwarz and P. A. Levkin, Bioconjugate Chem., 2018, 29, 992-999.
6. C. Y. Zhou, H. Wu and N. K. Devaraj, Chem. Sci., 2015, 6, 4365-4372.
7. C. A. Alabi, K. T. Love, G. Sahay, H. Yin, K. M. Luly, R. Langer and D. G. Anderson, Proc. Natl. Acad. Sci. U.S.A, 2013, 110, 12881-12886.
8. L. Miao, L. Li, Y. Huang, D. Delcassian, J. Chahal, J. Han, Y. Shi, K. Sadtler, W. Gao, J. Lin, J. C. Doloff, R. Langer and D. G. Anderson, Nat. Biotechnol., 2019, 37, 1174-1185.
9. J. B. Ulmer and A. J. Geall, Curr. Opin. Immunol., 2016, 41, 18-22.
10. C. R. Alving, Z. Beck, G. R. Matyas and M. Rao, Expert Opin. Drug Delivery, 2016, 13, 807-816.
11. D. S. Watson, V. M. Platt, L. Cao, V. J. Venditto and F. C. Szoka, Clin. Vaccine Immunol., 2011, 18, 289-297.
12. C. R. Alving, M. Rao, N. J. Steers, G. R. Matyas and A. V. Mayorov, Expert Rev. Vaccines, 2012, 11, 733-744.
13. G. Fujii, W. Ernst and J. Adler-Moore, Front. Biosci., 2008, 13, 1968-1980.
14. E. E. Simanek, H. Abdou, S. Lalwani, J. Lim, M. Mintzer, V. J. Venditto and B. Vittur, Proc. R. Soc. A, 2010, 466, 1445-1468.
15. G. Candiani, M. Frigerio, F. Viani, C. Verpelli, C. Sala, L. Chiamenti, N. Zaffaroni, M. Folini, M. Sani, W. Panzeri and M. Zanda, ChemMedChem, 2007, 2, 292-296.
16. O. M. Merkel, M. A. Mintzer, D. Librizzi, O. Samsonova, T. Dicke, B. Sproat, H. Garn, P. J. Barth, E. E. Simanek and T. Kissel, Mol. Pharm., 2010, 7, 969-983.
17. E. Ralston, L. M. Hjelmeland, R. D. Klausner, J. N. Weinstein and R Blumenthal, Biochim. Biophys. Acta, Biomembr., 1981, 649, 133-137.
18. J. N. Weinstein, R. Blumenthal and R. D. Klausner, Methods Enzymol., 1986, 128, 657-668.
19. M. Jayaraman, S. M. Ansell, B. L. Mui, Y. K. Tam, J. Chen, X. Du, D. Butler, L. Eltepu, S. Matsuda, J. K. Narayanannair, K. G. Rajeev, I. M. Hafez, A. Akinc, M. A. Maier, M. A. Tracy, P. R. Cullis, T. D. Madden, M. Manoharan and M. J. Hope, Angew. Chem., Int. Ed., 2012, 51, 8529-8533.
20. M. A. Akbar, D. Nardo, M. J. Chen, A. S. Elshikha, R. Ahamed, E. M. Elsayed, C. Bigot, L. S. Holliday and S. Song, Mol. Med., 2017, 23, 57-69.
21. I. Pineda-Torra, M. Gage, A. de Juan and O. M. Pello, Methods Mol. Biol., 2015, 1339, 101-109.
22. M. G. Pitts, D. Nardo, C. M. Isom and V. J. Venditto, ImmunoHorizons, 2020, 4, 455-463.
23. D. S. Watson and F. C. Szoka Jr, Vaccine, 2009, 27, 4672-4683.
24. K. B. Knudsen, H. Northeved, P. Kumar Ek, A. Permin, T. Gjetting, T. L. Andresen, S. Larsen, K. M. Wegener, J. Lykkesfeldt, K. Jantzen, S. Loft, P. Møller and M. Roursgaard, Nanomedicine, 2015, 11, 467-477.
25. K. Lappalainen, I. J″a″askel″ainen, K. Syrj″anen, A. Urtti and S. Syrj″anen, Pharm. Res., 1994, 11, 1127-1131.
26. H. H. Gustafson, D. Holt-Casper, D. W. Grainger and H. Ghandehari, Nano Today, 2015, 10, 487-510.
27. H. Wood, Nat. Rev. Neurol., 2018, 14, 570.
28. K. Rawat, P. Kumari and L. Saha, Eur. J. Pharmacol., 2021, 892, 173751.
29. A. Lechanteur, V. Sanna, A. Duchemin, B. Evrard, D. Mottet and G. Piel, Nanomaterials, 2018, 8, 270.
30. Z. Du, M. M. Munye, A. D. Tagalakis, M. D. I. Manunta and S. L. Hart, Sci. Rep., 2014, 4, 7107.
31. C. L. Walsh, J. Nguyen, M. R. Tiffany and F. C. Szoka, Bioconjugate Chem., 2013, 24, 36-43.
32. J. Zhang, H. Fan, D. A. Levorse and L. S. Crocker, Langmuir, 2011, 27, 1907-1914.
33. M. Martinez-Negro, A. L. Barran-Berdon, C. Aicart-Ramos, M. L. Moya, C. T. de Ilarduya, E. Aicart and E. Junquera, Colloids Surf., B, 2018, 161, 519-527.
34. R. Nisini, N. Poerio, S. Mariotti, F. De Santis and M. Fraziano, Front. Immunol., 2018, 9, 155.
35. J. Zabner, Adv Drug Deliv Rev, 1997, 27, 17-28.
36. P. R. Cullis and M. J. Hope, Mol Ther, 2017, 25, 1467-1475.
37. X. Hou, T. Zaks, R. Langer and Y. Dong, Nat Rev Mat, 2021, 6, 1078-1094.
38. A. Hirko, F. Tang and J. A. Hughes, Curr Med Chem, 2003, 10, 1185-1193.
39. D. Nardo, C. M. Akers, N. E. Cheung, C. M. Isom, J. T. Spaude, D. W. Pack and V. J. Venditto, RSC Adv, 2021, 11, 24752-24761.
40. G. Candiani, M. Frigerio, F. Viani, C. Verpelli, C. Sala, L. Chiamenti, N. Zaffaroni, M. Folini, M. Sani, W. Panzeri and M. Zanda, Chem Med Chem, 2007, 2, 292-296.
41. C. Pennetta, N. Bono, F. Ponti, M. C. Bellucci, F. Viani, G. Candiani and A. Volonterio, Bioconj Chem, 2021, 32, 690-701.
42. M. A. Liu, Vaccines, 2019, 7, 37.
43. M. A. Akbar, J. J. Cao, Y. Lu, D. Nardo, M. J. Chen, A. S. Elshikha, R. Ahamed, M. Brantly, L. S. Holliday and S. Song, Hum Gene Ther, 2016, 27, 679-686.
44. A. J. Geall, A. Verma, G. R. Otten, C. A. Shaw, A. Hekele, K. Banerjee, Y. Cu, C. W. Beard, L. A. Brito, T. Krucker, D. T. O'Hagan, M. Singh, P. W. Mason, N. M. Valiante, P. R. Dormitzer, S. W. Barnett, R. Rappuoli, J. B. Ulmer and C. W. Mandl, Proc Nat Acad Sci, 2012, 109, 14604-14609.
45. M. D. Buschmann, M. J. Carrasco, S. Alishetty, M. Paige, M. G. Alameh and D. Weissman, Vaccines, 2021, 9, 65.
46. E. Kenjo, H. Hozumi, Y. Makita, K. A. Iwabuchi, N. Fujimoto, S. Matsumoto, M. Kimura, Y. Amano, M. Ifuku, Y. Naoe, N. Inukai and A. Hotta, Nat Commun, 2021, 12, 7101.
47. S. Song, Chronic Obstr Pulm Dis, 2018, 5, 289-301.
48. M. R. Ehlers, Biol Chem, 2014, 395, 1187-1193.
49. K. A. Oppelt, J. G. Kuiper, Y. Ingrasciotta, V. Ientile, R. M. C. Herings, M. Tari, G. Trifirò and U. Haug, Front Oncol, 2021, 11.
50. D. C. P. Selinger, D. I. Carbery and D. J. Al-Asiry, Brit J Hosp Med, 2018, 79, 686-693.
51. M. C. McGregor, J. G. Krings, P. Nair and M. Castro, Am J Respir Crit Care Med, 2019, 199, 433-445.
52. V. Jawa, F. Terry, J. Gokemeijer, S. Mitra-Kaushik, B. J. Roberts, S. Tourdot and A. S. De Groot, Front Immunol, 2020, 11, 1301.
53. S. Sethu, K. Govindappa, M. Alhaidari, M. Pirmohamed, K. Park and J. Sathish, Arch Immunol Ther Exp (Warsz), 2012, 60, 331-344.
54. R. Dingman and S. V. Balu-Iyer, J Pharm Sci, 2019, 108, 1637-1654.
55. A. R. Tonelli and M. L. Brantly, Ther Adv Respir Dis, 2010, 4, 289-312.
56. T. R. Flotte, B. C. Trapnell, M. Humphries, B. Carey, R. Calcedo, F. Rouhani, M. Campbell-Thompson, A. T. Yachnis, R. A. Sandhaus, N. G. McElvaney, C. Mueller, L. M. Messina, J. M. Wilson, M. Brantly, D. R. Knop, G. J. Ye and J. D. Chulay, Hum Gene Ther, 2011, 22, 1239-1247.
57. S. F. Aliño, Biochemical Pharmacology, 1997, 54, 9-13.
58. K. L. Brigham, K. B. Lane, B. Meyrick, A. A. Stecenko, S. Strack, D. R. Cannon, M. Caudill and A. E. Canonico, Hum Gene Ther, 2000, 11, 1023-1032.
59. J. A. Kulkarni, P. R. Cullis and R. van der Meel, Nucleic Acid Ther, 2018, 28, 146-157.
60. K. Inaba, W. J. Swiggard, R. M. Steinman, N. Romani, G. Schuler and C. Brinster, Curr Prot Immunol, 2009, Chapter 3, Unit 3.7.
61. A. G. Kohli, P. H. Kierstead, V. J. Venditto, C. L. Walsh and F. C. Szoka, J Control Rel, 2014, 190, 274-287.
62. P. L. Felgner, T. R. Gadek, M. Holm, R. Roman, H. W. Chan, M. Wenz, J. P. Northrop, G. M. Ringold and M. Danielsen, Proc Natl Acad Sci USA, 1987, 84, 7413-7417.
63. J. A. Kulkarni, J. L. Myhre, S. Chen, Y. Y. C. Tam, A. Danescu, J. M. Richman and P. R. Cullis, Nanomed Nanotech Biol Med, 2017, 13, 1377-1387.
64. A. K. Leung, Y. Y. Tam and P. R. Cullis, Adv Genet, 2014, 88, 71-110.
65. M. Jayaraman, S. M. Ansell, B. L. Mui, Y. K. Tam, J. Chen, X. Du, D. Butler, L. Eltepu, S. Matsuda, J. K. Narayanannair, K. G. Rajeev, I. M. Hafez, A. Akinc, M. A. Maier, M. A. Tracy, P. R. Cullis, T. D. Madden, M. Manoharan and M. J. Hope, Angew Chem Int Ed Engl, 2012, 51, 8529-8533.
66. J. A. Kulkarni, M. M. Darjuan, J. E. Mercer, S. Chen, R. van der Meel, J. L. Thewalt, Y. Y. C. Tam and P. R. Cullis, ACS Nano, 2018, 12, 4787-4795.
67. L. Digiacomo, S. Palchetti, D. Pozzi, A. Amici, G. Caracciolo and C. Marchini, Biochem Biophys Res Commun, 2018, 503, 508-512.
68. C. B. Roces, G. Lou, N. Jain, S. Abraham, A. Thomas, G. W. Halbert and Y. Perrie, Pharmaceut, 2020, 12.
69. J. A. Kulkarni, D. Witzigmann, S. Chen, P. R. Cullis and R. van der Meel, Acc Chem Res, 2019, 52, 2435-2444.
70. A. K. Blakney, P. F. Mckay, K. Hu, K. Samnuan, N. Jain, A. Brown, A. Thomas, P. Rogers, K. Polra, H. Sallah, J. Yeow, Y. Zhu, M. M. Stevens, A. Geall and R. J. Shattock, J Control Rel, 2021, 338, 201-210.
71. R. Zhang, R. El-Mayta, T. J. Murdoch, C. C. Warzecha, M. M. Billingsley, S. J. Shepherd, N. Gong, L. Wang, J. M. Wilson, D. Lee and M. J. Mitchell, Biomater Sci, 2021, 9, 1449-1463.
72. E. Ambegia, S. Ansell, P. Cullis, J. Heyes, L. Palmer and I. MacLachlan, Biochim Biophys Acta—Biomembranes, 2005, 1669, 155-163.
73. R. C. Ryals, S. Patel, C. Acosta, M. Mckinney, M. E. Pennesi and G. Sahay, PLOS ONE, 2020, 15, e0241006.
74. B. L. Mui, Y. K. Tam, M. Jayaraman, S. M. Ansell, X. Du, Y. Y. C. Tam, P. J. Lin, S. Chen, J. K. Narayanannair, K. G. Rajeev, M. Manoharan, A. Akinc, M. A. Maier, P. Cullis, T. D. Madden and M. J. Hope, Mol Ther. Nucleic acids, 2013, 2, e139-e139.
75. H. Tanaka, R. Miyama, Y. Sakurai, S. Tamagawa, Y. Nakai, K. Tange, H. Yoshioka and H. Akita, Pharmaceut, 2021, 13, 2097.
76. V. Francia, R. M. Schiffelers, P. R. Cullis and D. Witzigmann, Bioconjug Chem, 2020, 31, 2046-2059.
77. Z. Du, M. M. Munye, A. D. Tagalakis, M. D. I. Manunta and S. L. Hart, Scientific Reports, 2014, 4, 7107.
78. T. Nakamura, M. Kawai, Y. Sato, M. Maeki, M. Tokeshi and H. Harashima, Mol Pharmaceut, 2020, 17, 944-953.
79. C. Webb, S. Khadke, S. T. Schmidt, C. B. Roces, N. Forbes, G. Berrie and Y. Perrie, Pharmaceut, 2019, 11.
80. G. Basha, T. I. Novobrantseva, N. Rosin, Y. Y. C. Tam, I. M. Hafez, M. K. Wong, T. Sugo, V. M. Ruda, J. Qin, B. Klebanov, M. Ciufolini, A. Akinc, Y. K. Tam, M. J. Hope and P. R. Cullis, Mol Ther, 2011, 19, 2186-2200.
81. N. Pardi, S. Tuyishime, H. Muramatsu, K. Kariko, B. L. Mui, Y. K. Tam, T. D. Madden, M. J. Hope and D. Weissman, J Control Rel, 2015, 217, 345-351.
82. E. Quagliarini, S. Renzi, L. Digiacomo, F. Giulimondi, B. Sartori, H. Amenitsch, V. Tassinari, L. Masuelli, R. Bei, L. Cui, J. Wang, A. Amici, C. Marchini, D. Pozzi and G. Caracciolo, Pharmaceut, 2021, 13.
83. J. Crepso, C. Blaya, A. Crespo and S. F. Aliño, Biochem Pharmacol, 1996, 51, 1309-1314.
84. J. Stolk, N. Tov, K. R. Chapman, P. Fernandez, W. MacNee, N. S. Hopkinson, E. Piitulainen, N. Seersholm, C. F. Vogelmeier, R. Bals, G. McElvaney and R. A. Stockley, Eur Respir J, 2019, 54, 1900673.
85. A. G. Allison and G. Gregoriadis, Nature, 1974, 252, 252.
86. M. Campos, F. Kueppers, J. Stocks, C. Strange, J. Chen, R. Griffin, L. Wang-Smith, M. Cruz, P. Vandeberg and M. Brantly, Eur Respir J, 2013, 42, P4151.
87. R. Vidal Pla, N. Padullés Zamora, F. Sala Piñol, R. Jardí Margaleff, F. Rodríguez Frías and J. B. Montoro Ronsano, Arch Bronconeumol, 2006, 42, 553-556.
88. Y. Lu and S. Song, Proc Nat Acad Sci, 2009, 106, 17158.
89. A. F. Ogata, C.-A. Cheng, M. Desjardins, Y. Senussi, A. C. Sherman, M. Powell, L. Novack, S. Von, X. Li, L. R. Baden and D. R. Walt, Clin Infect Dis, 2021, DOI: 10.1093/cid/ciab465.
90. S. Tandrup Schmidt, C. Foged, K. Smith Korsholm, T. Rades and D. Christensen, Pharmaceut, 2016, 8, 7.
91. D. Christensen, K. S. Korsholm, P. Andersen and E. M. Agger, Exp Rev Vaccines, 2011, 10, 513-521.
92. L. Habjanec, B. Halassy and J. Tomašić, Int Immunopharmacol, 2010, 10, 751-759.
93. R. D. Weeratna, M. J. McCluskie, Y. Xu and H. L. Davis, Vaccine, 2000, 18, 1755-1762
94. S. M. Moghimi and J. Szebeni, Prog Lipid Research, 2003, 42, 463-478.
95. M. L. Immordino, F. Dosio and L. Cattel, Int J Nanomed, 2006, 1, 297-315.
96. M. Martinez-Negro, A. L. Barran-Berdon, C. Aicart-Ramos, M. L. Moya, C. T. de Ilarduya, E. Aicart and E. Junquera, Colloid Surface B, 2018, 161, 519-527.
97. P. T. Wong and S. K. Choi, Int J Mol Sci, 2015, 16, 1772-1790.
98. J. Mohanty, N. Barooah, V. Dhamodharan, S. Harikrishna, P. I. Pradeepkumar and A. C. Bhasikuttan, J Am Chem Soc, 2013, 135, 367-376.
99. A. A. Wilson, G. J. Murphy, H. Hamakawa, L. W. Kwok, S. Srinivasan, A. H. Hovav, R. C. Mulligan, S. Amar, B. Suki and D. N. Kotton, J Clin Invest, 2010, 120, 379-389.
100. H. Huysmans, Z. Zhong, J. De Temmerman, B. L. Mui, Y. K. Tam, S. Mc Cafferty, A. Gitsels, D. Vanrompay and N. N. Sanders, Mol Ther—Nucl Acids, 2019, 17, 867-878.
101. A. Karadagi, A. G. Cavedon, H. Zemack, G. Nowak, M. E. Eybye, X. Zhu, E. Guadagnin, R. A. White, L. M. Rice, A. L. Frassetto, S. Strom, C. Jorns, P. G. V. Martini and E. Ellis, Sci Rep, 2020, 10, 7052.
102. C. A. Lino, J. C. Harper, J. P. Carney and J. A. Timlin, Drug Deliv, 2018, 25, 1234-1257.
103. T. Wei, Q. Cheng, Y.-L. Min, E. N. Olson and D. J. Siegwart, Nat Commun, 2020, 11, 3232.
104. G. Besin, J. Milton, S. Sabnis, R. Howell, C. Mihai, K. Burke, K. E. Benenato, M. Stanton, P. Smith, J. Senn and S. Hoge, ImmunoHorizons, 2019, 3, 282.
105. K. J. Hassett, J. Higgins, A. Woods, B. Levy, Y. Xia, C. J. Hsiao, E. Acosta, Ö. Almarsson, M. J. Moore and L. A. Brito, J Control Rel, 2021, 335, 237-246.
106. D. K. Hong, S. Chang, C. M. Botham, T. D. Giffon, J. Fairman and D. B. Lewis, J Virol, 2010, 84, 12691-12702.
107. Watrelot, A. A., et al., Immobilization of flavan-3-ols onto sensor chips to study their interactions with proteins and pectins by SPR. Applied Surface Science, 2016 371: p. 512-518.
108. Chatzikleanthous, D.; O'Hagan, D. T.; Adamo, R., Lipid-based nanoparticles for delivery of vaccine adjuvants and antigens: Toward multicomponent vaccines. Mol Pharm 2021, 18 (8), 2867-2888.
109. Vangasseri, D. P.; Cui, Z.; Chen, W.; Hokey, D. A.; Falo, L. D., Jr.; Huang, L., Immunostimulation of dendritic cells by cationic liposomes. Mol Membr Biol 2006, 23 (5), 385-395.
110. Gandhapudi, S. K.; Shi, H.; Ward, M. R.; Bush, J. P.; Avdiushko, M.; Sundarapandiyan, K.; et al., Recombinant protein vaccines formulated with enantio-specific cationic lipid R-DOTAP induce protective cellular and antibody-mediated immune responses in mice. Viruses 2023, 15 (2), 3524-3536.
111. Rubio, D.; Xu, R. H.; Remakus, S.; Krouse, T. E.; Truckenmiller, M. E.; Thapa, R. J.; et al., Crosstalk between the type 1 interferon and nuclear factor kappa B pathways confers resistance to a lethal virus infection. Cell Host Microbe 2013, 13 (6), 701-710.
112. Jin, J.; Hu, H.; Li, H. S.; Yu, J.; Xiao, Y.; Brittain, G. C.; et al., Noncanonical NF-kappaB pathway controls the production of type I interferons in antiviral innate immunity. Immunity 2014, 40 (3), 342-354.
113. Yu, H.; Lin, L.; Zhang, Z.; Zhang, H.; Hu, H., Targeting NF-kappaB pathway for the therapy of diseases: mechanism and clinical study. Signal Transduct Target Ther 2020, 5 (1), 209.
114. Dorrington, M. G.; Fraser, I. D. C., NF-kappaB signaling in macrophages: Dynamics, crosstalk, and signal integration. Front Immunol 2019, 10, 705.
115. Zinatizadeh, M. R.; Schock, B.; Chalbatani, G. M.; Zarandi, P. K.; Jalali, S. A.; Miri, S. R., The Nuclear Factor Kappa B (NF-kB) signaling in cancer development and immune diseases. Genes Dis 2021, 8 (3), 287-297.
116. Sun, S. C., The non-canonical NF-kappaB pathway in immunity and inflammation. Nat Rev Immunol 2017, 17 (9), 545-558.
117. Korsholm, K. S.; Agger, E. M.; Foged, C.; Christensen, D.; Dietrich, J.; Andersen, C. S.; et al. The adjuvant mechanism of cationic dimethyldioctadecylammonium liposomes. Immunology 2007, 121 (2), 216226.
118. Graber, T. E.; Holcik, M., Distinct roles for the cellular inhibitors of apoptosis proteins 1 and 2. Cell Death Dis 2011, 2 (3), e135.
119. Corti, A.; Milani, M.; Lecis, D.; Seneci, P.; de Rosa, M.; Mastrangelo, E.; Cossu, F., Structure-based design and molecular profiling of Smac-mimetics selective for cellular IAPs. FEBS J 2018, 285 (17), 32863298.
120. Enwere, E. K.; Lacasse, E. C.; Adam, N. J.; Korneluk, R. G., Role of the TWEAK-Fn14-CIAP1-NF-kappaB signaling axis in the regulation of myogenesis and muscle homeostasis. Front Immunol 2014, 5, 34.
121. Roehle, K.; Qiang, L.; Ventre, K. S.; Heid, D.; Ali, L. R.; Lenehan, P.; et al. cIAP1/2 antagonism eliminates MHC class I-negative tumors through T cell-dependent reprogramming of mononuclear phagocytes. Sci Transl Med 2021, 13 (594).
122. Wajant, H., The TWEAK-Fn14 system as a potential drug target. Br J Pharmacol 2013, 170 (4), 748-764.
123. Gilmore, T. D.; Herscovitch, M., Inhibitors of NF-kappaB signaling: 785 and counting. Oncogene 2006, 25 (51), 6887-6899.
124. Kim, J. Y.; Rosenberger, M. G.; Chen, S.; Ip, C. K.; Bahmani, A.; Chen, Q.; et al. Discovery of new states of immunomodulation for vaccine adjuvants via high throughput screening: Expanding innate responses to PRRs. ACS Cent Sci 2023, 9 (3), 427-439
125. Kimani, F. W.; Manna, S.; Moser, B.; Shen, J.; Nihesh, N.; Esser-Kahn, A. P., Improving the adjuvanticity of small molecule immune potentiators using covalently linked NF-kappaB modulators. ACS Med Chem Lett 2021, 12 (9), 1441-1448.
126. Moser, B. A.; Escalante-Buendia, Y.; Steinhardt, R. C.; Rosenberger, M. G.; Cassaidy, B. J.; Naorem, N.; et al., Small molecule NF-kappaB inhibitors as immune potentiators for enhancement of vaccine adjuvants. Front Immunol 2020, 11, 511513.
127. Moser, B. A.; Steinhardt, R. C.; Escalante-Buendia, Y.; Boltz, D. A.; Barker, K. M.; Cassaidy, B. J.; et al. Increased vaccine tolerability and protection via NF-kappaB modulation. Sci Adv 2020, 6 (37).
128. Pizzuto, M.; Bigey, P.; Lachages, A. M.; Hoffmann, C.; Ruysschaert, J. M.; Escriou, V.; Lonez, C., Cationic lipids as one-component vaccine adjuvants: A promising alternative to alum. J Control Release 2018, 287, 67-77.
129. Uemura, K.; Nicolotti, R. A.; Six, H. R.; Kinsky, S. C., Antibody formation in response to liposomal model membranes sensitized with N-substituted phosphatidylethanolamine derivatives. Biochemistry 1974, 13 (8), 1572-1578.
130. Kohli, A. G.; Kierstead, P. H.; Venditto, V. J.; Walsh, C. L.; Szoka, F. C., Designer lipids for drug delivery: From heads to tails. J Control Release 2014, 190, 274-287.
131. Del Giudice, G.; Rappuoli, R.; Didierlaurent, A. M., Correlates of adjuvanticity: A review on adjuvants in licensed vaccines. Semin Immunol 2018, 39, 14-21.
132. Black, M.; Trent, A.; Tirrell, M.; Olive, C., Advances in the design and delivery of peptide subunit vaccines with a focus on toll-like receptor agonists. Expert Rev Vaccines 2010, 9 (2), 157-73.
133. Alving, C. R.; Beck, Z.; Matyas, G. R.; Rao, M., Liposomal adjuvants for human vaccines. Expert Opin Drug Deliv 2016, 13 (6), 807-16.
134. Korsholm, K. S.; Petersen, R. V.; Agger, E. M.; Andersen, P., T-helper 1 and T-helper 2 adjuvants induce distinct differences in the magnitude, quality and kinetics of the early inflammatory response at the site of injection. Immunology 2010, 129 (1), 75-86.
135. Fan, J.; Jin, S.; Gilmartin, L.; Toth, I.; Hussein, W. M.; Stephenson, R. J., Advances in infectious disease vaccine adjuvants. Vaccines (Basel) 2022, 10 (7).
136. Kidd, P., Th1/Th2 balance: the hypothesis, its limitations, and implications for health and disease. Alternative Medicine Review 2003.
137. Wang, P., Natural and synthetic saponins as vaccine adjuvants. Vaccines (Basel) 2021, 9 (3).
138. Vasou, A.; Sultanoglu, N.; Goodbourn, S.; Randall, R. E.; Kostrikis, L. G., Targeting pattern recognition receptors (PRR) for vaccine adjuvantation: From synthetic PRR agonists to the potential of defective interfering particles of viruses. Viruses 2017, 9 (7).
139. Pulendran, B.; P, S. A.; O'Hagan, D. T., Emerging concepts in the science of vaccine adjuvants. Nat Rev Drug Discov 2021, 20 (6), 454-475.
140. Herve, C.; Laupeze, B.; Del Giudice, G.; Didierlaurent, A. M.; Tavares Da Silva, F., The how's and what's of vaccine reactogenicity. NPJ Vaccines 2019, 4, 39.
141. Al-Obaydi, S.; Hennrikus, E.; Mohammad, N.; Lehman, E. B.; Thakur, A.; Al-Shaikhly, T., Hesitancy and reactogenicity to mRNA-based COVID-19 vaccines-Early experience with vaccine rollout in a multi-site healthcare system. PLOS One 2022, 17 (8), e0272691.
142. Struzik, J.; Szulc-Dabrowska, L., Manipulation of Non-canonical NF-kappaB signaling by non-oncogenic viruses. Arch Immunol Ther Exp (Warsz) 2019, 67 (1), 41-48.
143. Mahoney, D. J.; Cheung, H. H.; Mrad, R. L.; Plenchette, S.; Simard, C.; Enwere, E.; et al. Both cIAP1 and cIAP2 regulate TNFalpha-mediated NF-kappaB activation. Proc Natl Acad Sci USA 2008, 105 (33), 11778-11783.
144. Nardo, D.; Pitts, M. G.; Kaur, R.; Venditto, V. J., In vivo assessment of triazine lipid nanoparticles as transfection agents for plasmid DNA. Biomater Sci 2022, 10 (24), 6968-6979.
145. Nardo, D.; Akers, C. M.; Cheung, N. E.; Isom, C. M.; Spaude, J. T.; Pack, D. W.; Venditto, V. J., Cyanuric chloride as the basis for compositionally diverse lipids. RSC Adv 2021, 11 (40), 24752-24761.
146. Chouai, A.; Venditto, V. J., Simanek, E. E. Synthesis of 2-[3,3′-di-(tert-butyoxycarbonyl)-aminodipropylamine]-4,6-dichloro-1,3,5-triazine as a monomer and 1,3,5-[tris-piperazine]-triazine as a core for the large scale synthesis of melamine (triazine) dendrimers. Org Synth, 2009, 86, 141-150.
147. Venditto, V. J.; Allred, K.; Allred, C. D.; Simanek, E. E., Intercepting the synthesis of triazine dendrimers with nucleophilic pharmacophores: a general strategy toward drug delivery vehicles. Chem Commun 2009, (37), 5541-5542.
148. Huang, A. Y. T.; Patra, S.; Chen, H.-T.; Kao, C.-L.; Simanek, E. E., Solid-phase synthesis of libraries of triazine dendrimers and orthogonal staining methods for tracking reactions on resin. Asian J Org Chem 2016, 5 (7), 860-864.
149. Simanek, E. E.; Abdou, H.; Lalwani, S.; Lim, J.; Mintzer, M.; Venditto, V. J.; Vittur, B., The 8 year thicket of triazine dendrimers: strategies, targets and applications. Proc Royal Soc London A: Math, Phys Eng Sci 2010, 466 (2117), 1445-1468.
150. Moreno, K. X.; Simanek, E. E., Conformational analysis of triazine dendrimers: Using NMR spectroscopy to probe the choreography of a dendrimer's dance. Macromolecules 2008, 41 (12), 4108-4114.
151. Zuliani-Alvarez, L.; Piccinini, A. M.; Midwood, K. S., Screening for novel endogenous inflammatory stimuli using the secreted embryonic alkaline phosphatase NF-kappaB reporter assay. Bio Protoc 2017, 7 (7).
152. Christensen, D.; Agger, E. M.; Andreasen, L. V.; Kirby, D.; Andersen, P.; Perrie, Y., Liposome-based cationic adjuvant formulations (CAF): past, present, and future. J Liposome Res 2009, 19 (1), 2-11.
153. Feather, L. A. J.; Nadella, V.; Kastner, E.; Perrie, Y.; Hilton, A. C.; Devitt, A., Development of a rapid in vitro pre-screen for distinguishing effective liposome-adjuvant delivery systems. Sci Rep 2022, 12 (1), 12448.
154. Perrie, Y.; Kastner, E.; Khadke, S.; Roces, C. B.; Stone, P., Manufacturing methods for liposome adjuvants. Methods Mol Biol 2017, 1494, 127-144.
155. Roces, C. B.; Lou, G.; Jain, N.; Abraham, S.; Thomas, A.; Halbert, G. W.; Perrie, Y., Manufacturing considerations for the development of lipid nanoparticles using microfluidics. Pharmaceut 2020, 12 (11), 1095.
156. Hamborg, M.; Jorgensen, L.; Bojsen, A. R.; Christensen, D.; Foged, C., Protein antigen adsorption to the DDA/TDB liposomal adjuvant: effect on protein structure, stability, and liposome physicochemical characteristics. Pharm Res 2013, 30 (1), 140-55.
157. Nagy, N. A.; de Haas, A. M.; Geijtenbeek, T. B. H.; van Ree, R.; Tas, S. W.; van Kooyk, Y.; de Jong, E. C., Therapeutic liposomal vaccines for dendritic cell activation or tolerance. Front Immunol 2021, 12, 674048.
158. Al-Darraji, A.; Donahue, R. R.; Tripathi, H.; Peng, H.; Levitan, B. M.; Chelvarajan, L.; et al. Liposomal delivery of azithromycin enhances its immunotherapeutic efficacy and reduces toxicity in myocardial infarction. Sci Rep 2020, 10 (1), 1-14.
159. Watson, D. S.; Platt, V. M.; Cao, L.; Venditto, V. J., Szoka, F. C., Antibody response to polyhistidine-tagged peptide and protein antigens attached to liposomes via lipid-linked nitrilotriacetic acid in mice. Clin Vaccine Immunol 2011, 18 (2), 289-297.
160. Steinman, R. M., Decisions about dendritic cells: Past, present, and future. Annual Rev Immunol 2012, 30 (1), 1-22.
161. Carmona-Ribeiro, A. M.; Perez-Betancourt, Y., Cationic nanostructures for vaccines design. Biomimetics (Basel) 2020, 5 (3).
162. Perez-Betancourt, Y.; Tavora, B.; Colombini, M.; Faquim-Mauro, E. L.; Carmona-Ribeiro, A. M., Simple nanoparticles from the assembly of cationic polymer and antigen as immunoadjuvants. Vaccines (Basel) 2020, 8 (1).
163. Winkles, J. A., The TWEAK-Fn14 cytokine-receptor axis: discovery, biology and therapeutic targeting. Nat Rev Drug Discov 2008, 7 (5), 411-25.
164. Haydar, D.; Cory, T. J.; Birket, S. E.; Murphy, B. S.; Pennypacker, K. R.; Sinai, A. P.; Feola, D. J., Azithromycin polarizes macrophages to an M2 phenotype via inhibition of the STAT1 and NF-KB signaling pathways. J Immunol 2019, 203 (4), 1021-1030.
165. Koch, P. D.; Rodell, C. B.; Kohler, R. H.; Pittet, M. J.; Weissleder, R., Myeloid cell-targeted nanocarriers efficiently inhibit cellular inhibitor of apoptosis for cancer immunotherapy. Cell Chem Biol 2020, 27 (1), 94104 e5.
166. Tsai, C. F.; Ogata, K.; Sugiyama, N.; Ishihama, Y., Motif-centric phosphoproteomics to target kinase-mediated signaling pathways. Cell Rep Methods 2022, 2 (1), 100138.
167. Humphrey, S. J.; Karayel, O.; James, D. E.; Mann, M., High-throughput and high-sensitivity phosphoproteomics with the EasyPhos platform. Nat Protoc 2018, 13 (9), 1897-1916.
168. Ngo, K. A.; Kishimoto, K.; Davis-Turak, J.; Pimplaskar, A.; Cheng, Z.; Spreafico, R.; et al. Dissecting the regulatory strategies of NF-kappaB RelA target genes in the inflammatory response reveals differential transactivation logics. Cell Rep 2020, 30 (8), 2758-2775 e6.
169. Zhuang, Y.; Ma, Y., Wang, C.; Hai, L., Yan, C.; Zhang, Y.; Liu, F.; Cai, L., PEGylated cationic liposomes robustly augment vaccine-induced immune responses: Role of lymphatic trafficking and biodistribution. J Control Release 2012, 159 (1), 135-142.
170. Stebegg, M.; Kumar, S. D.; Silva-Cayetano, A.; Fonseca, V. R.; Linterman, M. A.; Graca, L., Regulation of the germinal center response. Front Immunol 2018, 9, 2469.
171. Lyons, A. B., Analysing cell division in vivo and in vitro using flow cytometric measurement of CFSE dye dilution. J Immunol Methods 2000, 243 (1-2), 147-154.
172. Carnell, G. W.; Ferrara, F.; Grehan, K.; Thompson, C. P.; Temperton, N. J., Pseudotype-based neutralization assays for influenza: A systematic analysis. Front Immunol 2015, 6, 161.
173. Nardo, et al., In vivo assessment of triazine lipid nanoparticles as transfection agents for plasmid DNA, Biomater. Sci., 2022, 10, 6968-6979 (2022).
174. Gupta, A; Andresen, J L; Manan, R S; Langer, R. Adv Drug Deliv Rev, 2021, 113834.
175. Vangasseri et al., Immunostimulation of dendritic cells by cationic liposomes. Mol Membr Biol 2006, 23, 385-395.

1. A triazine lipid, with a formula of: 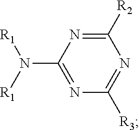
wherein R₁ is alkyl;
wherein R₂ is 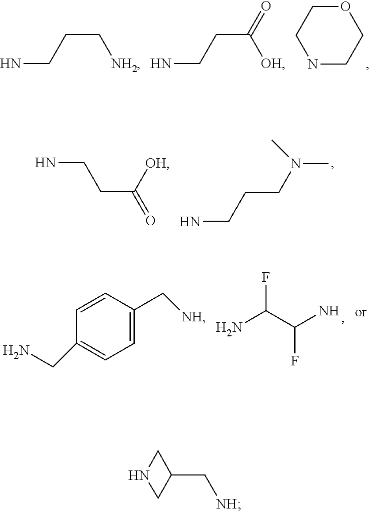
and
wherein R₃ is NH₂,

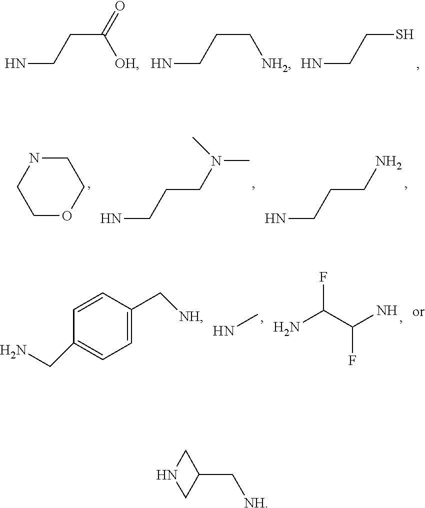

2. The triazine lipid according to claim 1, wherein R₁ comprises 18 or fewer alkyl carbons.

3. The triazine lipid according to claim 1, wherein R₁ comprises at least 12 alkyl carbons.

4. The triazine lipid according to claim 1, wherein R₁ comprises 18 alkyl carbons.

5. The triazine lipid according to claim 1, wherein R₁ comprises 12 alkyl carbons.

6. The triazine lipid according to claim 1, wherein R₂ and R₃ each comprise

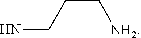

7. The triazine lipid according to claim 1, wherein the triazine lipid is

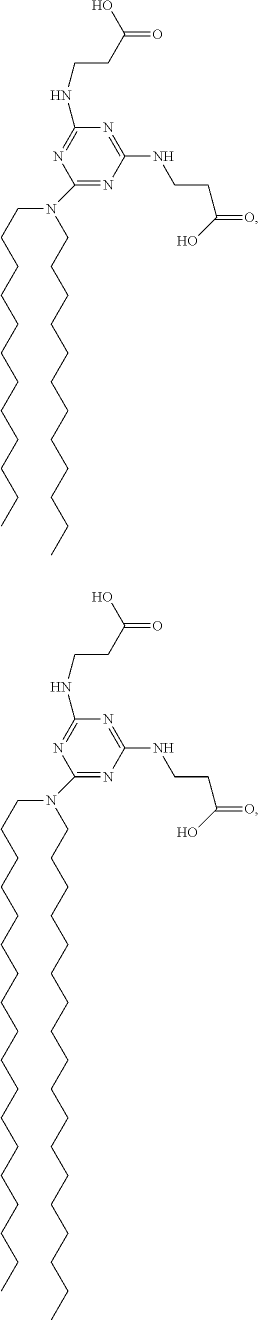 [SMILES: CCCCCCCCCCCCN(CCCCCCCCCCCC)c1nc(NCCC(=O)O)nc(NCCC(=O)O)n1 ; CCCCCCCCCCCCCCCCCCN(CCCCCCCCCCCCCCCCCC)c1nc(NCCC(=O)O)nc(NCCC(=O)O)n1]

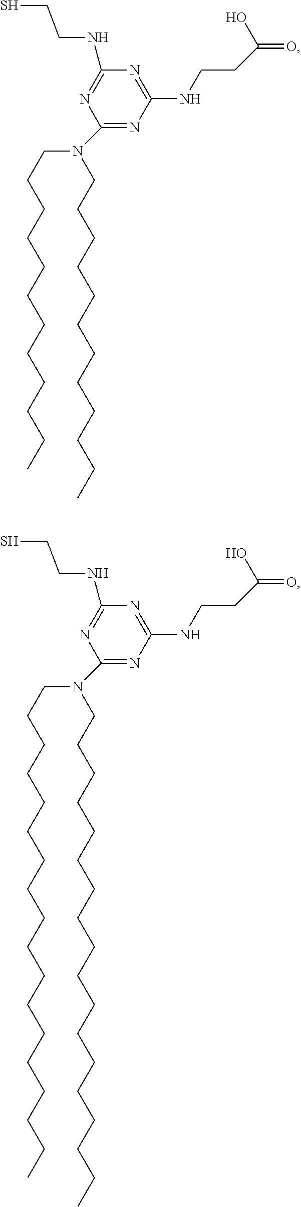 [SMILES: CCCCCCCCCCCCN(CCCCCCCCCCCC)c1nc(NCCS)nc(NCCC(=O)O)n1 ; CCCCCCCCCCCCCCCCCCN(CCCCCCCCCCCCCCCCCC)c1nc(NCCS)nc(NCCC(=O)O)n1]

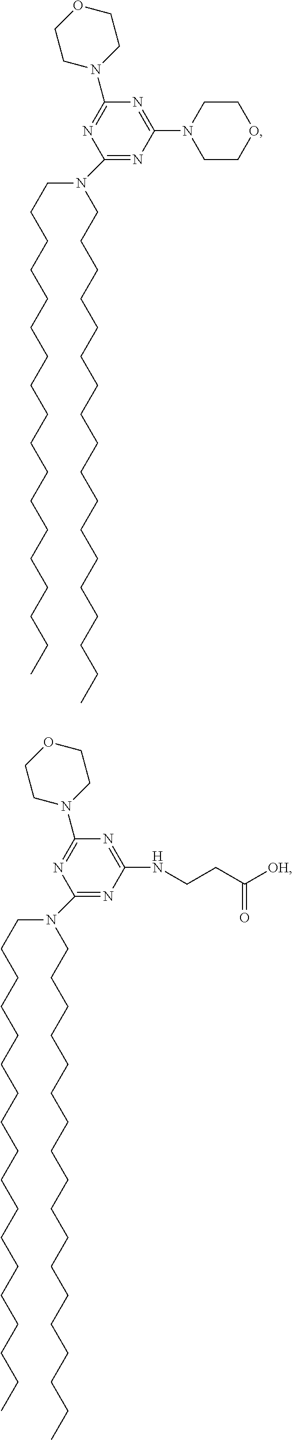 [SMILES: CCCCCCCCCCCCCCCCCCN(CCCCCCCCCCCCCCCCCC)c1nc(N2CCOCC2)nc(N2CCOCC2)n1 ; CCCCCCCCCCCCCCCCCCN(CCCCCCCCCCCCCCCCCC)c1nc(NCCC(=O)O)nc(N2CCOCC2)n1]

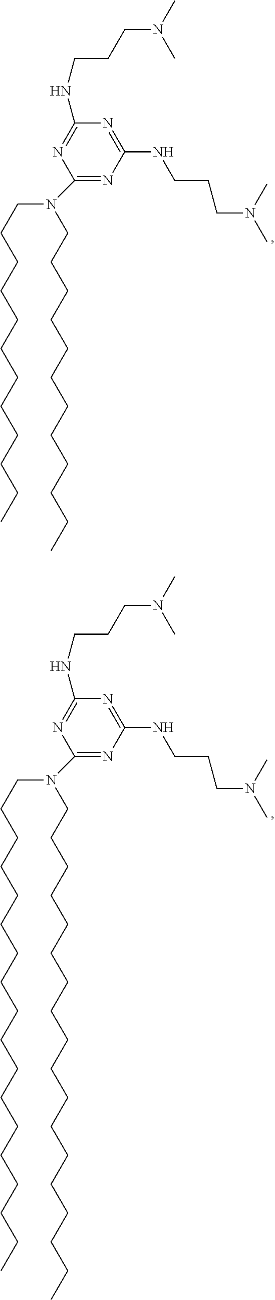 [SMILES: CCCCCCCCCCCCN(CCCCCCCCCCCC)c1nc(NCCCN(C)C)nc(NCCCN(C)C)n1 ; CCCCCCCCCCCCCCCCCCN(CCCCCCCCCCCCCCCCCC)c1nc(NCCCN(C)C)nc(NCCCN(C)C)n1]

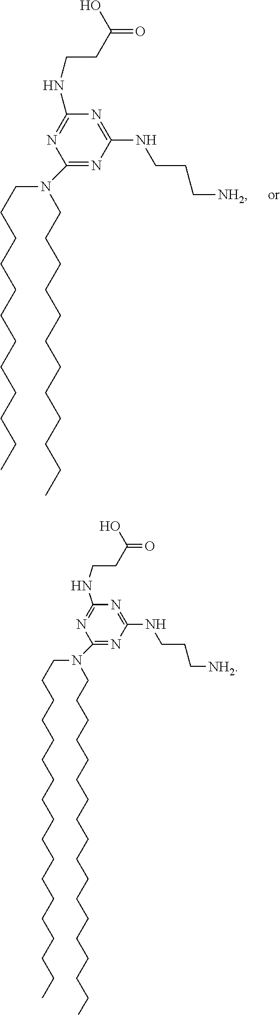 [SMILES: CCCCCCCCCCCCN(CCCCCCCCCCCC)c1nc(NCCCN)nc(NCCC(=O)O)n1 ; CCCCCCCCCCCCCCCCCCN(CCCCCCCCCCCCCCCCCC)c1nc(NCCCN)nc(NCCC(=O)O)n1]

8. The triazine lipid according to claim 1, wherein the triazine lipid is

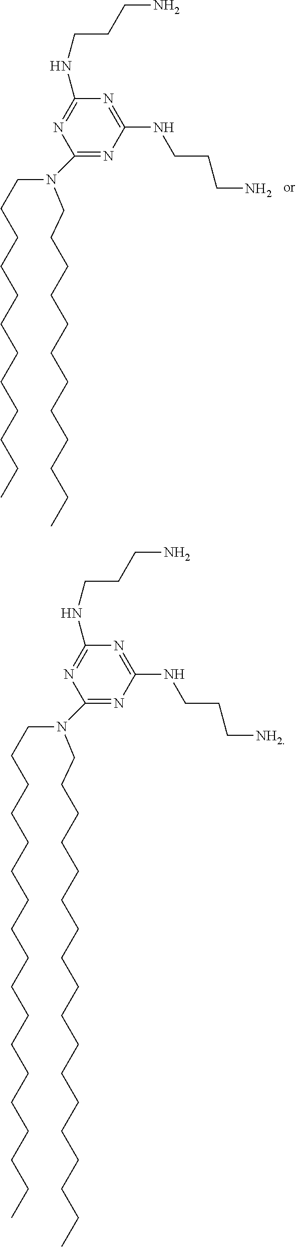 [SMILES: CCCCCCCCCCCCN(CCCCCCCCCCCC)c1nc(NCCCN)nc(NCCCN)n1 ; "or" ; CCCCCCCCCCCCCCCCCCN(CCCCCCCCCCCCCCCCCC)c1nc(NCCCN)nc(NCCCN)n1]

9. A method of synthesizing a triazine lipid, comprising:
reacting a resin with a first amine headgroup to generate an amine terminated resin;
forming a dichlorotriazine via nucleophilic aromatic substitution of the amine terminated resin with a cyanuric chloride;
reacting the dichlorotriazine with a lipid tail to form a monochlorotriazine;
reacting the monochlorotriazine with a second amine group to form the triazine lipid; and
cleaving the triazine lipid from the resin.

10. The method according to claim 9, wherein the resin is 2-chlorotrityl chloride resin.

11. The method according to claim 9, wherein the lipid tail is a saturated or unsaturated dialkylamine.

12. The method according to claim 11, wherein the lipid tail is selected from the group consisting of

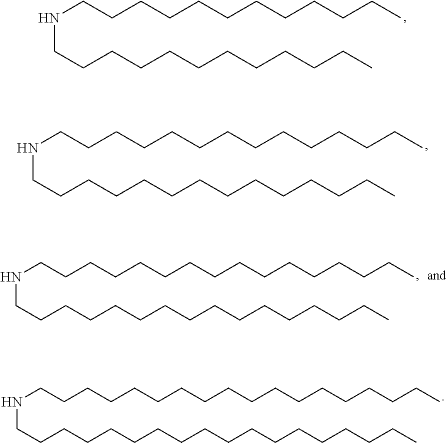 [SMILES: CCCCCCCCCCCCNCCCCCCCCCCCC ; CCCCCCCCCCCCCCNCCCCCCCCCCCCCC ; CCCCCCCCCCCCCCCCNCCCCCCCCCCCCCCCC ; CCCCCCCCCCCCCCCCCCNCCCCCCCCCCCCCCCCCC]

13. The method according to claim 9, wherein at least one of the first amine headgroup and the second amine headgroup is a diamine.

14. The method according to claim 9, wherein the first amine headgroup is selected from the group consisting of 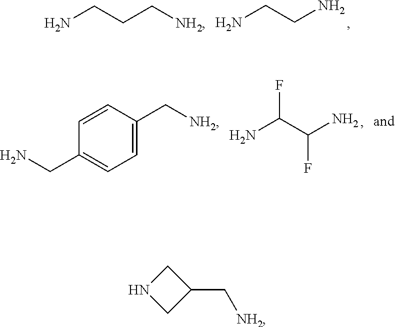
and wherein the second amine headgroup is selected from the group consisting of

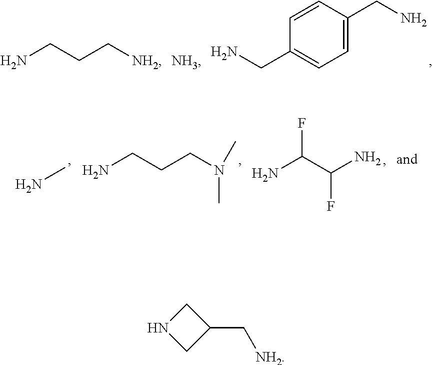

15. (canceled)

16. (canceled)

17. A method for inhibiting canonical Nuclear Factor Kappa B (NF_{K}B) transcriptional activity during an immune response to an immunostimulatory antigen within a subject, comprising:
administering a non-viral triazine lipid-based vector including a plurality of triazine lipids to the subject concurrently with the immunostimulatory antigen.

18. The method according to claim 17, wherein the immunostimulatory antigen is an immunogenic polypeptide.

19. The method according to claim 17, wherein the immunostimulatory antigen is an immunostimulatory nucleic acid.

20. The method according to claim 17, wherein each triazine lipid of the plurality of triazine lipids is cationic.

21. The method according to claim 17, wherein each triazine lipid of the plurality of triazine lipids includes a lipid tail group, a triazine linker, and a cationic head group.

22. (canceled)

23. The method according to claim 17, wherein each triazine lipid of the plurality of triazine lipids is of the formula: 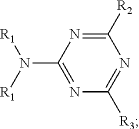
wherein R₁ is alkyl;
wherein R₂ is 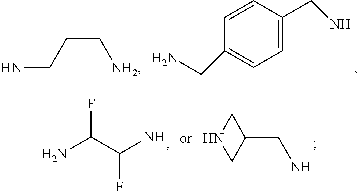
and
wherein R₃ is NH₂,

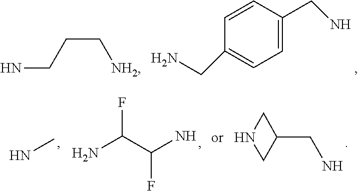

24. (canceled)

25. (canceled)

26. (canceled)

27. (canceled)

28. (canceled)

29. The method according to claim 17, wherein the non-viral triazine lipid-based vector is a liposome.

30. The method according to claim 17, wherein the non-viral triazine lipid-based vector includes one or more additional lipids selected from the group consisting of dioleoylphosphatidylethanolamine (DOPE), distearolyphosphatidycholine (DSPC), and 1, 2-distearoyl-sn-glycero-3-phsphoethanolamine-polyethylene glycol (DSPE-PEG).

31. (canceled)

32. The method according to claim 17, wherein the immunostimulatory antigen is an immunogenic polypeptide, and wherein the non-viral triazine-based vector invokes an anti-polypeptide response, and wherein the anti-polypeptide response invoked by the non-viral triazine lipid-based vector is greater than an anti-polypeptide response invoked by dioleoyl-3-trimethylammonium propone (DOTAP) or 1,2-Dimyristoyl-sn-glycero-3-phosphocholine (DMPC) when administered concurrently with the immunogenic polypeptide.

33. (canceled)

34. The method according to claim 17, wherein the non-viral triazine lipid-based vector and the immunostimulatory antigen are administered as an immunogenic composition which includes the non-viral triazine lipid-based vector and the immunostimulatory antigen.

35. The method according to claim 17, wherein the non-viral triazine lipid-based vector and the immunostimulatory antigen are administered with a pharmaceutically-acceptable carrier.

36. The method according to claim 17, wherein each triazine lipid of the plurality of triazine lipids is

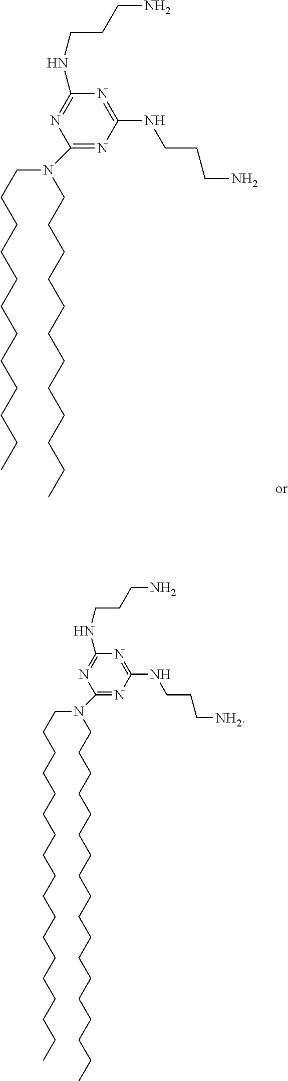 [SMILES: CCCCCCCCCCCCN(CCCCCCCCCCCC)c1nc(NCCCN)nc(NCCCN)n1 ; CCCCCCCCCCCCCCCCCCN(CCCCCCCCCCCCCCCCCC)c1nc(NCCCN)nc(NCCCN)n1]

37. A method for inhibiting canonical Nuclear Factor Kappa B (NF_{K}B) transcriptional activity during an immune response within one or more cells, comprising:
contacting the one or more cells with one or more triazine lipids of the formula of claim 1.

38. (canceled)

39. (canceled)

40. (canceled)

41. (canceled)

42. (canceled)

43. (canceled)

44. (canceled)

45. (canceled)

46. (canceled)

47. (canceled)

48. (canceled)
